SEQUENCE LISTING (1 sequence)

SEQ ID NO: 1 - protein, 1368 aa, Streptococcus pyogenes [sequence omitted]

(19) United States
(12) Patent Application Publication
(10) Pub. No.: US 2026/0234586 A1
(43) Pub. Date: Aug. 13, 2026

(54) CAS9 VARIANTS ENHANCING SPECIFICITY

(71) Applicants: THE UNIVERSITY OF NORTH TEXAS HEALTH SCIENCE CENTER, Ft. Worth, TX (US); BOARD OF REGENTS, THE UNIVERSITY OF TEXAS SYSTEM, Austin, TX (US)

(72) Inventors: Jin LIU, Ft. Worth, TX (US); Gerardo CISNEROS, Austin, TX (US); Yazdan MAGHSOUD, Austin, TX (US); Vindi M. JAYASINGHE-ARACHCHIGE, Ft. Worth, TX (US)

(73) Assignees: THE UNIVERSITY OF NORTH TEXAS HEALTH SCIENCE CENTER, Ft. Worth, TX (US); BOARD OF REGENTS, THE UNIVERSITY OF TEXAS SYSTEM, Austin, TX (US)

(21) Appl. No.: 19/155,198

(22) PCT Filed: Feb. 1, 2024
(86) PCT No.: PCT/US2024/014026

§ 371 Date: Aug. 8, 2025

Related U.S. Application Data
(60) Provisional application No. 63/444,448, filed on Feb. 9, 2023.

(51) Int. Cl. C12N 9/22; C12N 15/11; C12N 15/90; G16B 5/00

(52) U.S. Cl. CPC C12N 9/226; C12N 15/11; C12N 15/902; G16B 5/00; C12N 2310/20

(57) ABSTRACT

Certain embodiments are directed to modified or variant Cas9 proteins, and/or methods of using the same. Certain embodiments are directed to a target DNA strand comprising a C to G mismatch at a fifth position of a protospacer adjacent motif (PAM) as compared to a guide DNA strand, and/or methods of using the same.

### CROSS REFERENCE TO RELATED APPLICATIONS

This application claims priority to U.S. Application 63/444,448 filed Feb. 9, 2023, which is incorporated herein by reference.

### STATEMENT REGARDING FEDERALLY FUNDED RESEARCH

This invention was made with government support under Grant Number HL147265 awarded by the National Institutes of Health and Grant Number GM108583 awarded by the National Institutes of Health. The government has certain rights in the invention.

### SEQUENCE LISTING

The instant application contains a Sequence Listing which has been submitted electronically in ASCII format and is hereby incorporated by reference in its entirety. The file name for the Sequence Listing is “UNTHP0009 Sequence Listing.xml”, created on Jan. 23, 2024, with a size of 3,089 bytes.

### BACKGROUND OF THE INVENTION

### A. Field of the Invention

The invention relates to the field of engineered Cas9 protein structure and methods for making and using the same.

### B. Description of Related Art

The Clustered Regularly Interspaced Short Palindromic Repeats (CRISPR)/CRISPR-associated protein 9 (Cas9) system from Streptococcus pyogenes has been repurposed as a powerful and versatile genome-editing toolbox used in various living cells and organisms, demonstrating an enormous potential toward future therapeutic applications (Jiang and Doudna, Annu. Rev. Biophys., 2017; Charpentier and Doudna, Nature 495, 50-51, 2013; Mali et al. Science 339, 823-26, 2013; Cong et al. Science 339, 819-23, 2013). Guided by a chimeric single-guide RNA (sgRNA), the endonuclease Cas9 generates site-specific breaks in the double-stranded DNA (dsDNA) target (Jinek et al. Science 337, 816-21, 2012; Gasiunas et al. Proc. Natl. Acad. Sci. U.S.A. 109, E2579-86, 2012). Recognition and cleavage of dsDNA uses the presence of a protospacer adjacent motif (PAM) in the non-target DNA strand (ntDNA) and uses the base-pair complementarity of the target DNA strand (tDNA) to the RNA guide template (Jinek et al. Science 337, 816-21, 2012; Gasiunas et al. Proc. Natl. Acad. Sci. U.S.A. 109, E2579-86, 2012). Cas9 adopts an overall bi-lobed architecture, in which the sgRNA: tDNA heteroduplex resides within the central channel between the α-helical recognition (REC) and nuclease (NUC) lobes, while the displaced ntDNA threads into a side channel within thdrwine NUC lobe (Jiang et al. Science 351, 867-71, 2016; Jiang et al. Science 348, 1477-81, 2015; Nishimasu et al. Cell 156, 935-49, 2014; Anders et al. Nature 513, 569-73, 2014). The NUC lobe comprises of two metal-ion-dependent nuclease domains, dubbed as HNH and RuvC, which are responsible for cutting the tDNA (via one-metal-ion mechanism) (Yang, Q. Rev. Biophys. 44, 1-93, 2011; Yang, Nat. Struct. Mol. Biol. 15, 1228-31, 2008) and ntDNA (via two-metal-ion mechanism (Yang, Q. Rev. Biophys. 44, 1-93, 2011; Yang, Nat. Struct. Mol. Biol. 15, 1228-31, 2008; Yang et al., Mol. Cell 22, 5-13, 2006), respectively.

Capturing catalytic metal ion-containing nuclease/substrate complexes has been nontrivial for experimental means like X-ray crystallography and NMR spectroscopy, as the reaction generally occurs instantly (Yang et al., Mol. Cell 22, 5-13, 2006). It is thus not surprising that none of the Cas9 crystal structures in different binding forms solved over the past few years assumes a fully active state for either RuvC or HNH domain (Jiang et al. Science 351, 867-71, 2016; Jiang et al. Science 348, 1477-81, 2015; Nishimasu et al. Cell 156, 935-49, 2014; Anders et al. Nature 513, 569-73, 2014; Jinek et al. Science 343, 1247997, 2014).

Using molecular dynamics simulations, the catalytically competent state of RuvC domain primed for cleaving the ntDNA was reported (Zuo and Liu, Sci. Rep. 5, 2016). However, the catalytic conformation of the HNH domain for cleaving the tDNA was not captured (Zuo and Liu, Sci. Rep. 5, 2016). In contrast with the RuvC domain, the active center of HNH domain is surprisingly distant from the scissile phosphate on the tDNA in all available structures (Jiang et al. Science 351, 867-71, 2016; Jiang et al. Science 348, 1477-81, 2015; Nishimasu et al. Cell 156, 935-49, 2014; Anders et al. Nature 513, 569-73, 2014), with a separation of ~13 Å in the complete DNA duplex bound pre-catalytic state to ~46 Å in the RNA-only bound inactive state. In this respect, how to obtain a reliable catalytic state of Cas9 HNH domain has been of special focus to the experimental biologists and the computational biophysicists, as this structure can bridge an important missing link in understanding Cas9 binding, activation and cleavage mechanism, and guide structure-based Cas9 engineering with enhanced specificity (Slaymaker et al. Science 351, 84-88, 2016; Kleinstiver et al. Nature 529, 490-95, 2016). A single-molecule Förster resonance energy transfer (smFRET) study suggested that divalent metal ions are necessary for Cas9 conformational activation toward catalysis (Dagdas et al. bioRxiv, 122242, 2017). At the atomic level, however, how the metal ions aid HNH domain transition to the catalytic state remains elusive.

The knowledge of structure and dynamics of the catalytic state of HNH domain is critical for Cas9 specificity improvement. The off-target effects pose a major challenge for Cas9-mediated genome-editing applications requiring a high level of precision. Remarkably, a recent study found that CRISPR-Cas9 induced an unexpected high number of new mutations in a mouse model of gene therapy, involving thousands of single-nucleotide variants (SNVs) and hundreds of insertions and deletions (indels) (Schaefer et al. Nat. Methods 14, 547-548, 2017). Therefore, much effort is needed to increase the fidelity of CRISPR-Cas9 with regard to off-target mutation generation, especially in the clinical setting (Schaefer et al. Nat. Methods 14, 547-548, 2017). Recently, two works proposed that Cas9-guide RNA possesses more energy than needed for optimal recognition of its intended target sequence, thereby enabling cleavage at mismatched off-target sites (Slaymaker et al. Science 351, 84-88, 2016; Kleinstiver et al. Nature 529, 490-95, 2016). Based on the inactive structure of Cas9-sgRNA complex with a partial dsDNA target (Anders et al. Nature 513, 569-573, 2014), several high-fidelity Cas9 variants have been designed and validated for elimination of off-target effects, demonstrating the structure-guided Cas9 engineering as a robust strategy for specificity improvement (Slaymaker et al. Science 351, 84-88, 2016; Kleinstiver et al. Nature 529, 490-49, 2016). Given that the previous efforts were based on an inactive structure, there is still a need to explore the mechanistic details and structures involved in the t-DNA cleavage mechanism at the HNH domain.

A recent cryo-EM study provided some structures of precatalytic, postcatalytic, and product states of the active Cas9·sgRNA·DNA complex in the presence of Mg²⁺ ions (Zhu et al., Nature Structural & Molecular Biology 26, 679-685, 2019). However, the proposed catalytically competent structure (Protein Data Bank (PDB) entry 600Y (PDB ID: 600Y)) is missing several residues and the magnesium ions.

### SUMMARY OF THE INVENTION

Inventors studied the catalytic cleavage reaction of the t-DNA at the HNH domain of the Cas9 by using a recently discovered catalytic-active structure of this enzyme in complex with RNA and DNA by using classical molecular dynamics (MD), hybrid quantum mechanics/molecular mechanics, and a modified target DNA and have identified Cas9 variants that provide a solution to the off-target/fidelity problems associated with native and current Cas9 variants. Without wishing to be bound by theory, it is believed that the use of these additional variants alone or in combination with other variants results in a high fidelity Cas9 protein for use in genetic engineering methods.

Inventors have also designed a mismatched structure (MM5) with a C to G mismatch at the fifth position of the t-DNA's PAM region that can be used to better understand the impact of sg-RNA and t-DNA complementarity on the catalysis process.

Based on the MD results, the second-coordination shell water could also be considered the nucleophile in addition to the metal-bound water. Calculated QM/MM results show that the nucleophilic attack by the second-coordination shell water is not energetically feasible (with the reaction energy of 32.6 kcal mol⁻¹) which shows the structural effect of the t-DNA mismatch on the catalytic function of the Cas9. The present disclosure further provides the electrostatic potential (ESP) charges of the attacking water and its non-covalent interactions with the active site residues show that the reactant of the matched is more favorable than the MM5. In agreement with the QM/MM energy barriers and reaction energies for the matched and MM5, results of the energy decomposition analysis (EDA) show that the non-bonded intermolecular interactions between the Cas9 and the residues of the active site in the transition state (TS) and product of the matched are considerably more stabilizing than the MM5. This shows that the amino acid residues of the Cas9 have stabilizing contributions in the reactant-TS path, but this facilitating contribution is significantly larger in the matched structure. It was noticed that the introduction of the proximal mismatch (at the fifth position) in DNA causes conformational shifts that substantially reduce the population of the conformations around the catalytic active state, which may lead to a decrease in the rate constant observed in the kinetic experiments. Thus, the present disclosure provides a method to better understand Cas9 and to better prepare modifications and tools to produce a more effective Cas9 protein.

The present disclosure also provides methods of modifying a target nucleic acid, using an RNA-guided CRISPR-Cas effector protein of the present disclosure and a guide RNA. The present disclosure provides methods of modulating transcription of a target nucleic acid.

Remarkably, the concept described herein expands the mutation range and mutation types for Cas9. For instance, the residues beyond the previously identified DNA-binding regions can be considered for modifications. Hence, the residues of interest are no longer limited to the polar and positively charged types. In some embodiments here, the Cas9 variants contain alterations to the acidic residues, and also, the substitutions are not limited to alanine, depending on design needs. In certain aspects the substitution can be one or more of alanine (Ala, A), arginine (Arg, R), asparagine (Asn, N), aspartic acid (Asp, D), cysteine (Cys, C), glutamic acid (Glu, E), glutamine (Gln, Q), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), or valine (Val, V) in place of the native amino acid.

In certain embodiments, the Cas9 variant comprise one or two simultaneous mutations at the following positions of SEQ ID NO:1: Lys896 and/or Lys253. In certain embodiments, the Cas9 variant has a modification that comprises K896 and K253. In certain embodiments, the modified Cas9 protein has a modification that comprises K896. In certain embodiments, the modified Cas9 protein has a modification that comprises K253. In certain embodiments, the modifications include additional modifications.

In certain embodiments, the Cas9 variants comprise one, two, three, four, or five simultaneous mutations at the following positions of SEQ ID NO:1: Lys896; Arg820; Lys253; Arg400, and/or Lys855. In certain embodiments the modified Cas9 variants include, but not are limited to, the following combination of mutations: K896/R820; K896/K253; K896/R400; K896/K855; R820/K253; R820/R400; R820/K855; K253/R400; K253/K855; R400/K855; K896/R820/K253; K896/R820/R400; K896/R820/K855; R820/K253/R400; R820/K253/K855; K253/R400/K855; K253/R400/K896; R400/K855/K896; R400/K855/K820; K855/K896/K253; K896/R820/K253/R400; K820/K253/R400/K855; K253/R400/K855/K896; R400K855/K896/R820; K855/K896/R820/K253; K896/R820/K253/R400/K855.

Certain embodiments are directed to further modified or variant Cas9 proteins. The modified Cas9 protein comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 additional modifications, for a total of 2 or more modification, including one or more modification or variant corresponding to Thr58, Glu60, Glu223, Glu370, Glu371, Asp406, Glu396, Glu584, Asp585, Arg586, Arg765, Asn767, Arg778, Glu779, Gln807, Tyr812, Gln844, Ser845, Arg859, Lys263, Lys902, Arg864, Lys866, Lys918, Asn14, Lys268, Arg447, Tyr450, Asn497, Lys500, Lys526, Lys528, Lys558, Asn588, Arg661, Asn692, Gln695, Arg780, Arg783, Asn803, Gln805, Lys810, Asp829, Asn831, Arg832, Asp835, Lys848, Lys862, Arg925, Gln926, Lys929, His930, Lys961, Lys968, Tyr1013, Lys1031, Lys1244, or Lys1246 of SEQ ID NO:1. In certain aspects the modified Cas9 protein has at least two amino acid modifications. The modified Cas9 protein can further comprise one or more modification that includes modification of Asn14, Lys268, Glu370, Arg447, Tyr450, Asn497, Lys500, Lys526, Lys528, Lys558, Asn588, Arg661, Asn692, Gln695, Arg780, Arg783, Asn803, Gln805, Lys810, Tyr812, Asp829, Asn831, Arg832, Asp835, Gln844, Lys848, Lys862, Arg925, Gln926, Lys929, His930, Lys961, Lys968, Tyr1013, Lys1031, Lys1244, or Lys1246 corresponding to SEQ ID NO:1.

The modification can be any amino acid other than the amino acid present in a corresponding position in SEQ ID NO:1. In a further aspect the modification can be a substitution with an alanine, glycine, lysine, arginine, aspartic acid, or glutamic acid substitution.

The modified Cas9 protein can be coupled or fused with a heterologous polypeptide or peptide. In certain aspects the modified Cas9 protein can include a nuclear localization signal, a cell penetrating amino acid sequence, or an affinity tag.

In certain aspects the modified Cas9 protein is a modified Streptococcus pyogenes Cas9 protein.

In a further aspect the modified Cas9 protein can be 70, 75, 80, 85, 90, 95, 96, 97, 98, 99% identical to SEQ ID NO:1, while retaining at least some of the Cas9 function of the protein of SEQ ID NO:1. The modified Cas9 protein can have at least 20, 30, 40, 50, 60, 70, 80, 90% fewer off-target events as compared to non-modified Cas9. Furthermore, the modified Cas9 protein can cleave at least 60, 65, 70, 75, 80, 85, 90, 95, to 100%, including all values and ranges there between, of the target sites as compared to non-modified Cas9, thus maintaining sufficient activity. The modified Cas9 protein can have a frequency of off-site events that is at least 20, 30, 40, 50, 60, 70, 80, 90% lower than off-target events as compared to non-modified Cas9. Specificity (fidelity) and cleavage activity of Cas9 variant are quantified as compared with the wild type protein. A gRNA targets a specific gene sequence, therefore there are a certain number of known off-target sequences. The native Cas9/gRNA complex is able to cleave the target DNA and all the off-target DNA sequences. The modified Cas9 protein reduces the cleavage of the off-target DNA sequence. The specificity (fidelity) can be determined by measuring the number of off-target cleavage. The lower number of off-target site cleavages, the higher the specificity (fidelity). For example, if a designed Cas9 mutant yields cleavage only at 10% of the off-target sites compared to the wild type protein, meaning 90% fewer off-target events, the gene editing specificity can be regarded as improving by 90%. The on-target activities of Cas9 proteins can be assessed using the human cell-based enhanced GFP (EGFP) disruption assay. For example, the wild type Cas9 guided by a fully matched gRNA induces 90% EGFP disruption, a certain Cas9 variant exhibiting a disruption percentage around that value (80%, 95%, for example) is considered as possessing the wild-type or near wild-type cleavage efficiency. In certain aspects of the invention, the criterion of >70% of wild-type activity is used for screening potential Cas9 variants for subsequent tests on a whole-genome level.

Certain embodiments are directed to a fusion protein comprising the modified Cas9 protein fused to a heterologous peptide or protein, with an optional intervening linker.

Other embodiments are directed to an expression cassette encoding the modified Cas9 protein or fusion protein comprising the modified Cas9 protein.

Still other embodiments are directed to an expression vector comprising the expression cassette encoding the modified Cas9 protein or fusion protein comprising the modified Cas9 protein.

Certain embodiments are directed to a host cell expressing an expression cassette of the invention. In certain aspects the host cell is an isolated host cell or a host in culture.

Other embodiments are directed to a host cell comprising a modified Cas9 protein described herein.

Certain embodiments are directed to methods of using such a modified Cas9 protein. Certain aspects include methods of altering the genome of a cell, the method comprising expressing in the cell or contacting the cell with the modified Cas9 protein described herein. In a further aspect the modified Cas9 protein is linked to a guide RNA having a region complementary to a selected portion of the genome of the cell. The method resulting in the alteration of the genome of the cell.

Other embodiments are directed to methods of altering a double stranded DNA (dsDNA) molecule, the method comprising contacting the dsDNA molecule with the modified Cas9 protein described herein. The modified Cas9 protein can be linked to a guide RNA having a region complementary to a selected portion of the dsDNA molecule, resulting in the alteration of the dsDNA molecule.

Certain embodiments are directed to a target DNA strand comprising a C to G mismatch at a fifth position of a protospacer adjacent motif (PAM) as compared to a guide DNA strand. Certain embodiments are directed to a system containing a Cas9 protein, a guide RNA, and a target DNA strand containing a C to G mismatch at a fifth position of a protospacer adjacent motif (PAM) as compared to the guide RNA. In some instances, the Cas9 protein is a modified Cas9 protein as described herein. In some instances, the target DNA strand is contained in a double stranded DNA that further contains a non-target DNA strand. In some instances, the non-target DNA strand contains a C at the nucleotide corresponding to the fifth position of the protospacer adjacent motif (PAM) of the target DNA strand. Other embodiments are directed to methods of testing and/or modeling the system. In some instances, the Cas9 protein is tested and/or modeled. The testing and/or modeling may include, but is not limited to, kinetic testing, structure modeling, binding affinities, minimum energy paths, analysis of catalytically important residues, etc. Other embodiments are directed to contacting a Cas9 protein with the target DNA strand comprising a C to G mismatch at a fifth position of a protospacer adjacent motif (PAM) as compared to a guide DNA strand.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

The terms “polypeptide”, “protein”, and “peptide”, which are used interchangeably herein, refer to a polymer of the protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide”, and “peptide” are used interchangeably herein when referring to a gene product. Thus, exemplary polypeptides include gene products, homologs, orthologs, paralogs, fragments and other equivalents, variants, and analogs of the foregoing.

The term “variant” or “mutant” refers to an amino acid sequence that is different from the reference polypeptide by one or more amino acids, e.g., one or more amino acid substitutions. For example a modified or variant Cas9 polypeptide differs from wild-type Cas9 (e.g., SEQ ID NO: 1) by one or more amino acid substitutions, e.g., mutations.

“Polynucleotide,” synonymously referred to as “nucleic acid molecule” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, double-stranded, or a mixture of single- and double-stranded regions.

“Substantially similar” with respect to nucleic acid or amino acid sequences, means at least about 65% identity between two or more sequences. Preferably, the term refers to at least about 70% identity between two or more sequences, more preferably at least about 75% identity, more preferably at least about 80% identity, more preferably at least about 85% identity, more preferably at least about 90% identity, more preferably at least about 91% identity, more preferably at least about 92% identity, more preferably at least about 93% identity, more preferably at least about 94% identity, more preferably at least about 95% identity, more preferably at least about 96% identity, more preferably at least about 97% identity, more preferably at least about 98% identity, and more preferably at least about 99% or greater identity. Such identity can be determined using algorithms known in the art, such as the mBLAST algorithm.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated polypeptide refers to one that can be administered to a cell or a subject; in other words, the polypeptide may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

The term “providing” is used according to its ordinary meaning “to supply or furnish for use.” In some embodiments, the protein is provided directly by administering the protein, while in other embodiments, the protein is effectively provided by administering a nucleic acid that encodes the protein. In certain aspects the invention contemplates compositions comprising various combinations of nucleic acid, and/or peptides.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present invention can “comprise,” “consist essentially of,” or “consist of” particular ingredients, components, blends, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1C Schematic representation of the reaction mechanisms for the DNA cleavage at the HNH domain of Cas9 by (A) first-shell water coordinated to Mg²⁺ or hydrolysis by (B) second-shell water around Mg²⁺. (C) The initial model of Streptococcus pyogenes Cas9 (SpyCas9 or SpCas9) and the close-up of the HNH's active site. Three coordinated water molecules to the magnesium ion are not shown.

FIGS. 2A-2B. (A) Cryo-EM structure for Protein Data Bank entry 600Y and (B) an initial model of Cas9 showing all the domains of Cas9, DNA, and sgRNA. The root-mean-square deviation (RMSD) between Cas9 in these structures is 1.91 Å over 947 aligned residues with 96.8% sequences identity. Dash lines indicate the missing regions in (A). The scale bar on the left-hand side shows the specific domains in Cas9 (t-DNA, nt-DNA, and sgRNA are colored in magenta, yellow, and light blue respectively).

FIGS. 3A-3C. Results for 10 ns with a constant number of particles (N), temperature (T), and volume (V) (NVT) with restraint on the active site for Matched^{1 st shell} using AMBER's CPPTRAJ⁷² (CPPTRAJ) for both the approximate transition state (TS) and product state by (A) all atoms (active site), (B) backbone, and (C) residue.

FIGS. 4A-4C. The CPPTRAJ results for 10 ns of NVT with restraint on the active site for MM5 for both the approximate transition state (TS) and product state by (A) all atoms (active site), (B) backbone, and (C) residue.

FIGS. 5A-5B. (A) Variation in the Root-mean-square deviation (RMSD) of Cα atoms of Cas9 protein backbone during the simulation for Matched and MM5. The error bars represent the standard deviation from two independent simulations. (B) Root-mean-square fluctuation (RMSF) of each residue averaged over the 50-150 ns for the Matched and MM5 systems. Here, the RMSF of each residue is averaged over two independent simulations for each system.

FIGS. 6A-6D. DNA, sgRNA, and protein interactions for (A) matched-Cas9 and (B) MM5-Cas9 focusing HNH catalytic site and PAM (NGG) region. (C) and (D) show different views of the matched and MM5 zooming out the PAM distal end and RuvC region interactions. The t-DNA, nt-DNA, and sgRNA are colored differently from each other. The two nuclease domains of Cas9, HNH, and RuvC are shown in white and grey.

FIGS. 7A-7B. (A) Binding enthalpies (kcal mol⁻¹) between the sgRNA+Cas9 (receptor) and the DNA (ligand) for Matched and MM5 calculated based on the molecular mechanics/generalized Born surface area (MM/GBSA) approach on two independent Molecular Dynamics (MD) simulations. (B) Graphical representation of the relative binding strengths for Matched and MM5 considering the average value for Matched as the 100% binding strength.

FIGS. 8A-8B. Dynamics cross-correlation maps from normal mode analysis. These plots show correlated motions of the backbone atoms between all residue pairs in (A) Matched and (B) MM5 considering CA atoms of residues in Cas9. The scale color bar on the bottom indicates the extent of the correlation. Moving toward 1.0 indicates a high level of correlated motions (residue pairs move together in the same direction), while moving toward-1.0 indicates anti-correlated motions (residue pairs move together in the opposite direction).

FIG. 9. Orientations of the active site's residues in the selected representatives from the clustering for the Matched.

FIG. 10. Orientations of the active site's residues in the selected representatives from the clustering for the MM5.

FIGS. 11A-11C. Active site's structure for (A) Matched^{1 st shell}, (B) Matched^{2 nd shell}, and (C) MM5 optimized at ωB97X-D/6-31G** level of theory with AMBER ff14SB Force Field. The fifth nucleotide from the PAM region, dC(+5) on the t-DNA, highlights the main difference between matched and MM5. Residues V838 and 1841 of the active site are not shown for more clarity.

FIGS. 12A-12C. The optimized geometries of reactant (R), approximate transition state (TS), and the product (P) of the cleavage reaction at the HNH domain of (A) Matched^{1 st shell} and (B) MM5. The distances between the atoms involved in the reaction are shown in dotted lines. The nucleophilic water and the Mg²⁺ are shown in ball-and-sticks, while all the other atoms are in licorice. The chain of the protein, t-DNA, and sg-RNA are shown in different shades of grey. (C) The minimum energy path for the cleavage reaction at the HNH domain of the Matched^{1 st shell} and the MM5 is modeled by the quadratic string model (QSM). QM/MM optimization energies were calculated at ωB97X-D/6-31G** level of theory with AMBER ff14SB Force Field.

FIG. 13. The optimized geometries and the related QM/MM energies (kcal mol⁻¹) of the reactant and product of the cleavage reaction at the HNH domain of the WT2nd shell calculated at ωB97X-D/6-31G** level of theory with AMBER ff14SB Force Field. The critical distances with the corresponded values are shown in dotted lines. The nucleophilic water and the Mg (II) are shown in ball-and-sticks, while all the other atoms are in licorice. The chain of the protein, t-DNA, and sg-RNA are shown in different shaded ribbons. Hydrogen atoms of the amino acids and the nucleotides are not presented for more clarity.

FIG. 14A-14B. (A) Superposition of the Matched^{1 st shell} and MM5 structures' active sites optimized by the QM/MM (RMSD is 2.16 Å over 118 aligned residues). (B). Superposition of the Matched^{1 st shell} and Matched^{2 nd shell} structures' active sites optimized by the QM/MM (RMSD is 1.99 Å over 121 aligned residues).

FIGS. 15A-15C. Calculated ESP charges for the nucleophilic water and the plots of the non-covalent interactions between the nucleophilic water and the surrounding residues in the reactants of (A) Matched^{1 st shell}, (B) Matched^{2 nd shell}, and (C) the MM5. The isovalue for the non-covalent interactions (NCI) visualization is 0.4 with the color scale of −0.05 au<sign (λ₂)ρ<0.05 au. The ESP charges are extracted from the optimized structures of the reactants at ωB97X-D/6-31G** level of theory with AMBER ff14SB Force Field. The nucleophilic water and the Mg²⁺ are shown in ball-and-sticks, while all the other atoms are in licorice. Hydrogen atoms of the amino acids and the nucleotides are not presented for more clarity except for the ζ-hydrogens of K862 in (B).

FIGS. 16A-16B Calculated

$$Δ {E}_{Intermol , Interact}^{T S} , - Δ {E}_{Intermol , Interact .}^{Reactant}$$

for (Up) the Matched^{1 st shell} and (Down) the MM5. The stabilizing and de-stabilizing residues with |ΔΔE_{Intermol.Interact}|larger than 10 kcal mol⁻¹ (for Matched) and 5 kcal mol⁻¹ (for MM5) are shown in different shades of grey. The vertical grey lines in each graph show the location of the amino acid residues of the active site.

FIGS. 17A-17B Calculated

$$Δ {E}_{Intermol , Interact}^{Product} , - Δ {E}_{Intermol , Interact .}^{Reactant}$$

for (Up) the Matched^{1 st shell} and (Down) the MM5. The stabilizing and de-stabilizing residues with |ΔΔE_{Intermol.Interact.}|larger than 10 kcal mol⁻¹ (for Matched) and 5 kcal mol⁻¹ (for MM5) are shown in different shades of grey. The vertical grey lines in each graph show the location of the amino acid residues of the active site.

FIG. 18. The total intermolecular contributions (E_{intermol.Interact.}=E_{van der Waals}+E_{coulomb}) between the active site and the protein environment in the reactant of the (Up) Matched^{1st shell} and (middle) MM5. (Down) The difference of the total intermolecular contribution energies ΔΔE_{Intermol.Interact.} between the reactants of the MM5 and Matched^{1 st shell}. The vertical grey lines in each graph show the location of the amino acid residues of the active site.

FIG. 19. The total intermolecular contributions (E_{intermol.Interact.}=E_{van der Waals}+E_{coulomb}) between the active site and the protein environment in the product of the (Up) Matched^{1 st shell} and (middle) MM5. (Down) The difference of the total intermolecular contribution energies ΔΔE_{Intermol.Interact.} between the products of the MM5 and Matched^{1 st shell}. The vertical grey lines in each graph show the location of the amino acid residues of the active site.

FIGS. 20A-20B Potent candidate residues with allosteric effects proposed by the EDA calculations. Candidate residues are shown in licorice with corresponding residue names and numbers in bold text. The active site's residues are displayed in ball-and-stick, and the residue names and numbers are given in the italic text. The hydrogen atoms are not shown for more clarity. (B) The list of the residues with different allosteric effects on the matched and the MM5. The threshold for the selection is ΔE_{intermol.Interact.}≥5 kcal mol⁻¹ for a residue in matched and ΔΔE_{Intermol.Interact.}≤5 kcal mol⁻¹ for the same residue in the MM5 system.

FIG. 21. The candidate residue R780 (found from EDA analysis) showing its interaction with the mismatched region of t-DNA and corresponding interaction in the matched system.

FIG. 22. The candidate residues R859, R832, and R780 (found from EDA analysis) showing their interaction with the mismatched region of t-DNA and corresponding interaction in the matched system.

FIG. 23. The candidate residues K896 and R820 (found from EDA analysis) showing their interaction with the mismatched region of t-DNA and corresponding interaction in the matched system.

FIG. 24. The candidate residue K253 (found from EDA analysis) showing its interaction with the mismatched region of t-DNA and corresponding interaction in the matched system.

FIG. 25. The candidate residue R661 (found from EDA analysis) showing its interaction with the mismatched region of t-DNA and corresponding interaction in the matched system.

FIG. 26 geometry of the reactant's active site in Matched^{1st shell}, MM5, and Matched^{2nd shell}.

### DETAILED DESCRIPTION OF THE INVENTION

Inventors studied the catalytic cleavage reaction of the t-DNA at the HNH domain of the Cas9 by using a recently discovered catalytic-active structure of this enzyme in complex with RNA and DNA by using classical molecular dynamics (MD), hybrid quantum mechanics/molecular mechanics, and a modified target DNA and have identified Cas9 variants that provide a solution to the off-target/fidelity problems associated with native and current Cas9 variants. Without wishing to be bound by theory, it is believed that the use of these additional variants alone or in combination with other variants results in a high fidelity Cas9 protein for use in genetic engineering methods. Inventors have also designed a mismatched structure (MM5) with a C to G mismatch at the fifth position of the t-DNA's PAM region that can be used to better understand the impact of sg-RNA and t-DNA complementarity on the catalysis process.

The bacterial CRISPR-Cas9 system has been adapted as a powerful and versatile genome-editing toolbox. The system holds immense promise for future therapeutic applications. Despite recent advances in Cas9 structure/function, little is known on the catalytic state of Cas9 HNH nuclease domain and it remains elusive how the divalent metal ions affect the HNH domain conformational transition. A deep understanding of Cas9 activation and cleavage mechanism can enable further optimization of Cas9-based genome-editing specificity and efficiency.

The new structural information here can be exploited to rationally design more Cas9 variants with improved specificity. After careful inspection of the locations of the identified residues and their interactions within the whole complex, the inventors suggest several sites to be mutated. Further integration with previously screened candidate sites, it is believed that different versions of high-fidelity Cas9 mutants could be customized specially for minimizing the off-target effects occurring at the PAM proximal or distal ends, or even at the non-standard repetitive sites. It would make more sense, as there is no one versatile Cas9 nuclease capable of eliminating all sorts of off-target cleavage.

Activities of modified Cas9 polypeptides can be assessed in a bacterial cell-based system with survival percentages between 50-100% usually indicating robust cleavage, whereas 0% survival indicated that the enzyme had been functionally compromised.

To further determine whether the Cas9 variants described herein function efficiently in human cells, modified proteins can be tested using a human cell-based EGFP-disruption assays. In this assay, successful cleavage of a target site in the coding sequence of a single integrated, constitutively expressed EGFP gene leads to the induction of mutations and disruption of EGFP activity, which can be quantitatively assessed by flow cytometry (see, for example, Reyon et al., Nat Biotechnol. 30 (5): 460-5, 2012).

All of the variants described herein can be incorporated into existing vectors Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria or other host cell expression system.

The term “functionally equivalent codon” is used herein to refer to codons that encode the same amino acid, such as the six codons for arginine or serine, and also refers to codons that encode biologically equivalent amino acids. Codons include: Alanine (Ala, A) GCA, GCC, GCG, and GCU; Cysteine (Cys, C) UGC and UGU; Aspartic acid (Asp, D) GAC and GAU; Glutamic acid (Glu, E) GAA and GAG; Phenylalanine (Phe, F) UUC and UUU; Glycine (Gly, G) GGA, GGC, GGG, and GGU; Histidine (His, H) CAC and CAU; Isoleucine (Ile, I) AUA, AUC, and AUU; Lysine (Lys, K) AAA and AAG; Leucine (Leu, L) UUA, UUG, CUA, CUC, CUG, and CUU; Methionine (Met, M) AUG; Asparagine (Asn, N) AAC and AAU; Proline (Pro, P) CCA, CCC, CCG, and CCU; Glutamine (Gln, Q) CAA and CAG; Arginine (Arg, R) AGA, AGG, CGA, CGC, CGG, and CGU; Serine (Ser, S) AGC, AGU, UCA, UCC, UCG, and UCU; Threonine (Thr, T) ACA, ACC, ACG, and ACU; Valine (Val, V) GUA, GUC, GUG, and GUU; Tryptophan (Trp, W) UGG; and Tyrosine (Tyr, Y) UAC and UAU.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, and the like.

It also is understood in the art that the substitution of like amino acids can be made effectively on the basis of hydrophilicity. It is understood that an amino acid can be substituted for another having a similar hydrophilicity value and still produce a biologically equivalent protein.

As outlined above, amino acid substitutions generally are based on the relative similarity of the amino acid side-chain substituents, for example, their hydrophobicity, hydrophilicity, charge, size, and the like. Examples of substitutions that take into consideration the various foregoing characteristics are well known and include: arginine and lysine; glutamate and aspartate; serine and threonine; glutamine and asparagine; and valine, leucine and isoleucine.

Embodiments involve polypeptides, peptides, proteins and fragments thereof for use in various aspects described herein. In specific embodiments, all or part of proteins described herein can also be synthesized in solution or on a solid support in accordance with conventional techniques. Various automatic synthesizers are commercially available and can be used in accordance with known protocols. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions.

Also included are fusion proteins. Embodiments can include individual fusion proteins as a fusion protein with heterologous sequences such as a provider of purification tags, for example: β-galactosidase, glutathione-S-transferase, green fluorescent proteins (GFP), epitope tags such as FLAG, myc tag, or polyhistidine.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. As used herein an amino acid designated as “X” refers to any amino acid residue. However, when in the context of an amino acid substitution it is to be understood that “X” followed by a number refers to an amino acid residue at a particular location in a reference sequence.

As used herein, an amino acid residue of an amino acid sequence of interest that “corresponds to” or is “corresponding to” or in “correspondence with” an amino acid residue of a reference amino acid sequence indicates that the amino acid residue of the sequence of interest is at a location homologous or equivalent to an enumerated residue in the reference amino acid sequence. One skilled in the art can determine whether a particular amino acid residue position in a polypeptide corresponds to that of a homologous reference sequence. For example, the sequence of a modified or related Cas9 protein can be aligned with that of a reference sequence (e.g., SEQ ID NO: 1 using known techniques (e.g., basic local alignment search tool (BLAST), ClustalW2, Structure based sequences alignment program (STRAP), or the like). In addition, crystal structure coordinates of a reference sequence may be used as an aid in determining a homologous polypeptide residue's three dimensional structure. Using such methods, the amino acid residues of a polypeptide can be numbered according to the corresponding amino acid residue position numbering of the reference sequence. For example, the amino acid sequence of SEQ ID NO: 1 may be used for determining amino acid residue position numbering of each amino acid residue of a variant of interest.

The term “identical” in the context of two nucleic acids or polypeptide sequences refers to the residues in the two sequences that are the same when aligned for maximum correspondence, as measured using one of the following sequence comparison or analysis algorithms.

The percent sequence identity between a reference sequence and a test sequence of interest may be readily determined by one skilled in the art. The percent identity shared by polynucleotide or polypeptide sequences is determined by direct comparison of the sequence information between the molecules by aligning the sequences and determining the identity by methods known in the art. An example of an algorithm that is suitable for determining sequence similarity is the BLAST algorithm, (see Altschul, et al., J. Mol. Biol., 215:403-410 [1990]). Software for performing BLAST analyses is publicly available through the National Center for Biotechnology Information. This algorithm involves first identifying high scoring sequence pairs (HSPs) by identifying short words of length W in the query sequence that either match or satisfy some positive-valued threshold score T when aligned with a word of the same length in a database sequence. These initial neighborhood word hits act as starting points to find longer HSPs containing them. The word hits are expanded in both directions along each of the two sequences being compared for as far as the cumulative alignment score can be increased. Extension of the word hits is stopped when: the cumulative alignment score falls off by the quantity X from a maximum achieved value; the cumulative score goes to zero or below; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915 [1992]) alignments (B) of 50, expectation (E) of 10, M′5, N′-4, and a comparison of both strands.

The BLAST algorithm then performs a statistical analysis of the similarity between two sequences (see, e.g., Karlin and Altschul, supra). One measure of similarity provided by the BLAST algorithm is the smallest sum probability (P(N)), which provides an indication of the probability by which a match between two nucleotide or amino acid sequences would occur by chance.

Percent “identical” or “identity” in the context of two or more nucleic acid or polypeptide sequences refers to two or more sequences that are the same or have a specified percentage of nucleic acid residues or amino acid residues, respectively, that are the same, when compared and aligned for maximum similarity, as determined using a sequence comparison algorithm or by visual inspection. “Percent sequence identity” or “% identity” or “% sequence identity or “% amino acid sequence identity” of a subject amino acid sequence to a reference amino acid sequence means that the subject amino acid sequence is identical (e.g., on an amino acid-by-amino acid basis) by a specified percentage to the reference amino acid sequence over a comparison length when the sequences are optimally aligned. Thus, 80% amino acid sequence identity or 80% identity with respect to two amino acid sequences means that 80% of the amino acid residues in two optimally aligned amino acid sequences are identical.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### A. Example 1

### 1. Computational Methods

i. Molecular Dynamics (MD) Simulations

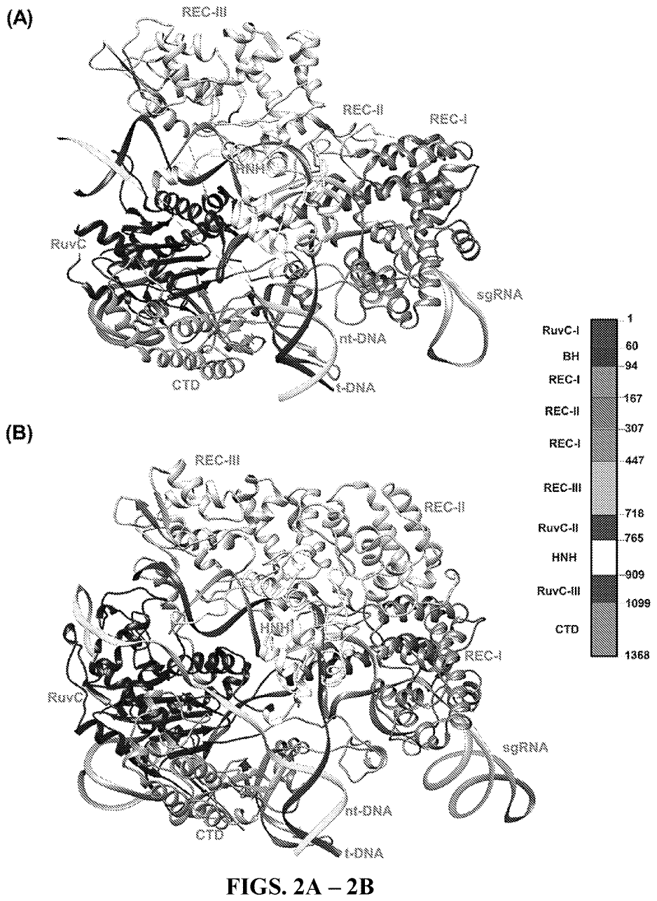

Structural Model: A stepwise approach was used to build the initial model due to some missing residues in the recently discovered active-state cryo-EM structure of SpyCas9. (PDB ID: 600Y58, FIG. 2). The missing regions of the Cas9 protein are residues: 175-310 (REC2), 713-717, and 1002-1075 (RuvC-III), unresolved nucleotides of the nt-DNA, and the absence of metal ions in the nuclease domains. A structure generated from a previous simulation study,⁵⁷ which achieved the HNH precatalytic/active state, was utilized as the starting point. The mentioned structure was based on the most complete X-ray structure of Cas9 in complex with RNA and DNA (PDB ID: 5F9R).⁵⁸ In a previous study,⁵⁷ Mg²⁺ ion was added to the HNH catalytic center, and nt-DNA (present in 5F9R) was removed to achieve the HNH precatalytic/active state at a shorter time scale. The missing nt-DNA was included in the current study by a superposition with crystal structure 5F9R, and the unresolved portion was added manually. To retain the conformation of the RuvC catalytic center comparable to that of the cryo-EM structure (600Y), the coordinates of H983 and residues 3-12 were replaced by the corresponding regions from the cryo-EM structure. In this structure, the positions of two Mg²⁺ ions of the RuvC domain were derived from the X-ray structure of CRISPR-Cas9, 4CMQ⁵⁹ solved in complex with Mn²⁺ ions. The final model, which is used as the starting point for the MD simulations, is shown in FIG. 2B. Furthermore, to investigate the impact of sgRNA and t-DNA complementarity on the catalysis process, a mismatched system called MM5 was created by mutating the fifth position nucleotide downstream of the PAM on t-DNA (C to G). The corresponding nt-DNA nucleotide (G to C) was also mutated to maintain the complementarity between the t-DNA and nt-DNA.

MD setup: The LEaP module in AMBER1860 was used to add the hydrogen atoms, neutralize the system with the corresponding number of required counterions, and solvate the structure in a rectangular box filled with TIP3P⁶¹ water extending at least 12 Å from the complex surface. The ff14SB,⁶² OL156,⁶³ and OL364 force fields were used to describe the molecular characteristics of the protein, DNA, and RNA, respectively. The MD simulations were done via AMBER18's pmemd.cuda.⁶⁵ Each system was minimized for 10,000 cycles by employing the steepest descent algorithm for the first 1000 cycles and conjugated gradient algorithm for the remaining cycles with restraints on the solute's heavy atoms. In the next step, each system was heated to 310 K using Langevin dynamics⁶⁶⁻⁶⁸ with a collision frequency of 2 ps⁻¹ followed by equilibration for 1000 ps in an NPT ensemble, keeping lowered restraints on the heavy atoms of solute. Lastly, the production calculations were performed on an unrestrained system in the NPT ensemble. All bonds involving hydrogen atoms were treated using SHAKE,⁶⁹ and long-range Coulomb interactions⁷⁰ were handled with the smooth particle mesh Ewald method⁷¹ using a 10 Å cutoff for non-bonded interactions. Individual simulations were run in duplicate, each for at least 200 ns with an integration time-step of 2 fs, and trajectories were saved at every 2 ps.

Structural analysis: AMBER's CPPTRAJ⁷² program was used to calculate the RMSD, RMSF, correlation matrices, and clustering analyses. To perform the clustering analysis, 100,000 trajectories in the range of 50 to 150 ns-maintaining the catalytically competent HNH domain—from two replicates of the matched and MM5 were used for a multi-dimensional analysis via the k-means algorithm⁷³ implemented in AMBER's CPPTRAJ. Each dimension of this analysis on the active site corresponds to a distance between the Mg²⁺ ion and its coordinated residues D839, H840, N863, and dT(+4). Ten clusters, each containing three representatives, were initially obtained to find the closest representatives to the centroids of each cluster in the matched and the MM5 systems. In the next step, four clusters for the matched and one for the MM5 with the highest population abundance and the best orientations of the active site's residues involved in the cleavage reaction were selected for further QM/MM optimizations.

ii. MM/GBSA Calculations

The molecular mechanics/generalized Born surface area (MM/GBSA) 74-76 method was employed based on the “single-trajectory” protocol⁷⁷ to calculate the binding enthalpies for the matched and MM5 systems via two different approaches. In the first approach, DNA and the sgRNA+Cas9 were considered the ligand and receptor, respectively. In the second one, the HNH's active site is regarded as the ligand (residues: 838-841, 863, 1493-1495, and 1541), while the rest of the system is considered as the receptor (residues: 1-837, 842-862, 864-1492, 1496-1540, and 1542-1543). The last 10,000 frames of MD for both replicates of each structure were used for the binding enthalpy calculations. The MM/GBSA calculations were performed via the MMPBSA.py internal module of AmberTools.⁷⁸ In addition to the computational efficiency of MM/GBSA, several studies have shown that this method results in comparable or even more accurate data in ranking ligand affinities compared to the molecular mechanics/Poisson-Boltzmann surface area (MM/PBSA).⁷⁹⁻⁸² To correct the nonpolar contribution to the solvation free energy, default values of the offset and surface tension were used, and the salt concentration in the GB equation was set to 150 mM. The entropic contributions were not added to the calculations due to the high computational cost and the potential convergence problem. However, based on many previous studies, MM/GB (PB) SA can achieve satisfactory accuracy in comparing relative ligand binding affinities, especially in cases where the ligands are very similar.⁷⁹⁻⁸⁴ Since the only difference between the matched and MM5 is a G to C mutation in the fifth position of the ligand (t-DNA), the entropic effect is not expected to be highly determinant.

iii. QM/MM Calculations

LICHEM^{85, 86} was used in combination with Gaussian16⁸⁷ and TINKER⁸⁸ for all QM/MM simulations of the matched and MM5 systems. The ωB97X-D/6-31G (d,p)^{89, 90} level of theory and the AMBER ff14SB force field were employed for the QM region and the MM environment. The QM/MM long-range electrostatic correction (QM/MM-LREC) method⁹¹ was used with a 25 Å cutoff for the QM subsystem coupled with the particle mesh Ewald⁷⁰ (PME) method for the MM calculations. The QM subsystem for both systems includes Mg²⁺, coordinated water molecules, V838, D839, H840, 1841, N863, dG(+3), and dT(+4). Residues dC(+5) or its mutation dG(+5) were added to the QM subsystem in the matched and MM5 systems, respectively. The nucleophilic water in the second shell around Mg²⁺ was also included in the QM subsystem of the matched 2nd shell system. The remaining residues and all solvent molecules are described by the AMBER ff14SB potential. The pseudobond approach⁹² was also applied to treat the covalent boundaries for the nucleic acid, e.g., dG(+3) and dC(+5)/dG(+5), and protein residues (V838, 1841, and N863) of the QM subsystem. In all cases, the optimizations were carried out using the iterative QM/MM optimization protocol implemented in LICHEM,^{85, 86} where all atoms in the MM subsystem within a radius of 25 Å from the center of the active site (Mg²⁺) were optimized and the rest were kept frozen.

After optimizing all the selected representatives of the matched and MM5, the one with the lowest QM/MM optimization energy in each structure was considered the most stable reactant and was used to design the initial structure of the product. The simulated products were then used for the further QM/MM calculations at the same level of theory. Based on the optimized reactant and product structures of each system (matched and the MM5), the potential energy surface of the reaction path was tried to be obtained and compared using the quadratic string model (QSM) combined with a restrained MM procedure as implemented in LICHEM.⁸⁶ The restraint on the MM environment started at 50 kcal mol⁻¹ Å⁻² and gradually decreased to zero. A chain of fourteen beads between the reactant (bead 0) and the product (bead 15), resulting in sixteen beads, was employed for guessing the reaction path.

Non-covalent interactions (NCI) were analyzed using the promolecular density method⁹³ implemented in the Multiwfn⁹⁴ code, using a cubic grid of 200 au. This analysis gives a qualitative view into the chemical bonding and weak noncovalent interactions between the molecule(s) of interest and the surrounding residues, based on the relationship between the electronic density and the reduced density gradient in regions of low electron density. The isovalue of 0.4 au with the color scale of −0.05 au<sign (λ₂)ρ<0.05 au was used to illustrate the NCI surfaces. The specific RGB colors of the NCI surfaces showed the strength and characteristics of the interactions. Red surfaces showed repulsive interactions, while green and blue surfaces represent weak and strong interactions like van der Waals and hydrogen bonds.

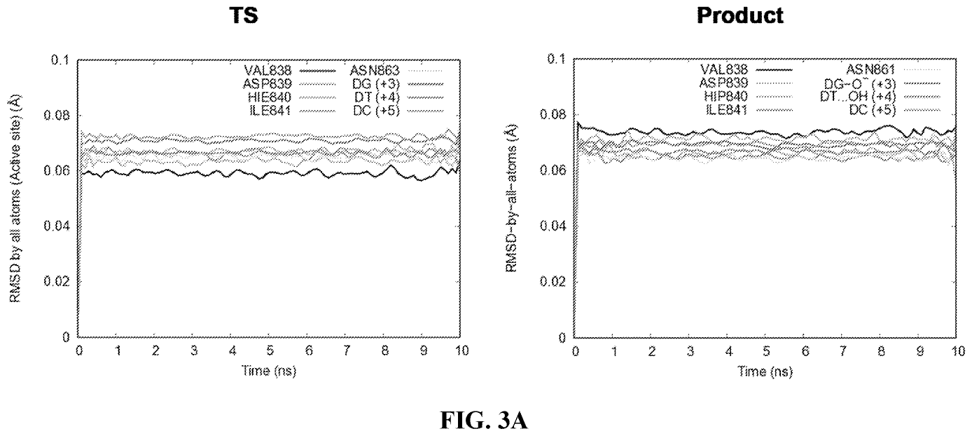

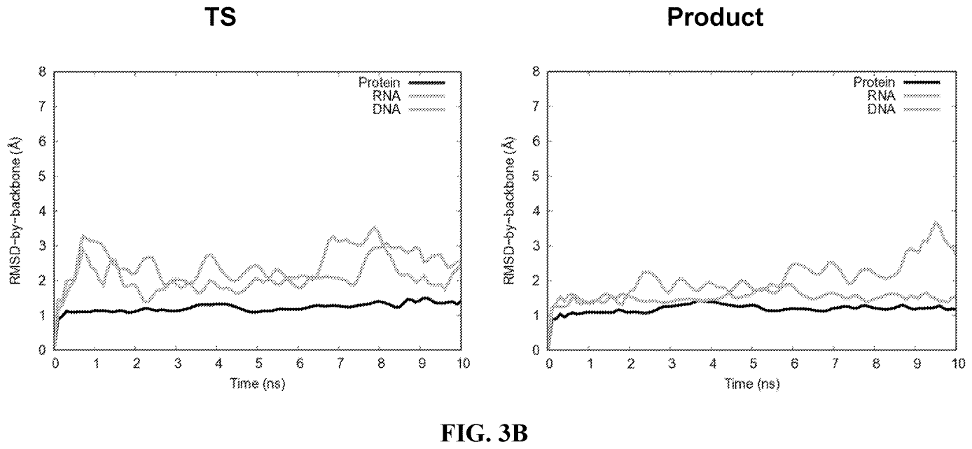

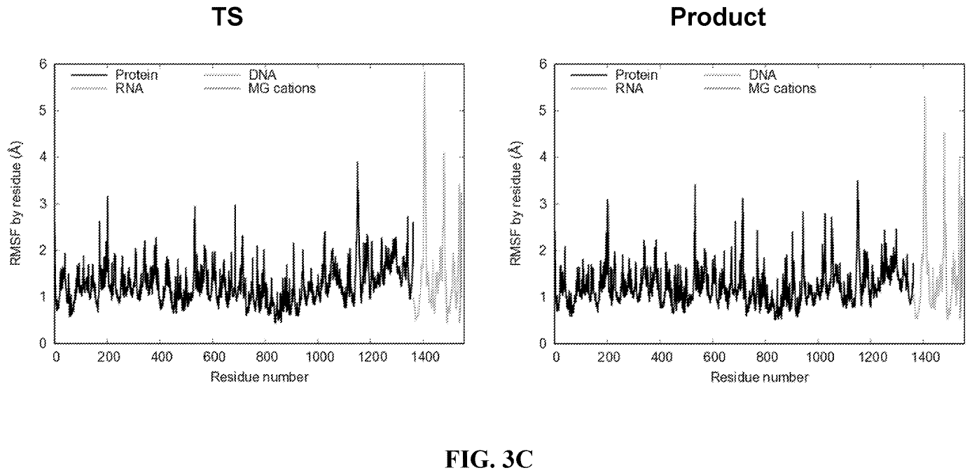

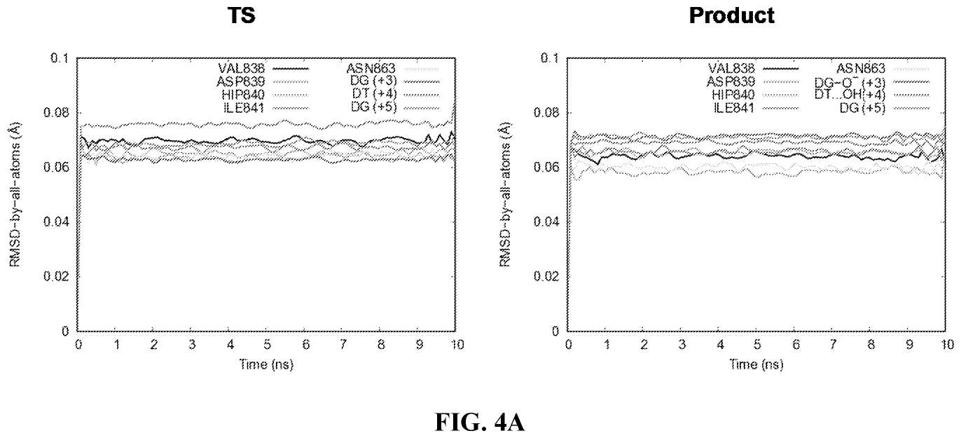

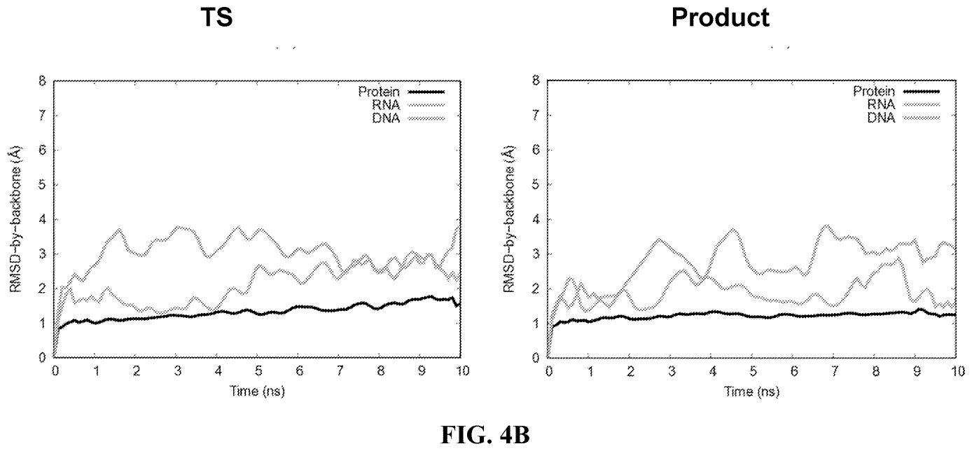

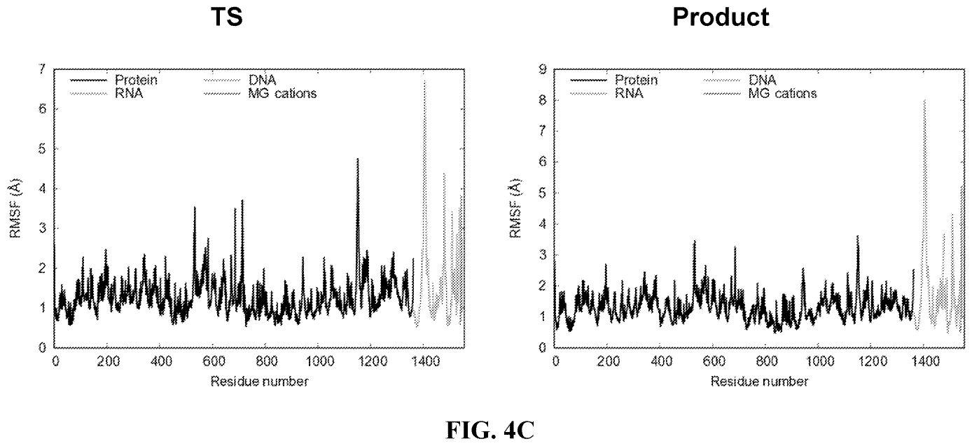

The QM/MM-optimized structures of the reactant, product, and approximate TS were used for further MD simulations with restraints on the QM region to perform the EDA analysis. In all cases, in addition to the optimized coordinates, the calculated ESP charges of the QM region (QM atoms and pseudobond atoms) were employed and transferred to the new topology files by the AMBER's ParmEd module.⁹⁵ For the approximate TS structures, the optimized coordinates from the QSM calculations were used for the MD simulations. Transient non-standard residues, dG-O⁻(+3) and dT . . . OH(+4), which form during the phosphodiester bond cleavage at the TS, were initially parameterized by the R.E.D. Server⁹⁶⁻⁹⁹ and the missing bonded parameters were added by ANTECHAMBER.^{100, 101} For the product structures, the dT-OH(+4), which forms after the DNA cleavage, was parameterized using the R.E.D. Server. In the next step, the LEaP module was employed to generate the coordinate and topology files of the TS and products for the MD simulations. Lastly, 10 ns of MD simulation with 200 kcal mol⁻¹ Å⁻² restraint on the QM atoms was performed at a temperature of 310 K in the NVT ensemble. All bonds involving hydrogen atoms were treated using SHAKE. Long-range Coulomb interactions were handled with the smooth particle mesh Ewald method using a 10 Å cutoff for non-bonded interactions. The CPPTRAJ module was used to analyze the RMSD and RMSF values of the MD simulations to monitor the stability of the TS and the product in matched and the MM5 (FIGS. 3 and 4). All the 2500 frames of these 10 ns of MD on the matched and the MM5's products were also employed for further binding enthalpies calculation via the MM/GBSA method as explained in the second approach of the “MM/GBSA calculations” section.

EDA implemented in an in-house Fortran90 program was employed for the mentioned structures to calculate the non-bonded inter-molecular interaction energies along the cleavage reaction path.¹⁰²⁻¹⁰⁴ This analysis was performed on the MD-simulated trajectories by considering the changes in Coulomb and van der Waals interaction energies between the QM subsystem and the residues of the MM region when the system goes from the reactant to the transition state and the product. This difference in the non-bonded inter-molecular interaction energy ΔΔE_{Intermol.Interact.} can be calculated as:

$$Δ Δ {E}_{Intermol . Interact .} = Δ {E}_{Intermol . Interact .}^{TS / Product} - Δ {E}_{Intermol . Interact .}^{Reactant} \qquad Eqn . 1$$

where

$$Δ {E}_{Intermol . Interact .}^{TS / Product}$$

represents the difference between the non-bonded inter-molecular interactions of the TS or product and

$$Δ {E}_{Intermol . Interact .}^{Reactant}$$

represents the same values for the reactant. This analysis gives a qualitative assessment of the catalytic role of amino acid residues surrounding the active site, showing the stabilizing and destabilizing residues affecting the catalytic reaction, and was used to analyze the QM/MM and MD simulations of several protein systems.¹⁰⁵⁻¹¹¹ The UCSF Chimera,¹¹² VMD,¹¹³ and GaussView 6.1¹¹⁴ programs were used for rendering the images.

### 2. Results and Discussion

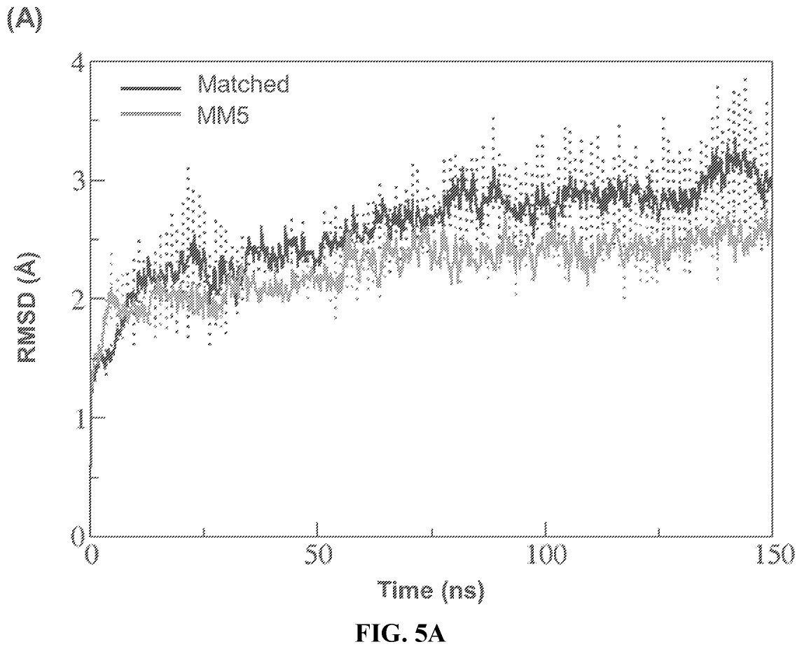

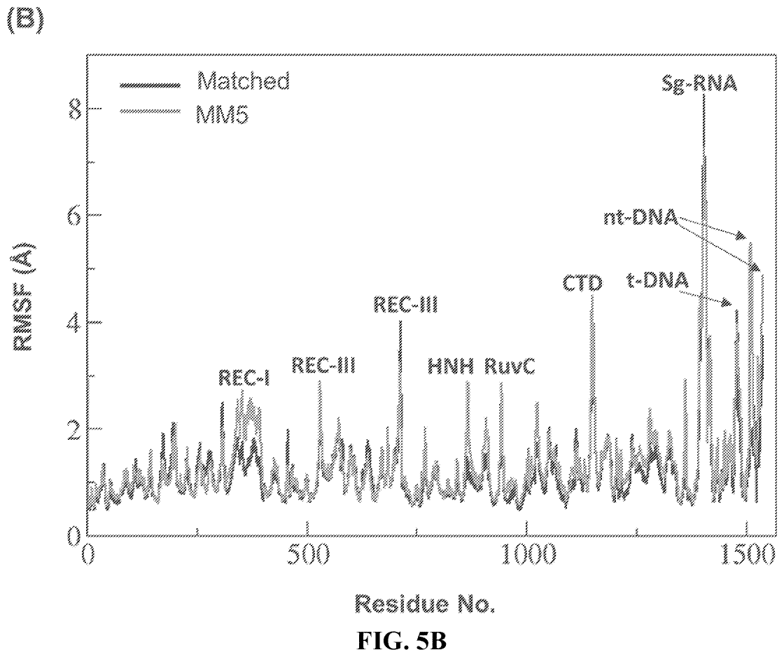

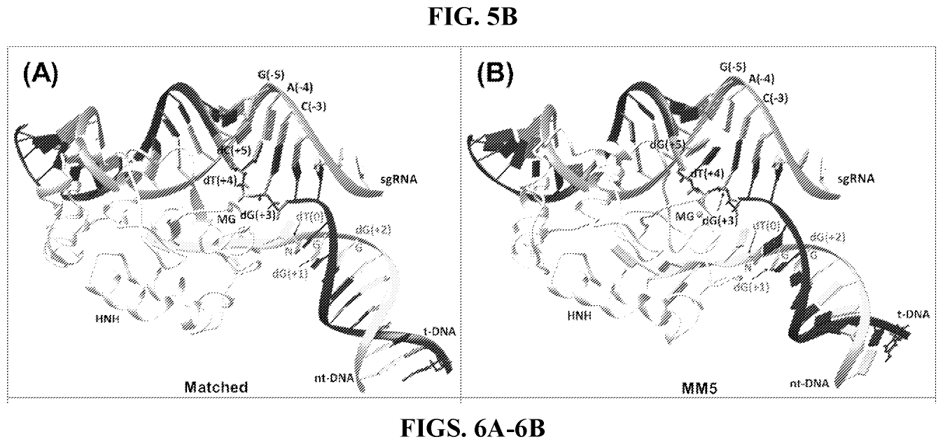

Mismatched and matched systems maintain stable conformations for HNH catalytic state. All-atom MD simulations in an aqueous solution were performed to obtain the initial conformation of the DNA and sgRNA bound Cas9 with catalytically active HNH domain for matched and MM5 systems (in two replicates). Throughout the simulations, the distance between the nitrogen atom of H840 and the scissile phosphate (OP1-dT(+4)) was maintained between 5.61 Å and 5.65 Å for matched and MM5, respectively. Hence, this range of the MD simulation was considered for further analysis, representing a suitable coordination geometry of the DNA substrate and the active site residues with Mg²⁺ ion in the HNH domain. The time-dependent root-mean-square deviation (RMSD) plots for the alpha carbon (Cα) atoms of the Cas9 protein for the matched and MM5 systems are shown in FIG. 5A. The RMSD values converged within 50 ns for matched and MM5, indicating that systems have reached a stable state. However, the RMSD of the backbone of the Cas9 is slightly lower for the MM5 system than that of matched, suggesting that Cas9 protein explores alternative dynamics and conformation in the presence of mismatched DNA. Moreover, it was found that the introduction of proximal mismatched DNA has a distinct effect on the flexibility in the different regions of the Cas9·sgRNA.DNA complex (FIG. 5B). It can also be observed in FIG. 5B that various regions of Cas9, e.g., REC-I, REC-III, HNH, RuvC, and CTD have higher flexibility in MM5 than in the matched system.

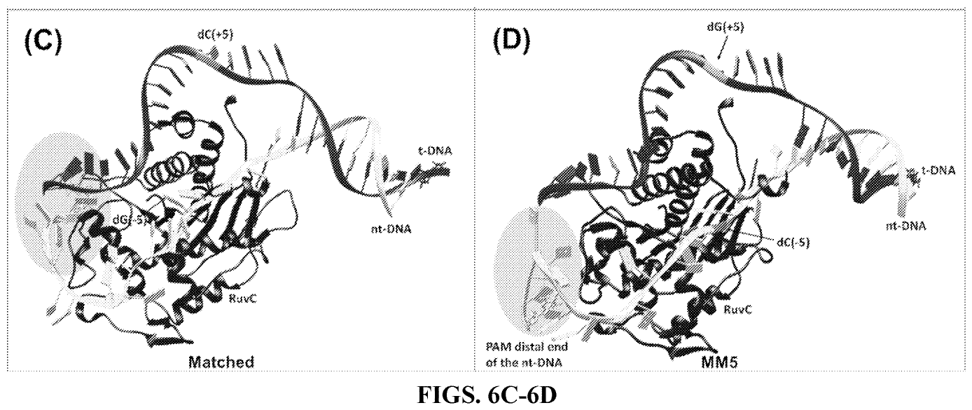

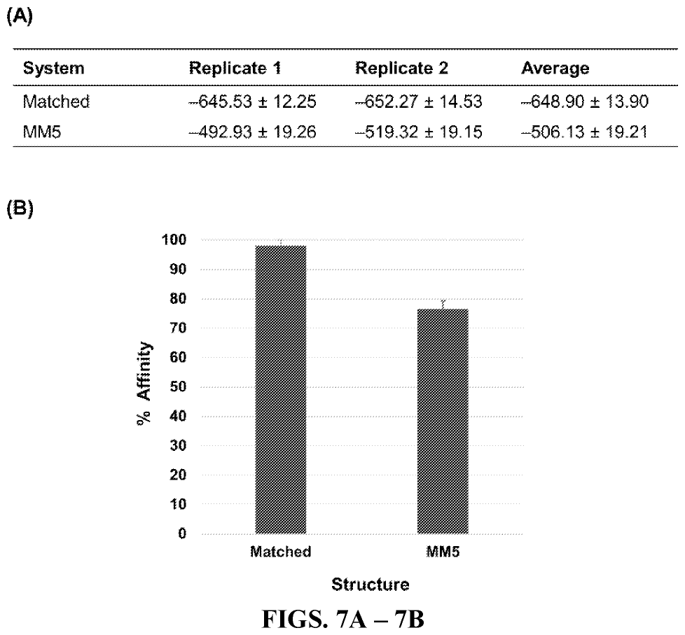

The PAM proximal mismatch (MM5) instigates local and allosteric conformational changes in the CRISPR-Cas9 system. The overall conformation of the Cas9·sgRNA.DNA tertiary complex remains stable with a mismatch at the fifth position from PAM in the MM5 system (FIG. 6). However, this mismatch in the DNA substrate induces several local and allosteric structural changes in the Cas9 and the nucleotides attached to it. For instance, as shown in FIG. 6B, interactions between the adjacent nucleotides and the mispair on the R loop (among t-DNA and sgRNA) are affected. Especially, the PAM distal end of the nt-DNA displays higher flexibility and losses interactions with the 3′-end of the t-DNA (FIGS. 6C and 6D, and FIG. 5B), indicating an allosterically modulated structural deviation in MM5. These differences partially provide an explanation for the calculated binding affinity reduction of ≈22% for the MM5 compared to the matched when considering the complexation of DNA with the Cas9-sgRNA binary complex (FIG. 7). Also observed were large amplitude motions of the protein domains directly involved with the nucleic acids in MM5: the recognition region (REC-I) interacting with the stem of sg-RNA and the C-terminal domain that binds the DNA.

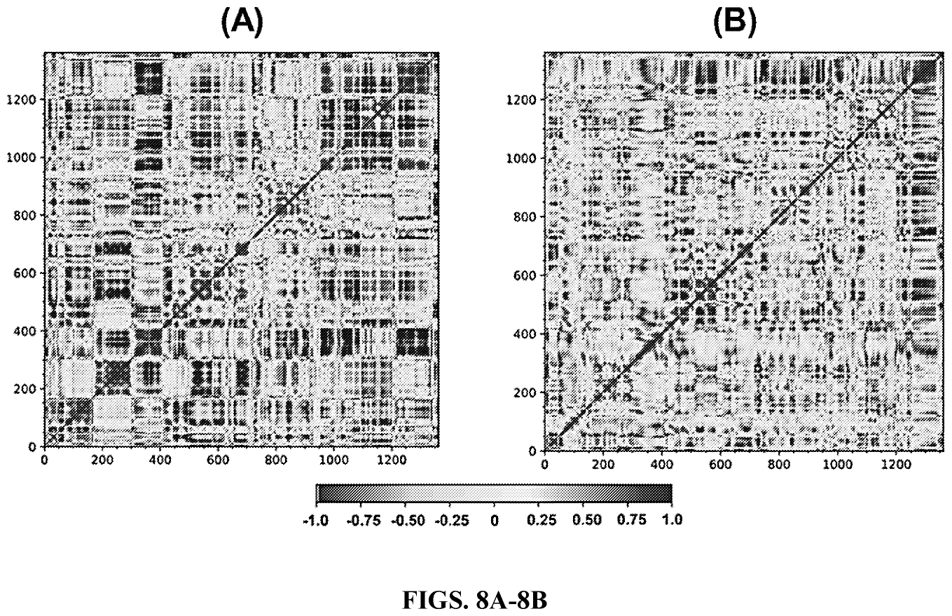

A dynamic cross-correlation analysis has been performed to characterize the large-scale motions of the Cas9 protein domains for matched and MM5, respectively (FIG. 8). Several deviations of the correlated motions of Cas9 domains are observed in MM5 upon the incorporated mismatch. The REC-II (167-307) and a part of REC-III (450-500) domain's movement along the direction of the HNH and RuvC-III domains (765-1099) in matched change into the opposite direction in MM5. On the other hand, the REC-I (94-167 and 307-447) region's anti-correlated motion in matched exhibits somewhat correlated motion in MM5 with these two nuclease domains. The HNH and RuvC-III regions show a positively correlated motion with a part of the CTD domain (1200-1368) in matched, while it is changed to a negatively correlated motion in MM5. Conversely, two regions of the REC-III (300-400 and 600-700) domain display an increased paired motion with the same CTD region in MM5, indicating a relative opening of the protein in MM5, which could affect the nucleotides and protein binding. Thus, the mismatch affects the overall motion of the Cas9.

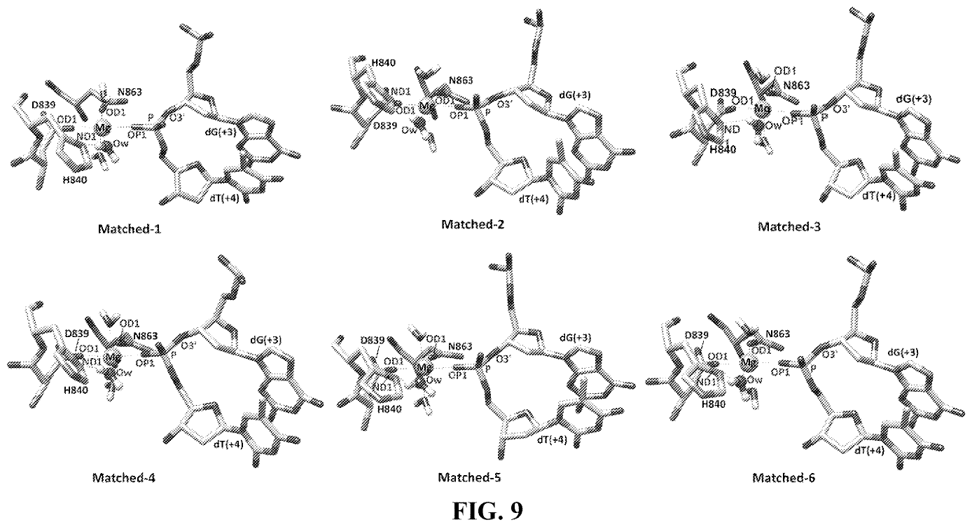

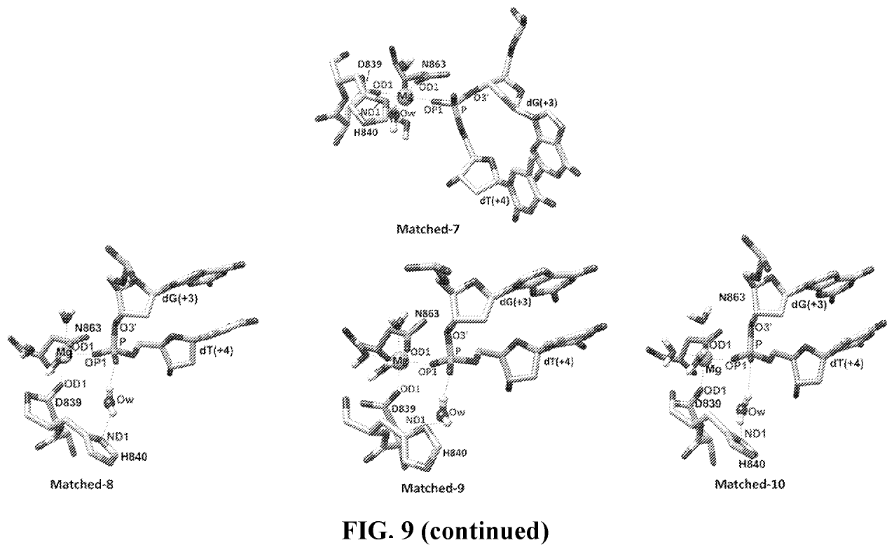

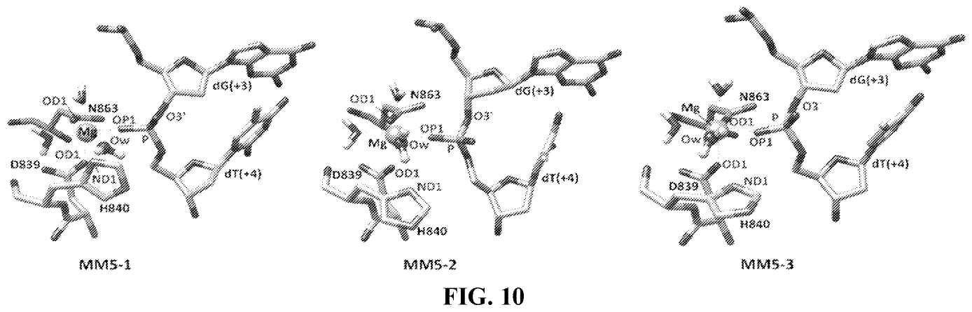

The mismatch weakens the cleavage point at the HNH catalytic site conformations. Based on the k-means analysis of ten clusters (thirty representatives) for each system, four clusters for matched and one cluster for the MM5 systems were selected by considering the most conducive orientations for the cleavage reaction in the active site (Tables 1 and 2, and FIGS. 9 and 10). In the case of the matched system, when one of the coordinated waters to the Mg²⁺ (termed first-shell water) acts as the nucleophile, the orientations of the active site are relatively suitable in three clusters, including ≈60% of the 100,000 simulated trajectories. Seven representatives of these three clusters, in which the mentioned water is also hydrogen-bonded to H840 (Matched-1 to Matched-7 in FIG. 9), were used for further QM/MM calculations. In comparison, when non-coordinated water around the Mg⁺² (termed second-shell water) is in a reasonable distance and orientation toward the H840 and the phosphate group, three representatives of the fourth cluster with a population abundance of 16.7% were considered for the further QM/MM studies (Matched-8 to Matched-10 in FIG. 9).

Contrary to the observed trend for the matched, about 13% of the clustered structures for the MM5 show a rotation of H840 that hinders its catalytic competence as the generalized base to activate the nucleophile. In addition, among the remaining 87%, only 15.9% (cluster 1) maintained catalytically conduce orientations, while even among the three representatives of this cluster, just one structure displays a good O3′-P . . . . O_{w} angle (Table 2 and FIG. 10). Furthermore, based on the clustering results in Table 2, the first-shell water was the only potential nucleophile in the MM5 structure. All the other representatives with the second-shell water are either too far from the H840 and phosphorus or the O3′-P . . . O_{w} angle is not suitable for the S_{N}2-like reaction. By considering the clustering result in total, more than 72% of the simulated trajectories of the matched favor the HNH active site conformation, leading to the catalytic cleavage of the t-DNA between the third and fourth nucleotides from the PAM region. In comparison, in ≈5% of the MM5 simulated trajectories, the orientations of the residues of the HNH's active site can lead to the cleavage reaction. This indicates a reduction of the precise and efficient cleavage of the t-DNA by mismatch containing MM5 compared to its native matched form.

TABLE 1
Important angles and distances between the residues of the Matched's
active site in all the representatives of the k-means clustering analysis.

| Cluster | Rep. | First-shell water | | | |
|---|---|---|---|---|---|
| No. | ID | MG [symbol] OP1[dT(+4)] | MG [symbol] OD1(D839) | MG [symbol] OD1(N863) | MG [symbol] ND1(H840) |
| 0 | 18016 | 1.99 | 1.98 | 2.10 | 4.59 |
| | 18481 | 1.98 | 1.97 | 2.08 | 4.62 |
| | 30863 | 1.96 | 1.98 | 2.09 | 4.55 |
| 1 | [symbol] | 1.98 | 2.02 | 2.06 | 4.57 |
| | 11126 | 1.95 | 1.95 | 2.03 | 4.61 |
| | 4168 | 2.04 | 1.98 | 2.07 | 4.58 |
| 2 | 38260 | 1.98 | 1.98 | 2.08 | 4.40 |
| | [symbol] | 1.99 | 1.98 | 2.08 | 4.45 |
| | 23730 | 1.99 | 1.98 | 2.09 | 4.46 |
| 3 | 78721 | 2.00 | 1.99 | 2.08 | 6.82 |
| | 95996 | 1.99 | 1.98 | 2.08 | 6.83 |
| | 96154 | 1.99 | 1.99 | 2.08 | 6.83 |
| 4 | 3542 | 1.99 | 1.98 | 2.08 | 4.75 |
| | 9419 | 1.98 | 1.97 | 2.07 | 4.74 |
| | 17533 | 2.00 | 1.98 | 2.07 | 4.76 |
| 5 | 61177 | 1.99 | 1.95 | 2.07 | 5.76 |
| | 57548 | 2.00 | 1.92 | 2.09 | 5.74 |
| | [symbol] | 2.00 | 1.98 | 2.06 | 5.77 |
| 6 | 57813 | 2.00 | 1.93 | 2.07 | 6.21 |
| | 61807 | 1.99 | 2.00 | 2.09 | 6.20 |
| | 54886 | 1.98 | 1.97 | 2.10 | 6.23 |
| 7 | 61847 | 2.00 | 1.99 | 2.05 | [symbol] |
| | 61747 | 2.00 | 1.92 | 2.07 | 5.31 |
| | 57269 | 2.02 | 2.05 | 2.06 | 5.29 |
| 8 | 57132 | 2.00 | 1.98 | 2.10 | 4.93 |
| | 57945 | 1.99 | 1.98 | 2.05 | 4.86 |
| | 61299 | 2.05 | 2.05 | 2.09 | 4.89 |
| 9 | 54774 | 1.95 | 1.98 | 2.07 | 6.72 |
| | 57452 | 1.98 | 1.96 | 2.05 | 6.69 |

| | |
|---|---|
| 54829 | no nucleophile |

| Cluster | Rep. | First-shell water | | | Second-shell water | |
|---|---|---|---|---|---|---|
| No. | ID | P [symbol] ND1(H840) | P [symbol] O [symbol] | O [symbol] P—O3′ | P [symbol] O [symbol] | O [symbol] P—O3′ |
| 0 | 18016 | 5.94 | 3.74 | 156.2 | N/A | N/A |
| | 18481 | 6.45 | 3.67 | 156.3 | N/A | N/A |
| | 30863 | [symbol] | 3.44 | 156.2 | N/A | N/A |
| 1 | [symbol] | 6.16 | 3.92 | 150.9 | N/A | N/A |
| | 11126 | 5.82 | 3.83 | 150.8 | N/A | N/A |
| | 4168 | 5.21 | 3.67 | 151.0 | N/A | N/A |
| 2 | 38260 | 5.16 | 3.90 | 149.0 | N/A | N/A |
| | [symbol] | 6.02 | 3.90 | 140.0 | N/A | N/A |
| | 23730 | 6.44 | 4.12 | 134.0 | N/A | N/A |
| 3 | 78721 | 6.19 | N/A | N/A | 3.36 | 164 5 |
| | 95996 | 5.86 | N/A | N/A | 3.41 | 154.7 |
| | 96154 | 6.16 | N/A | N/A | 3.63 | 163.4 |
| 4 | 3542 | 6.63 | 3.99 | 135.0 | N/A | N/A |
| | 9419 | 5.95 | 3.92 | 141.0 | N/A | N/A |
| | 17533 | 6.17 | 3.96 | 135.0 | N/A | N/A |
| 5 | 61177 | 6.26 | 3.38 | 108.8 | 4.56 | 149.4 |
| | 57548 | 6.03 | 3.78 | 102.7 | 3.76 | 156.4 |
| | [symbol] | 6.00 | 3.67 | 95.5 | 3.45 | 152.6 |
| 6 | 57813 | 6.52 | 3.44 | 100.1 | 3.76 | 148.3 |
| | 61807 | 6.79 | 3.65 | 106.0 | 3.72 | 147.0 |
| | 54886 | 6.26 | 3.62 | [symbol] | 3.56 | 145.9 |
| 7 | [symbol] | 5.81 | 3.39 | 105.0 | 3.62 | 153.4 |
| | [symbol] | 5.87 | 3.69 | 101.1 | 3.72 | 145.1 |
| | 57269 | 5.82 | 3.51 | 103.7 | 3.56 | 151.2 |
| 8 | 57132 | 5.67 | 3.64 | 101.9 | 3.66 | 146.5 |
| | 57945 | 5.60 | 3.63 | 119.2 | 3.97 | 156.9 |
| | 61299 | 5.34 | 3.66 | 112.2 | 3.49 | 159.3 |
| 9 | 54774 | 6.86 | 3.54 | 96.2 | 3.79 | 154.4 |
| | 57452 | 6.36 | 3.68 | 84.4 | 3.77 | 136.8 |

| | |
|---|---|
| 54829 | no nucleophile |

[symbol]

indicates data missing or illegible when filed

TABLE 2
Important angles and distances between the residues of the MM5's active
site in all the representatives of the k-means clustering analysis.

| Cluster | Rep. | First-shell water | | | |
|---|---|---|---|---|---|
| No. | ID | MG [symbol] OP1[dT(+4)] | MG [symbol] OD1(D839) | MG [symbol] OD1(N863) | MG [symbol] ND1(H840) |
| 0 | 61121 | 1.99 | 1.97 | 2.07 | 4.59 |
| | 66438 [symbol] | 1.98 | 1.97 | 2.08 | 4.60 |
| | 56372 | 1.99 | 1.97 | 2.06 | 4.60 |
| 1 | 8258 | 1.98 | 1.97 | 2.08 | 6.13 |
| | 12278 | 1.98 | 1.97 | 2.07 | 6.12 |
| | 43803 | 1.99 | 1.97 | 2.08 | 6.12 |
| 2 | 35577 | 1.98 | 1.97 | 2.07 | 4.99 |
| | 34846 | 1.99 | 1.98 | 2.07 | 5.00 |
| | 59699 | 1.99 | 1.97 | 2.07 | 5.01 |
| 3 | 7916 | 1.98 | 1.97 | 2.07 | [symbol] |
| | 11724 [symbol] | 1.98 | 1.97 | 2.06 | [symbol] |
| | 47386 | 1.98 | 1.97 | 2.07 | [symbol] |
| 4 | [symbol] | 1.99 | 1.97 | 2.06 | [symbol] |
| | 43719 | 1.98 | 1.96 | 2.06 | [symbol] |
| | [symbol] | 1.98 | 1.95 | 2.08 | [symbol] |
| 5 | 84171 [symbol] | 1.99 | 1.97 | 2.08 | 7.38 |
| | 89887% | 1.98 | 1.99 | 2.08 | 7.37 |
| | [symbol] | 1.97 | 1.97 | 2.09 | 7.38 |
| 6 | 88549 [symbol] | 2.00 | 1.98 | 2.08 | 7.95 |
| | 87256 | 1.98 | 1.98 | 2.07 | 7.94 |
| | 86414 [symbol] | 1.98 | 1.98 | 2.07 | 7.93 |
| 7 | 71241 | [symbol] | 1.98 | 2.15 | [symbol] |
| | 71237 | [symbol] | 1.98 | 2.06 | 6.59 |
| | 71236 [symbol] | 2.02 | 1.98 | 2.01 | 6.82 |
| 8 | 71192 | 1.96 | 1.97 | 2.12 | 6.12 |
| | 71191 | 1.93 | 1.97 | 2.12 | 6.14 |
| | 71196 | 1.95 | 1.97 | 2.02 | 6.04 |
| 9 | 71250 | 1.93 | 1.97 | 2.13 | 5.37 |
| | 71231 | 2.05 | 1.97 | 2.14 | 5.19 |
| | 71188 [symbol] | 1.95 | 1.97 | 2.16 | 5.13 |

| Cluster | Rep. | First-shell water | | | Second shell water | |
|---|---|---|---|---|---|---|
| No. | ID | P [symbol] ND1(H840) | P [symbol] O [symbol] | O [symbol] P—O3′ | P [symbol] O [symbol] | O [symbol] P—O3′ |
| 0 | 61121 | 5.40 | 3.85 | 122.7 | 3.76 | 125.0 |
| | 66438 [symbol] | 5.20 | 3.97 | 130.7 | N/A | N/A |
| | 56372 | 5.46 | 3.56 | [symbol] | 3.82 | [symbol] |
| 1 | 8258 | 6.70 | 3.86 | [symbol] | 4.15 | [symbol] |
| | 12278 | 6.43 | 3.44 | 132.0 | 4.25 | [symbol] |
| | 43803 | [symbol] | 4.14 | [symbol] | [symbol] | 124.6 |
| 2 | 35577 | 5.50 | 4.09 | 128.8 | 3.38 | 141.1 |
| | 34846 | [symbol] | 3.98 | 128.8 | 3.88 | 145.1 |
| | 59699 | 6.26 | 3.47 | [symbol] | 3.43 | 152.7 |
| 3 | 7916 | 5.75 | 4.07 | 127.1 | 3.87 | 143.6 |
| | 11724 [symbol] | 5.71 | 4.11 | 120.1 | N/A | N/A |
| | 47386 | 6.12 | 4.12 | 136.3 | [symbol] | 161.6 |
| 4 | [symbol] | 4.85 | 4.03 | 116.5 | 3.56 | 130.2 |
| | 43719 | 6.33 | 4.02 | 113.5 | 3.46 | 121.0 |
| | [symbol] | 6.25 | [symbol] | 134.7 | N/A | N/A |
| 5 | 84171 [symbol] | 7.73 | 4.01 | 114.3 | N/A | N/A |
| | 89887% | [symbol] | [symbol] | 105.9 | N/A | N/A |
| | [symbol] | [symbol] | 4.05 | 136.6 | 3.61 | 162.4 |
| 6 | 88549 [symbol] | 7.65 | 3.98 | 111.1 | N/A | N/A |
| | 87256 | 7.33 | 3.99 | 138.2 | 4.73 | 145.3 |
| | 86414 [symbol] | 7.36 | [symbol] | 123.1 | N/A | N/A |
| 7 | 71241 | 6.79 | [symbol] | 133.3 | 4.12 | 165.2 |
| | 71237 | 6.57 | 3.95 | 131.7 | 3.63 | [symbol] |
| | 71236 [symbol] | 7.02 | 3.82 | 127.5 | N/A | N/A |
| 8 | 71192 | 6.43 | 3.97 | 127.1 | 3.55 | 154.8 |
| | 71191 | 6.22 | [symbol] | 131.9 | 3.71 | 164.1 |
| | 71196 | 6.27 | 3.85 | 120.3 | 3.53 | 169.0 |
| 9 | 71250 | 5.54 | [symbol] | 122.4 | 3.68 | [symbol] |
| | 71231 | 5.49 | [symbol] | 141.6 | 3.92 | [symbol] |
| | 71188 [symbol] | [symbol] | 3.98 | 134.6 | N/A | N/A |

[symbol]

Representatives in which second-shell water between the H840 and phosphodiester bridge does not exist. [symbol]
Representatives in which the H840 is flipped and not facing the active site. [symbol]
indicates data missing or illegible when filed

Conformation of the reactants for matched and MM5 systems. Based on the clustering analysis results, ten representatives of the matched shown in FIG. 9 were selected for the further hybrid QM/MM studies. Since representatives with either the first- or the second-shell water were chosen via the clustering; thus, two sets of structures were considered separately to be optimized for further calculation of the relative optimization energies.

TABLE 3
The results of the k-means clustering analysis and the
calculated relative QM/MM optimization energies for the
selected representatives of the matched and the MM5.

| | k-means clustering¹ | | | | Rel. opt. |
|---|---|---|---|---|---|
| | Cluster | No. of | Pct. | | ener [symbol] |
| Structure | No. | snapshots | (%) | Rep. ID | (eV)² |
| Matched^{1st shell} | 1 | 29701 | 29.7 | Matched-1 | 16.2 |
| | | | | Matched-2 | 21.4 |
| | | | | Matched-3 | 7.8 |
| | 2 | 23454 | 23.5 | Matched-4* | 0.0 |
| | | | | Matched-5 | 3.0 |
| | | | | Matched-6 | 11.0 |
| | 3 | 7227 | 7.2 | Matched-7 | 18.2 |
| Matched^{2nd shell} | 4 | 16698 | 16.7 | Matched-8* | 0.0 |
| | | | | Matched-9 | 3.8 |
| | | | | Matched-10 | 19.4 |
| MM5³ | 1 | 15872 | 15.9 | MM5-1* | — |

¹Values of the k-means clustering analysis are as follows: cluster numbers, the number of snapshots in each cluster, the percentage of the population abundance in each cluster, and the representative ID.
²QM/MM optimization energies were calculated at ωB97X-D/6-31G** level of theory with AMBER ff14SB Force Field.
³No relative optimization energy is provided for the MM5 since only one structure was used for the optimizati [symbol]
*Matched-4, Matched-8, and MM5-1 are used for further calculations and will be called Matched^{1st shell}, Matched^{2nd shell}, and MM5, respectively. [symbol]
indicates data missing or illegible when filed

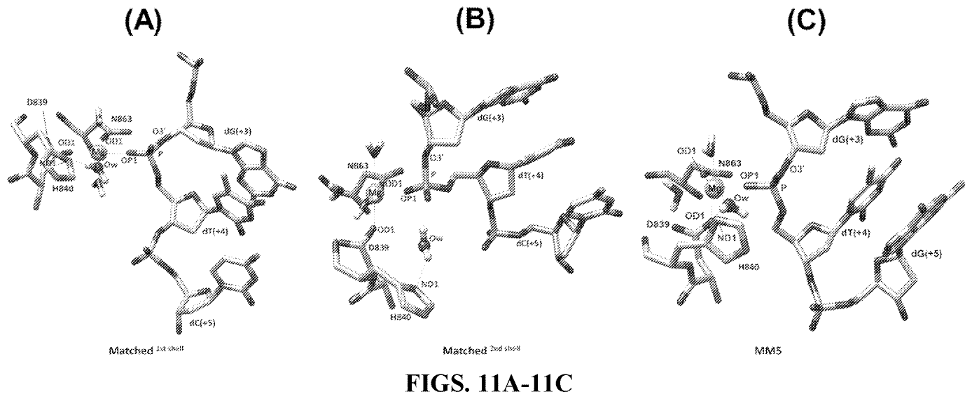

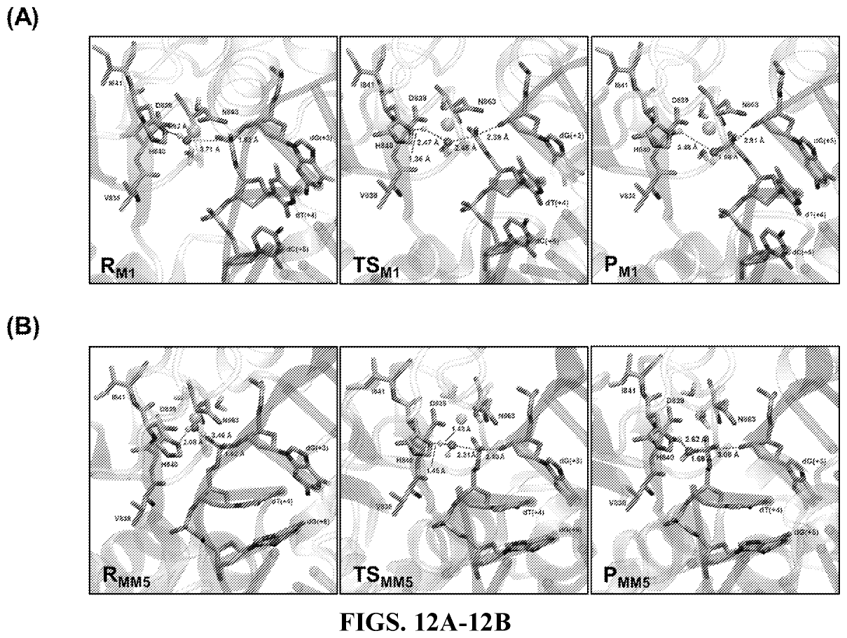

A summary of the k-means clustering for the selected representatives and the calculated relative optimization energies are listed in Table 3. The table shows that Matched-4 and Matched-8 are the most stable structures of the first- and the second-shell water reactants (Matched^{1 st shell} and Matched^{2 nd shell}) respectively. Regarding the MM5, one representative of the clustering (MM5-1) had reasonable orientations of the active site; thus, this structure was optimized and used for designing the product (MM5). The optimized structures of the active site of the Matched^{1 st shell} Matched^{2 nd shell}, and MM5 are shown in FIG. 11.

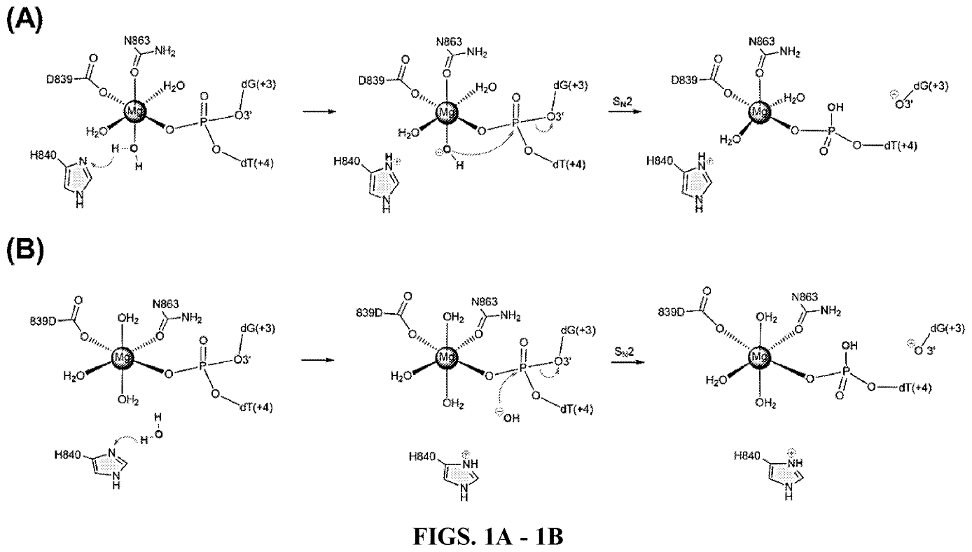

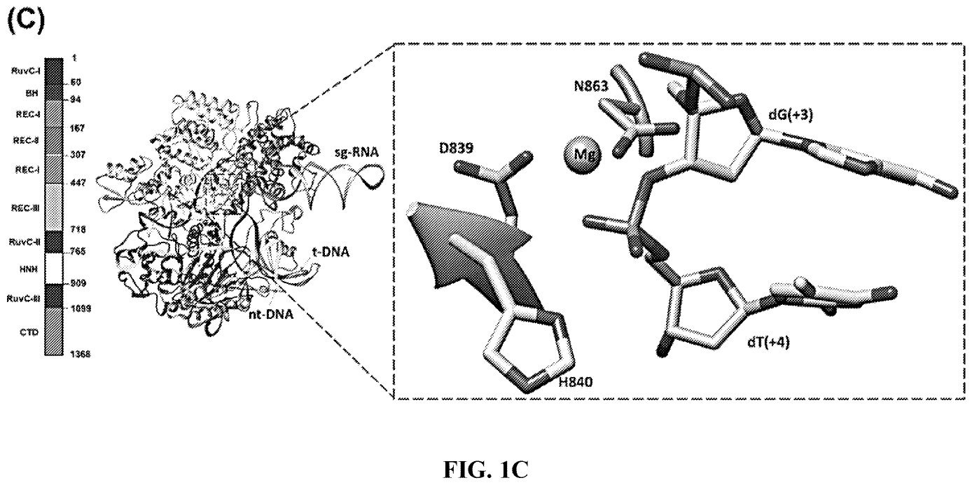

Based on the position of the nucleophilic water in the representatives, two pathways are considerable for the DNA cleavage mechanism at the HNH domain via an S_{N}2-like reaction. In the first pathway (FIG. 1A), predominantly seen for the Matched^{1 st shell} and the MM5, the first-shell water plays the nucleophile's role. A proton transferring occurs from the water to H840, and the resulting OH attacks the phosphorus with concomitant cleavage of the P—O3′ bond of the dG(+3). In the second pathway shown in FIG. 1B, the second-shell water between the phosphate bridge and the H840 undergoes the proton transfer and performs the cleavage reaction. Since both pathways involve a water molecule for the phosphodiester bond cleavage reaction, these follow hydrolysis mechanisms.

The t-DNA hydrolysis by the matched system. As mentioned above, the hydrolysis of t-DNA by the HNH domain of endonuclease Cas9 in the matched system can occur through one of the following pathways: (1) metal-bound water/first-shell water-mediated pathway or (2) second-shell water-assisted pathway.

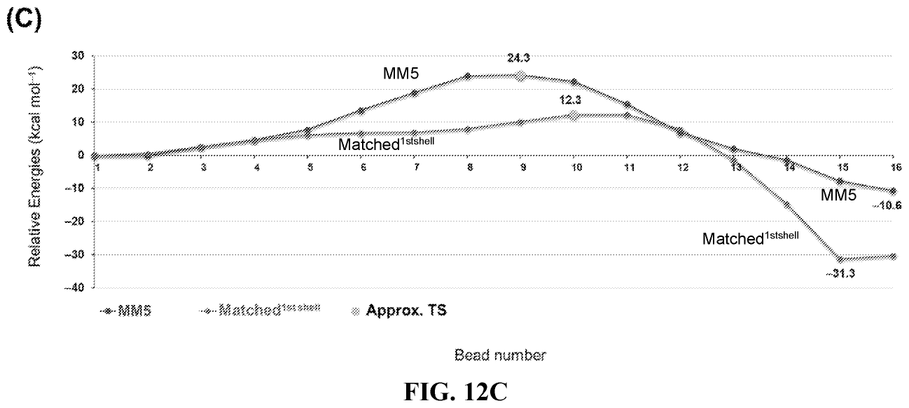

1. Metal-bound water/first-shell water-mediated pathway (M1 pathway). In the reactant of this pathway (R_{M1}, FIG. 12A), one phosphoryl oxygen (OP1) atom of dT(+4) is bound to the Mg²⁺ ion (Mg . . . OP1=2.04 Å), while the other one (OP2) interacts with the residue Q844 through a hydrogen bond. This metal-substrate (t-DNA) coordination activated the scissile P-O3′ bond of dG(+3) by 0.01 Å in comparison to the P—O5′ bond of dT(+4) (P-O3′=1.63 Å and P-05′=1.62 Å in Table 4). The positive charge of the magnesium ion (1.94 e) plays a vital role in activating the P—O3′ bond. Additionally, this coordination mode helps polarize the scissile phosphodiester bond's P atom (1.38 e). In R_{M1}, the base residue H840 is hydrogen bonded to an Mg-bound water molecule, HwOwH (Mg-Ow=2.07 Å and Hw-Nδ=1.91 Å in Table 4). The catalytic site residues D839, N863, and two additional water molecules complete the octahedral coordination geometry around the Mg²⁺ ion. In the approximate transition state (TSM1), H840 abstracts the Hw proton from the water (HwOwH), and the resulting OwH nucleophile attacks the electrophilic P atom (1.38 e) of the dT(+4) concomitantly, elongating the P—O3′ bond. This process occurs with a barrier of 12.3 kcal mol⁻¹, and all key distances demonstrate the concerted nature of this step (Hw-Nδ=1.36 Å, Ow-P=2.45 Å and P-O3′=2.39 Å in FIG. 12 and Table 4). In the final product (PMI), the P—O3′ phosphodiester bond cleavage is completed by separating the t-DNA into two parts. The formation of PMI is exergonic by 30.3 kcal mol⁻¹ from R_{M1}. As shown in FIG. 12A, the octahedral geometry around the Mg²⁺ ion changes to trigonal bipyramidal during the cleavage reaction (R_{M1} to P_{M1}), and the coordination number changes from six to five. Several experimental and computational studies have been reported on the catalytic mechanism of Cas9.54, 55, 115-122 Sue and coworkers employed several kinetic techniques and successfully characterized each major step of the CRISPR/Cas9 mechanism.123 They showed that the DNA cleavage (chemistry step) from a pre-formed ternary complex (Cas9·sgRNA·DNA) to form DNA products is fast (K_{chem}≥700 s⁻¹). Their results are in good agreement with other studies on the human AP endonuclease 1 (APE1), in which the obtained values were K_{chemistry}≥700 s⁻¹ and K_{cat}≥850 s^{-1. 124, 125} Based on the observed rate of the reaction by Sue and coworkers, the experimental estimated free energy barrier (AG+) based on Eyring's TS theory for the cleavage reaction is ≤14.1 kcal mol 1. The calculated potential energy barrier of 12.3 kcal mol⁻¹ is around 2 kcal mol⁻¹ below the estimated experimental barrier.

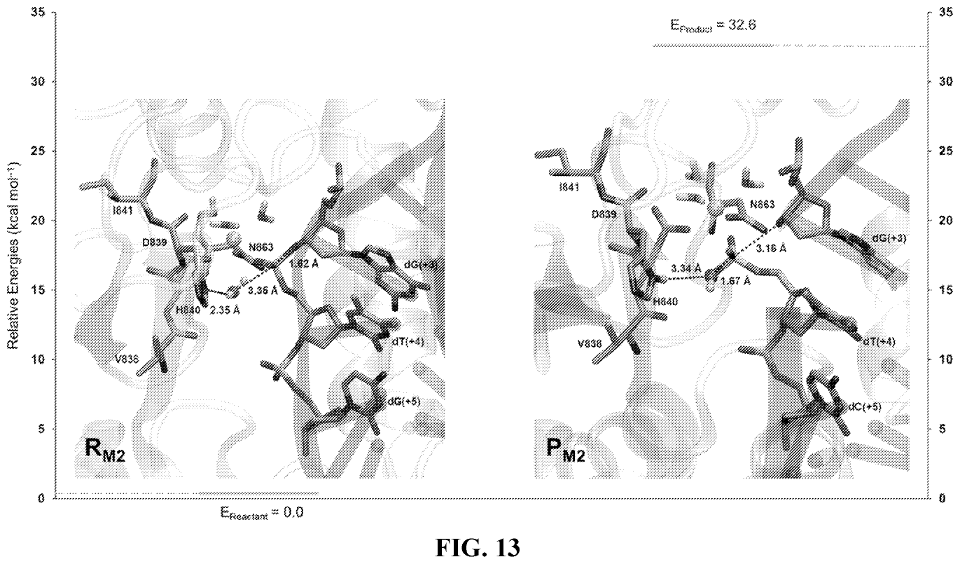

2. Second-shell water-assisted pathway (M2 pathway). The major difference in this pathway is that the base residue H840 creates a nucleophile by activating an external water molecule that is not bound to the Mg²⁺ ion (FIG. 1B). In the optimized reactant (R_{M2}, FIG. 13, and Table 4), the P—O3′ bond is 0.01 Å less activated than in R_{M1} due to the low Lewis acidity of the Mg²⁺ ion in this configuration. In addition, the Ow-Hw bond in the second-shell water molecule is 0.01 Å less activated than that of metal-bound water in the previous pathway, resulting in a relatively weak nucleophile (Table 4). Moreover, the charge of the P atom is reduced by 0.21 e compared to that of R_{M1}. Although the Ow . . . P—O3′ angle in R_{M2} (≈163°) is closer to the desired angle for S_{N}2-type reaction than that of R_{M1} (≈151°), there seems to be a competition among the H840 and the free phosphoryl oxygen (OP2) of the t-DNA substrate to abstract a proton from the nucleophile water (HwOwH). This is indicated by an additional strong hydrogen bond (1.80 Å) between the H atom of the nucleophile water and OP2 in R_{M2}. In addition, the orientation of this water molecule is not in a favorable position for the nucleophilic attack on the P atom of the substrate. The optimized product (P_{M2}) is endergonic by 32.6 kcal mol⁻¹ from R_{M2}, indicating the unfavorable nature of this mechanism. Thus, the calculations suggest that the second-shell water molecule is a weaker nucleophile than the metal-bound water for this reaction. This is also seen in previous studies related to phosphodiester bond hydrolysis reaction by single metal containing nucleases.^{126, 127}

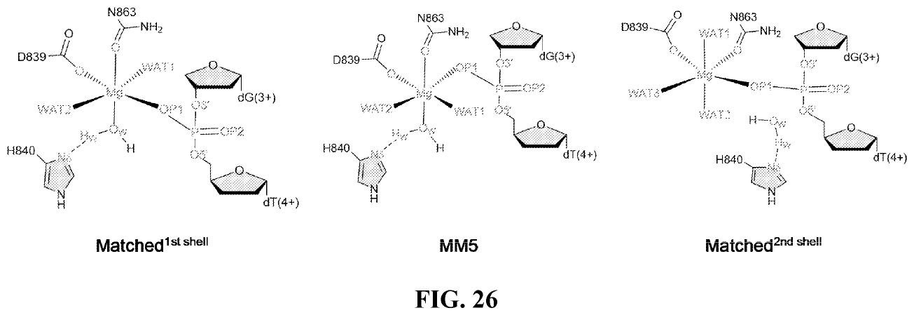

TABLE 4
Important bond distances and ESP charges of the critical structures (Reactant,
TS, and Product) during the cleavage reaction at the active site of the
Matched and MM5. The geometry of the reactant's active site is also
given in FIG. 26 for a better understanding of the atomic labels.

| | Matched^{1st shell} | | | MM5 | | | Matched^{2nd shell} | |
|---|---|---|---|---|---|---|---|---|
| | R_{M1} | TS_{M1} | P_{M1} | R_{MM5} | TS_{MM5} | P_{MM5} | R_{M2} | P_{M2} |
| Important Distances | | | | | | | | |
| P_{dT(+4)}—O3′_{dG(+3)} | 1.63 | 2.39 | 2.91 | 1.62 | 2.40 | 3.08 | 1.62 | 3.16 |
| P_{dT(+4)}—OP1_{dT(+4)} | 1.50 | 1.50 | 1.53 | 1.51 | 1.44 | 1.51 | 1.50 | 1.50 |
| P_{dT(+4)}—OP2_{dT(+4)} | 1.50 | 1.48 | 1.51 | 1.50 | 1.45 | 1.50 | 1.51 | 1.50 |
| P_{dT(+4)}—O5′_{dT(+4)} | 1.62 | 1.62 | 1.62 | 1.61 | 1.56 | 1.61 | 1.62 | 1.61 |
| O3′_{dG(+3)}—C3′_{dG(+3)} | 1.41 | 1.36 | 1.33 | 1.42 | 1.41 | 1.40 | 1.42 | 1.33 |
| MG-OP1_{dT(+4)} | 2.04 | 2.01 | 1.98 | 2.02 | 2.00 | 2.01 | 2.06 | 2.04 |
| MG-O_{D839} | 2.04 | 2.04 | 2.11 | 1.98 | 1.99 | 2.01 | 2.02 | 2.06 |
| MG-O_{N863} | 2.10 | 2.03 | 2.02 | 2.06 | 1.95 | 1.95 | 2.10 | 2.09 |
| MG-O_{WAT1} | 2.12 | 2.08 | 2.20 | 2.21 | 1.82 | 2.12 | 2.16 | 2.12 |
| MG-O_{WAT2} | 2.11 | 2.03 | 2.04 | 2.08 | 2.10 | 2.22 | 2.16 | 2.19 |
| MG-O_{WAT3} | N/A | N/A | N/A | N/A | N/A | N/A | 2.13 | 2.17 |
| MG-O_{W} | 2.07 | 2.60 | 3.00 | 2.17 | 2.82 | 3.70 | 4.47 | 4.08 |
| O_{W}-P_{dT(+4)} | 3.71 | 2.45 | 1.66 | 3.40 | 2.31 | 1.66 | 3.35 | 1.67 |
| O_{W}-H_{W} | 0.97 | 2.47 | 3.48 | 0.97 | 1.43 | 2.62 | 0.96 | 3.34 |
| H_{W}-Nδ_{H840} | 1.91 | 1.36 | 1.02 | 2.06 | 1.45 | 1.03 | 2.35 | 1.01 |
| ESP charges | | | | | | | | |
| MG | 1.94 | 1.55 | 1.49 | 1.84 | 1.46 | 1.31 | 1.43 | 1.41 |
| O3′_{dG(+3)} | −0.45 | −0.66 | −0.79 | −0.41 | −0.72 | −0.76 | −0.41 | −0.89 |
| P_{dT(+4)} | 1.38 | 1.25 | 1.09 | 0.76 | 0.77 | 0.81 | 1.17 | 1.17 |
| OP1_{dT(+4)} | −0.94 | −0.87 | −0.81 | −0.69 | −0.52 | −0.51 | −0.70 | −0.63 |
| OP2_{dT(+4)} | −0.86 | −0.79 | −0.77 | −0.73 | −0.68 | −0.73 | −0.82 | −0.82 |
| O5′_{dT(+4)} | −0.35 | −0.32 | −0.26 | −0.20 | −0.07 | −0.24 | −0.39 | −0.39 |
| O_{W} | −1.16 | −1.08 | −0.79 | −0.82 | −0.74 | −0.63 | −0.88 | −0.86 |
| H | 0.61 | 0.58 | 0.32 | 0.32 | 0.39 | 0.42 | 0.39 | 0.33 |
| H_{W} | 0.44 | 0.36 | 0.53 | 0.41 | 0.27 | 0.37 | 0.41 | 0.45 |
| ND_{H840} | −0.44 | −0.22 | −0.17 | −0.44 | −0.26 | −0.19 | 0.30 | −0.16 |

The t-DNA hydrolysis by MM5 system. As evidenced by the cluster analysis, only one of the extracted representative structures provides a suitable active site configuration for the t-DNA cleavage reaction by the HNH catalytic site of MM5. This structure possessed a water molecule bound to Mg²⁺ ion and hydrogen bonded to H840 that can be used as the potential nucleophile of the hydrolysis reaction. Thus, the M1 pathway for MM5 was investigated to understand the structural and mechanistic details involved to implement the findings to mitigate the knowledge gap between mismatch sensitivity and specificity of Cas9.

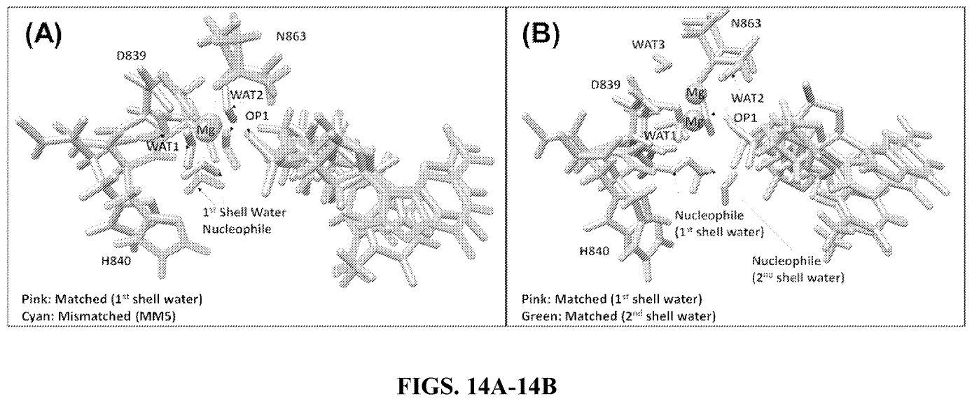

The optimized reactant of MM5 (R_{MM5} in FIG. 12B) is different from the optimized reactant of the matched^{1st shell} (R_{M1} in FIG. 12A) due to the position of a water molecule (WAT2) bound to the Mg²⁺ ion (see FIG. 14). A reduction of 0.10 e of the charge of the Mg²⁺ ion in R_{MM5} along with a decrease of 0.01 Å for the P—O3′ bond compared with the matched system (R_{M1}) are observed. The nucleophile OwH of R_{MM5} has a reduced charge of 0.03, and the P atom of the scissile phosphodiester bond shows a reduction of 0.6 e compared with R_{M1}. Moreover, the Ow . . . P—O3′ angle in the R_{MM5} is ≈141°, shorter than the expected 180° for an S_{N}2 attack. Additionally, the water's oxygen does not face the phosphorus in a catalytically conducive orientation, and the Hw-Ow . . . P angle is unfavorable (≈40°). This angle should be around 109° in the product, which results in the requirement of a significant rotation by the water in the reactant to reach a catalytically competent orientation. These differences help explain (at least in part) the higher activation barrier for the MM5 (TS_{MM5}=24.3 kcal mol⁻¹). In the approximated transition state, the breaking and forming bond distances (Ow-P=2.31 Å and P-O3′=2.40 Å, Table 4) display a concerted (S_{N}2-like) dissociative pathway¹²⁸ where a slightly more bond cleavage to the leaving group than bond formation to the nucleophile is observed (P—O3′ is 0.09 Å longer than Ow-P).

The matched system follows a concerted associative pathway¹²⁹⁻¹³¹ with a similar extent of partial bond formation to the nucleophile and partial bond cleavage to the leaving group at the transition state, TS_{M1} (Ow-P=2.45 Å and P-O3′=2.39 Å, Table 4). Unlike the matched system (M1 path), Mg²⁺ loosely binds to the water nucleophile (Mg-Ow=2.17 Å, in RMM5), indicating that it does not act as a suitable Lewis acid in MM5. The tightness of the transition state for matched and MM5, described in terms of the Ow-P (nucleophile) and P—O3′ (leaving group) bond distances, decreases from matched to MM5 (4.84 Å vs. 4.71 Å). The sum of the OW-P and P—O3′ distances illustrates the hydrolytic reaction's progression. Comparing these distances for the matched system in R_{M1} and TSM1 indicates a significant increase (0.50 Å) in tightness from 5.34 Å to 4.84 Å. Conversely, the increment of tightness is only 0.31 Å in the MM5 case (R_{MM5} and TSMM5), indicating a relatively low reaction progression, which is also aligned with the obtained almost doubled (12 kcal mol⁻¹ higher) activation barrier for the MM5 compared to the matched. Moreover, in a recent paper,¹³² kinetic rates of the DNA cleavage reaction for a similar system have been calculated using a kinetic model designed for plasmid DNA cleavages. The relative cleavage rates for matched DNA and MM5 DNA were 1.23±0.13 min⁻¹ and 0.68±0.09 min-1, respectively. Since these rates involve the whole kinetic process up to the cleavage step, a direct comparison with the values is not possible. However, if the relative rates are considered, matched DNA shows almost twice faster reaction, similarly to what was see from the barriers.

Based on the reactant and product's QM/MM optimization energies, the cleavage reaction catalyzed by the Matched^{1 st shell} is exoergic with the reaction energy of −30.3 kcal mol⁻¹, while this value is −10.6 and 32.6 kcal mol⁻¹ for MM5 and Matched^{2 nd shell}, respectively. The energy differences between the reactant and product of the Matched^{2 nd shell} and MM5 compared to the Matched^{1st shell} might be related to the intermolecular interactions between the attacking water and its surrounding residues. Hence, the noncovalent interactions (NCI) of the nucleophilic waters in the reactant of each system were analyzed and visually compared by the promolecular density method.

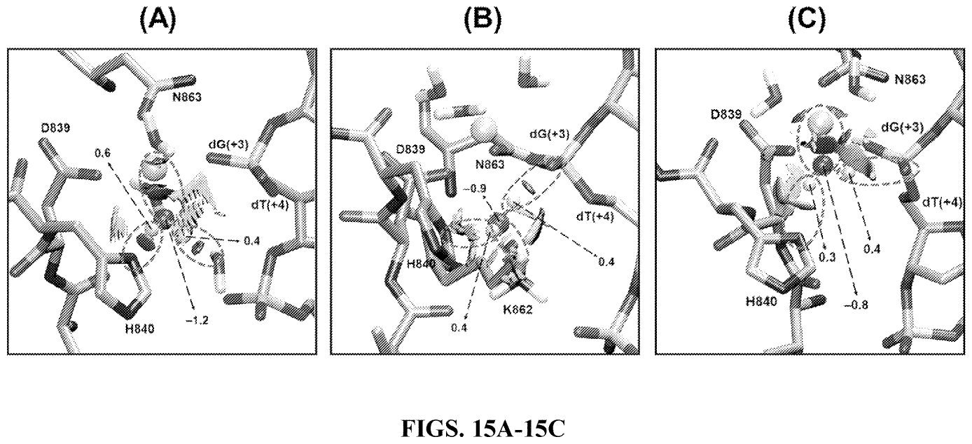

FIGS. 15A and 15C show that the nucleophilic waters in the Matched^{1 st shell} and MM5 have strong, attractive interactions with Mg²⁺ (in the NCI scale) and perform hydrogen bonds with H840, but in the latter one, the color of the surfaces between the nucleophilic water and H840 show weaker Hw . . . N8 hydrogen bond than the former one. It also can be seen that the other hydrogen of the nucleophilic water in the Matched^{1 st shell} has a strong hydrogen bond with adjacent water, which stabilizes the nucleophilic attack. In contrast, the second hydrogen of the nucleophilic water in MM5 has weak hydrogen bond interaction with the OP1 of dT(+4), which does not favor the nucleophilic attack. The NCI plot for the Matched^{2 nd shell} in FIG. 15B explicitly shows that the nucleophilic water creates a hydrogen bond with the ζ-hydrogen of K862, and the OP1 of dT(+4), while displaying a weak van der Waals interactions with H840, which is not suitable for the proton transfer step. The calculated ESP charges in Table 4 also show that the nucleophilic water in the Matched^{1 st shell} is more polarized than the nucleophilic water in the Matched^{2 nd shell} and MM5, facilitating the proton transfer from the water to H840 in the Matched^{1 st shell}.

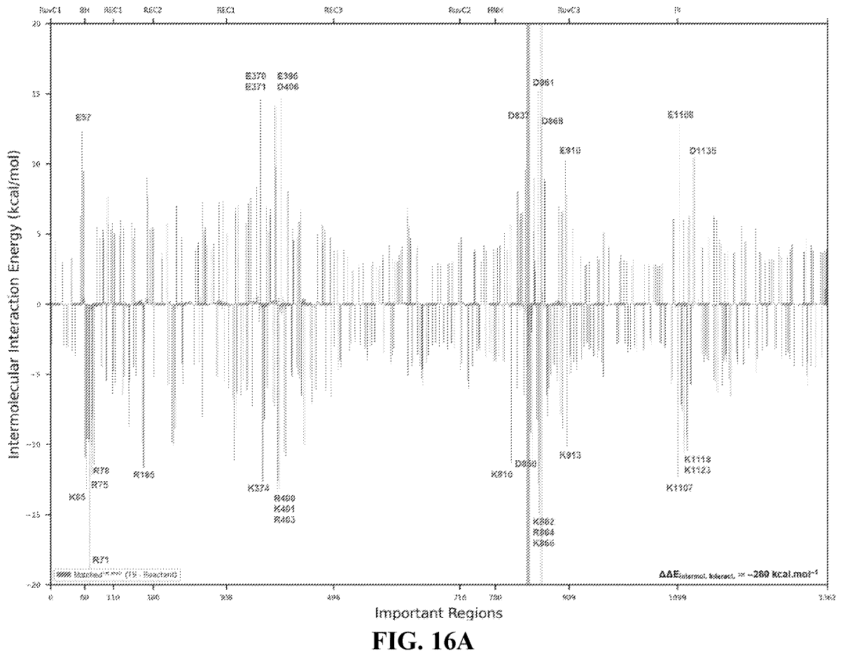

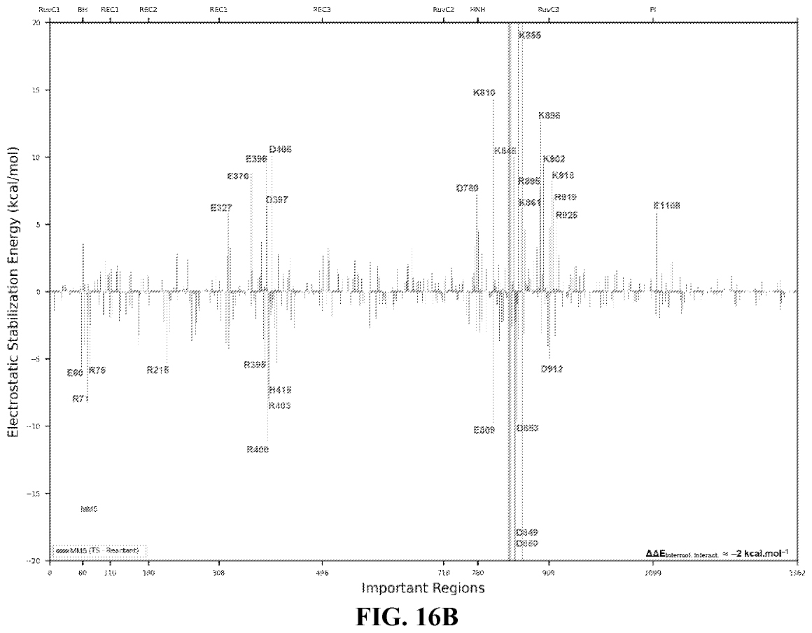

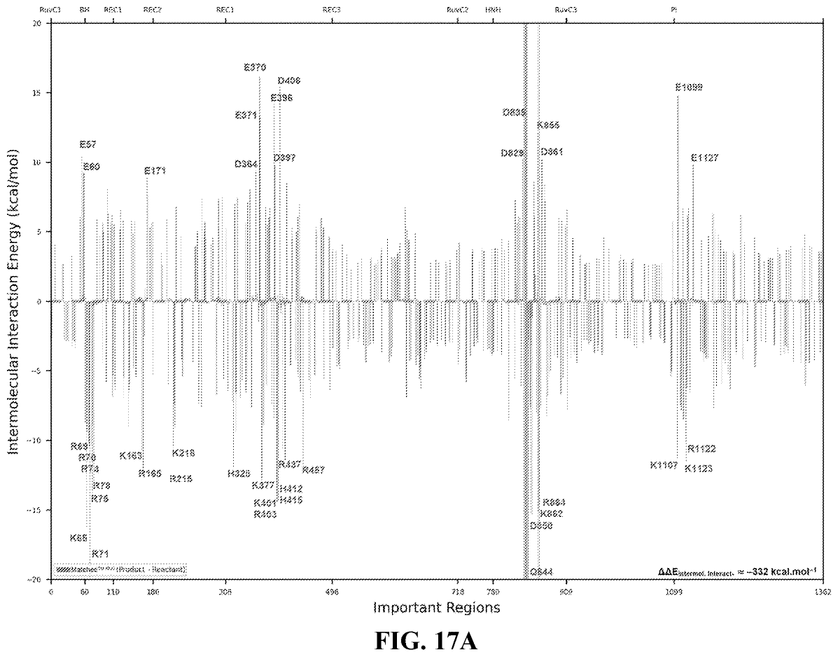

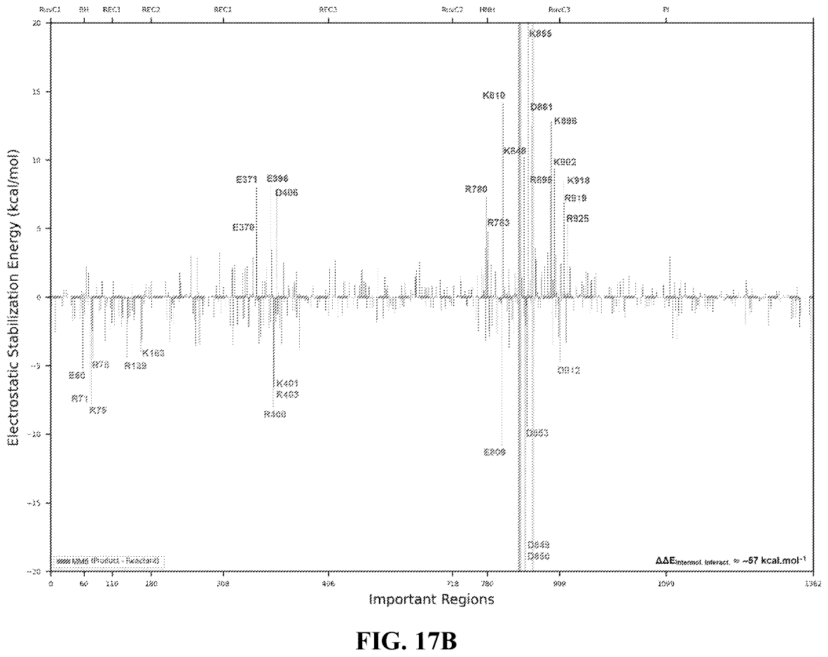

Energy Decomposition Analysis (EDA) reveals candidate Cas9 residues that could possibly reduce off-target cleavages. EDA was performed on the reactant, TS, and product structures of the matched and MM5 to study the non-bonded intermolecular interactions (Coulomb and van der Waals) between the Cas9 and the residues of the active site. Calculated intermolecular interaction energy differences, ΔΔE_{Intermol.Interact}, for the protein and nucleic acid residue between the reactant and TS of the matched and MM5 are −280 and −2 kcal mol⁻¹, respectively (see Eqn. 1). These results show that the protein environment significantly stabilizes the transition state of the Matched^{1 st shell} system compared to the MM5. Corresponding values between the product and the reactant of the Matched^{1 st shell} and MM5 are −332 and −57 kcal mol⁻¹, respectively, which shows the protein environment also favors the stabilization of the product of the matched system compared with MM5. Detailed graphs showing the stabilizing and destabilizing residues along the reactant-to-TS (kinetics of the reaction) and reactant-to-product (thermodynamics of the reaction) steps are given in FIGS. 16 and 17, respectively.

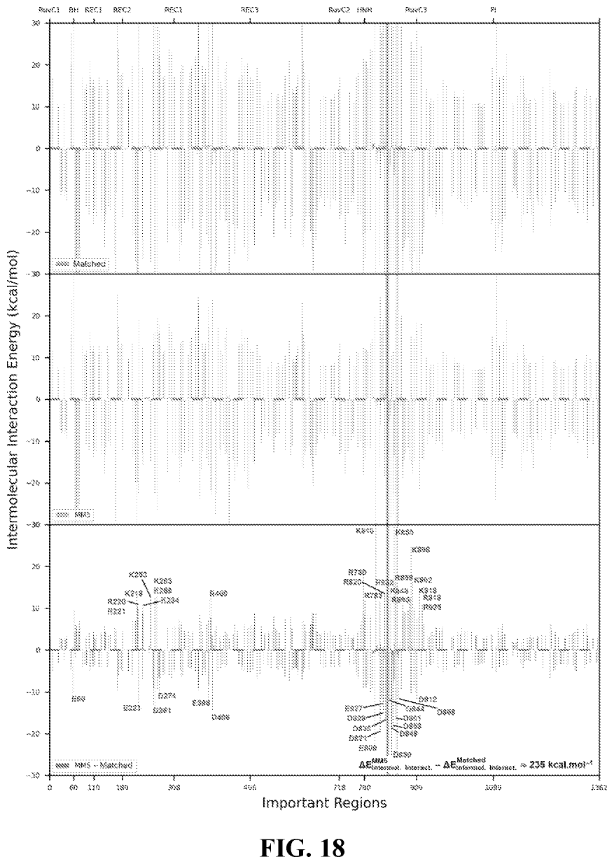

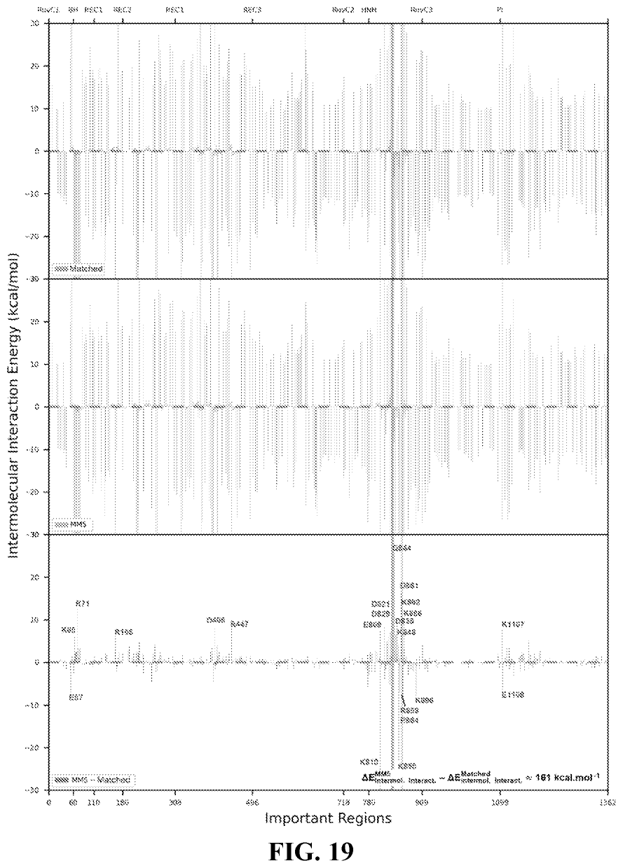

Understanding how stable the reactant and product of the matched are compared to the MM5 gives a good insight into the residues with considerable stabilizing and destabilizing effects on these structures. Calculated ΔΔE_{Intermol.Interact.} between the reactants or products of the MM5 and matched are 235 and 161 kcal mol⁻¹, respectively (detailed results in FIGS. 18 and 19). This shows that the MM5 gets less stable by the Cas9 than the matched, and this destabilizing effect is more significant in the reactant than the product. In fact, the Cas9 helps stabilize the product of the reaction during the catalytic reaction by the MM5, but this stabilization is much less than that in the matched. Decomposing the free enthalpy contributions to the binding enthalpies of the matched and MM5 on a per-residue basis was also performed to study the binding affinities between the active site and the rest of the system via the MM/GBSA approach. The calculations show that the binding affinities in the reactant of the matched are higher than the MM5. The average values of ΔHₜₒₜₐₗ are −161.15 and −142.65 kcal mol⁻¹ for the matched and MM5, respectively (detailed values in Table 5). Calculated binding enthalpies for the products show the same trend as the reactants and the values are −153.75 kcal mol⁻¹ for the matched and −140.32 kcal mol⁻¹ for the MM5.

TABLE 5
Calculated binding enthalpies between the HNH's active site¹ and the rest of the system² for the
Matched^{1st shell} and the MM5 via the MM/GBSA approach. All the calculated terms are in kcal mol⁻¹.

| | | | | | | | | | | ΔHₜₒₜₐₗ ± |
|---|---|---|---|---|---|---|---|---|---|---|
| System | Str. | Rep. | Bonded³ | VDW⁴ | E_{EL}⁵ | E_{GB} | E_{Surf} | ΔH_{gas} | ΔHₛₒₗᵥ | (Std. Dev.) |
| Matched | Reactant | 1 | 57.72 | −103.76 | 1920.27 | −2012.41 | −15.05 | 1874.22 | −2027.46 | −153.23 ± 7.19 |
| | | 2 | 57.14 | −112.41 | 1803.14 | −1900.88 | −16.07 | 1747.88 | −1916.95 | −169.07 ± 8.29 |
| | Product | | 53.74 | −103.27 | 1755.89 | −2034.51 | −15.41 | 1896.17 | −2049.92 | −153.75 ± 6.20 |
| MM5 | Reactant | 1 | 60.69 | −105.04 | 1824.23 | −1912.38 | −14.39 | 1779.88 | −1926.77 | −146.89 ± 7.79 |
| | | 2 | 58.10 | −105.91 | 1856.29 | −1932.19 | −14.70 | 1808.48 | −1946.89 | −138.41 ± 9.35 |
| | Product | | 65.31 | −100.02 | 1888.93 | −1980.41 | −14.13 | 1854.22 | −1994.54 | −140.32 ± 7.01 |

¹HNH's active site is considered as the ligand and the corresponding residues are 838-841, 863, 1493-1495, and 1541.
²Rest of the system is considered as the receptor and the corresponding residues are 1-837, 842-862, 864-1492, 1496-1540, and 1542-1543.
³Bonded term is made up of Bond + Angle + Dihedral terms.
⁴VDW is consist of van der Waals + 1-4 van der Waals terms.
⁵E_{EL} is made up of E_{Electrostatic} + E_{1-4 Electrostatic} terms.

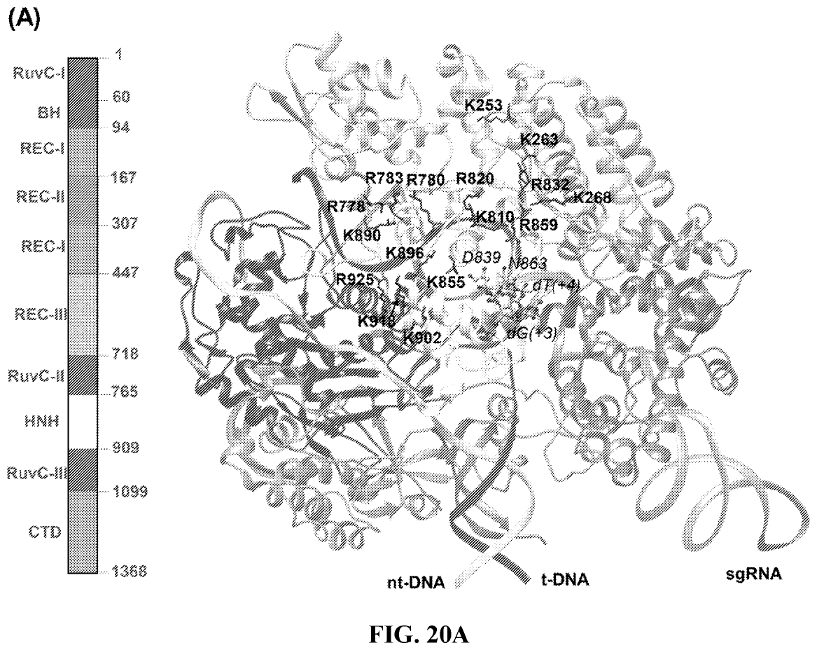

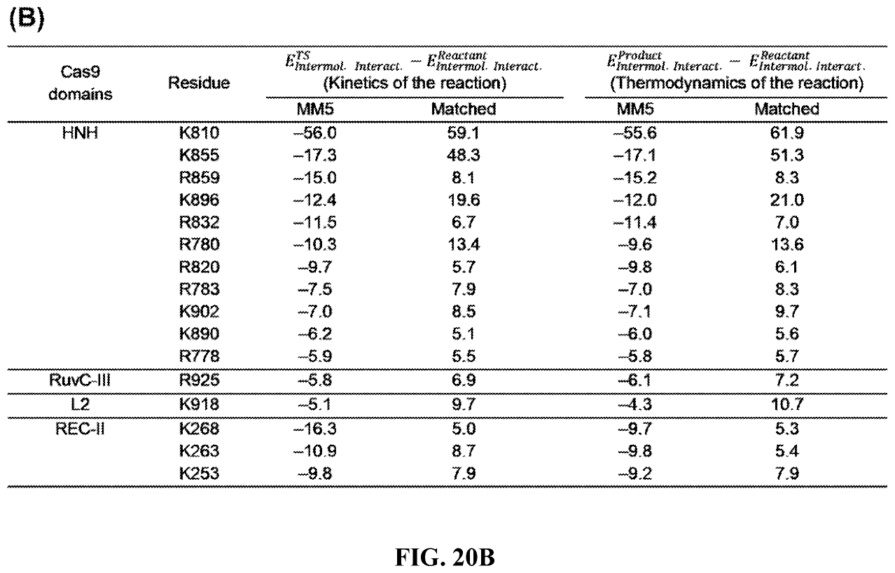

Several residues that show differential effects in the matched and MM5 systems were identified (FIG. 20 and Table 6), which have been previously recognized.133-135 For instance, the high-fidelity Cas9 variants (SpCas9-HF1 to SpCas9-HF4) identified by Joung and coworkers¹³⁶ contain R661 residue mutation (R661 to A), which is also one of the candidate residues (Table 6). Slaymaker et al.¹³³ employed a structure-guided engineering approach on SpCas9 to improve its DNA targeting specificity. Three high fidelity variants of SpCas9 (K855A), (K810A/K1003A/R1060A, eSpCas9 1.0), and (K848A/K1003A/R1060A, eSpCas9 1.1) were identified after a deep mutational study focusing on PAM distal mismatches. The two top candidates (K855, K810) found by the EDA method using MM5 were also identified as catalytically important. A recent study by Liu and co-workers proposed two Cas9 variants (HSC 1.1 and HSC 1.2) with enhanced specificity also using a structure-guided engineering method.¹³⁷ The K1246 residue found from the EDA method was also seen in the HSC1.1 variant. The R691A (HiFi Cas9), 138 K526E, R661Q (evoCas9),¹³⁹, and K890N (sniper Cas9)¹⁴⁰ are some of the other residues mentioned in previous studies, which are also aligned with the candidates (Table 6).

TABLE 6
The detailed list of the residues with different allosteric effects
on the Matched and the MM5. The values of ΔE_{Intermol.Interact}.
for each residue are given in parenthesis and the unit is kcal mol⁻¹.

| | E_{Intermol Interact}^{Product} − E_{Intermol Interact}^{Reactant} | | | | E_{Intermol Interact}^{TS} − E_{Intermol Interact}^{Reactant} | | | |
|---|---|---|---|---|---|---|---|---|
| [symbol] s9 | (Thermodynamics of the reaction) | | | | (Kinetics of the reaction) | | | |
| domains | MM5 | | Matched | | MM5 | | Matched | |
| HNH | K810 | (−56.2) | K810 | (61.9) | K810 | (−56.0) | K810 | (59.1) |
| | R859 | (−15.2) | R859 | (8.3) | K855 | (−17.3) | K855 | (48.3) |
| | K855 | (−14.2) | K855 | (51.3) | R859 | (−15.0) | R859 | (8.1) |
| | K896 | (−12.3) | K896 | (21) | K896 | (−12.4) | K896 | (19.6) |
| | R832 | (−11.5) | R832 | (7.0) | R832 | (−11.5) | R832 | (6.7) |
| | R780 | (−10.2) | R780 | (13.6) | R780 | (−10.3) | R780 | (13.4) |
| | R820 | (−9.8) | R820 | (6.1) | K878 | (−9.9) | K878 | (2.8) |
| | K878 | (−9.3) | K878 | (4.0) | R820 | (−9.7) | R820 | (5.7) |
| | K877 | (−9.2) | K877 | (0.0) | R905 | (−8.8) | R905 | (1.1) |
| | R783 | (−7.2) | R783 | (8.3) | K880 | (−7.7) | K880 | (3.2) |
| | K880 | (−7.2) | K880 | (3.7) | R783 | (−7.5) | R783 | (7.9) |
| | K902 | (−7.1) | K902 | (9.7) | K877 | (−7.4) | K877 | (1.9) |
| | R905 | (−6.9) | R905 | (4.4) | K902 | (−7.0) | K902 | (8.5) |
| | R884 | (−6.7) | R884 | (4.1) | R884 | (−6.8) | R884 | (4.0) |
| | K900 | (−6.2) | K900 | (5.6) | K797 | (−6.4) | K797 | (4.1) |
| | K797 | (−6.2) | K797 | (4.3) | K890 | (−6.2) | K890 | (5.1) |
| | K890 | (−6.0) | K890 | (5.6) | R778 | (−5.9) | R778 | (5.5) |
| | K789 | (−6.0) | K789 | (4.8) | K782 | (−5.8) | K782 | (4.8) |
| | R778 | (−5.8) | R778 | (5.7) | K789 | (−5.6) | K789 | (4.6) |
| | K782 | (−5.8) | K782 | (4.9) | K775 | (−5.5) | K775 | (3.3) |
| | K775 | (−5.2) | K775 | (3.5) | R895 | (−4.8) | R895 | (7.7) |
| | R895 | (−4.8) | R895 | (8.5) | K772 | (−4.8) | K772 | (2.5) |
| | K772 | (−3.7) | K772 | (2.5) | K848 | (−3.2) | K848 | (4.1) |
| RuvC-III | R925 | (−5.7) | R925 | (7.2) | R925 | (−5.8) | R925 | (6.9) |
| | K1031 | (−4.5) | K1031 | (1.3) | K1024 | (−4.4) | K1024 | (2.5) |
| | K1024 | (−4.2) | K1024 | (2.9) | K968 | (−4.3) | K968 | (2.2) |
| | K929 | (−4.2) | K929 | (2.5) | K948 | (−4.0) | K948 | (1.7) |
| | K968 | (−4.2) | K968 | (2.4) | K929 | (−3.7) | K929 | (2.3) |
| | K948 | (−4.2) | K948 | (1.7) | K1031 | (−3.2) | K1031 | (1.5) |
| | R976 | (−4.1) | R976 | (2.2) | | | | |
| CTD | R1333 | (−6.7) | R1333 | (0.3) | R1333 | (−7.8) | R1333 | (1.3) |
| | K1325 | (−5.7) | K1325 | (0.5) | R1331 | (−7.8) | R1331 | (1.3) |
| | K1244 | (−5.1) | K1244 | (1.5) | K1325 | (−6.0) | K1325 | (0.6) |
| | H1311 | (−4.9) | H1311 | (0.8) | H1311 | (−5.1) | H1311 | (0.7) |
| | K1246 | (−4.6) | K1246 | (1.2) | K1244 | (−5.1) | K1244 | (1.4) |
| | K1296 | (−4.2) | K1296 | (0.3) | K1296 | (−4.7) | K1296 | (0.4) |
| | K1300 | (−4.1) | K1300 | (0.7) | K1300 | (−4.3) | K1300 | (0.6) |
| L2 | K918 | (−4.8) | K918 | (10.7) | K918 | (−5.1) | K918 | (9.7) |
| | R919 | (−3.3) | R919 | (6.1) | R919 | (−3.2) | R919 | (5.2) |
| REC-I | | | | | F405 | (−5.4) | F405 | (3.3) |
| | | | | | H167 | (−5.2) | H167 | (2.2) |
| REC-II | K268 | (−16.5) | K268 | (5.3) | K268 | (−16.3) | K268 | (5.0) |
| | K218 | (−14.3) | K218 | (2.1) | K218 | (−16.2) | K218 | (2.5) |
| | R221 | (−12.1) | R221 | (1.0) | R221 | (−13.0) | R221 | (1.1) |
| | R220 | (−11.1) | R220 | (3.3) | R220 | (−11.4) | R220 | (4.0) |
| | K263 | (−9.8) | K263 | (5.4) | K263 | (−10.9) | K263 | (8.7) |
| | K234 | (−9.3) | K234 | (5.0) | K253 | (−9.8) | K253 | (7.9) |
| | K253 | (−9.2) | K253 | (7.9) | K209 | (−9.3) | K209 | (1.3) |
| | K209 | (−8.7) | K209 | (2.0) | R629 | (−7.9) | R629 | (1.7) |
| | R307 | (−7.0) | R307 | (0.0) | K234 | (−7.7) | K234 | (4.8) |
| | K233 | (−6.9) | K233 | (4.5) | R653 | (−7.5) | R653 | (4.0) |
| | K294 | (−6.7) | K294 | (1.6) | R307 | (−7.3) | R307 | (0.2) |
| | R629 | (−6.0) | R629 | (0.0) | R655 | (−6.8) | R655 | (3.1) |
| | | | | | R654 | (−6.2) | R654 | (3.5) |
| | | | | | K233 | (−6.2) | K233 | (4.5) |
| | | | | | K294 | (−6.0) | K294 | (0.7) |
| | | | | | K652 | (−5.5) | K652 | (1.9) |
| | | | | | K649 | (−5.5) | K649 | (1.8) |
| | | | | | K646 | (−4.9) | K646 | (1.2) |
| REC-III | R653 | (−7.9) | R653 | (3.8) | R629 | (−7.9) | R629 | (1.7) |
| | R655 | (−6.8) | R655 | (2.5) | R653 | (−7.5) | R653 | (4.0) |
| | R654 | (−6.5) | R654 | (3.4) | R655 | (−6.8) | R655 | (3.1) |
| | R557 | (−6.4) | R557 | (3.8) | R654 | (−6.2) | R654 | (3.5) |
| | K599 | (−6.4) | K599 | (3.6) | K599 | (−6.1) | K599 | (3.7) |
| | R661 | (−6.2) | R661 | (4.5) | R557 | (−6.0) | R557 | (4.1) |
| | K510 | (−6.0) | K510 | (4.2) | K558 | (−5.6) | K558 | (4.3) |
| | H511 | (−6.0) | H511 | (2.9) | K510 | (−5.5) | K510 | (4.7) |
| | K558 | (−5.9) | K558 | (3.5) | R661 | (−5.5) | R661 | (4.6) |
| | K649 | (−5.9) | K649 | (2.3) | H511 | (−5.5) | H511 | (3.4) |
| | R586 | (−5.1) | R586 | (3.6) | K652 | (−5.5) | K652 | (1.9) |
| | K562 | (−4.1) | K562 | (2.0) | K649 | (−5.5) | K649 | (1.8) |
| | K565 | (−4.0) | K565 | (1.8) | R586 | (−5.0) | R586 | (3.7) |
| | R691 | (−3.0)* | R691 | (1.1)* | R635 | (−4.4) | R635 | (1.7) |
| | | | | | K526 | (−4.1) | K526 | (2.5) |
| | | | | | K562 | (−3.9) | K562 | (2.2) |
| | | | | | K500 | (−3.4) | K500 | (3.0) |
| | | | | | R691 | (−3.1)* | R691 | (1.1)* |

[symbol]

indicates data missing or illegible when filed

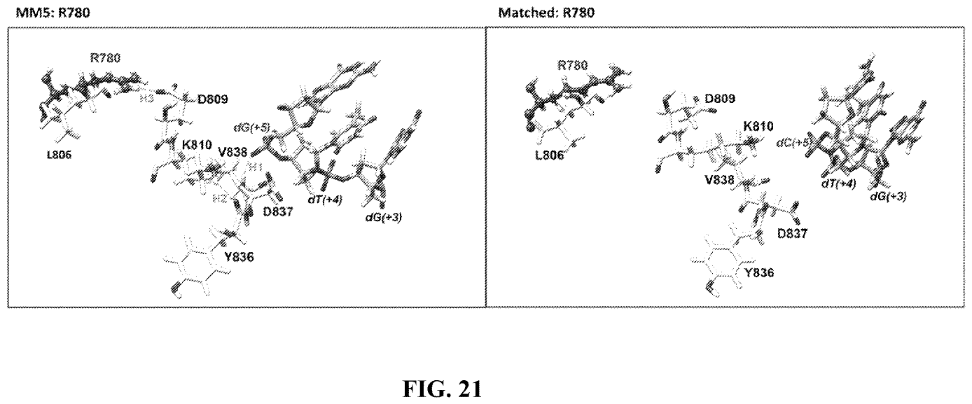

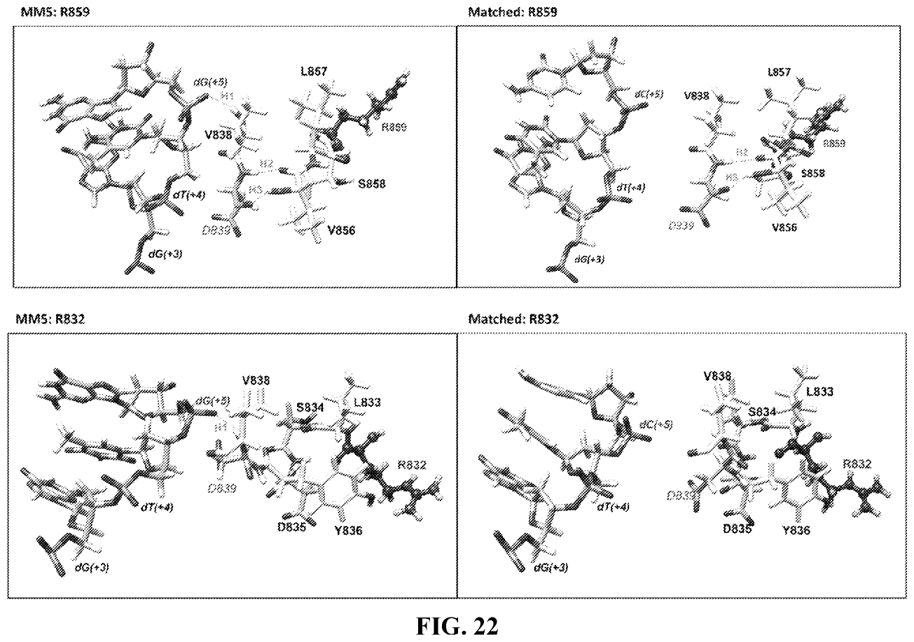

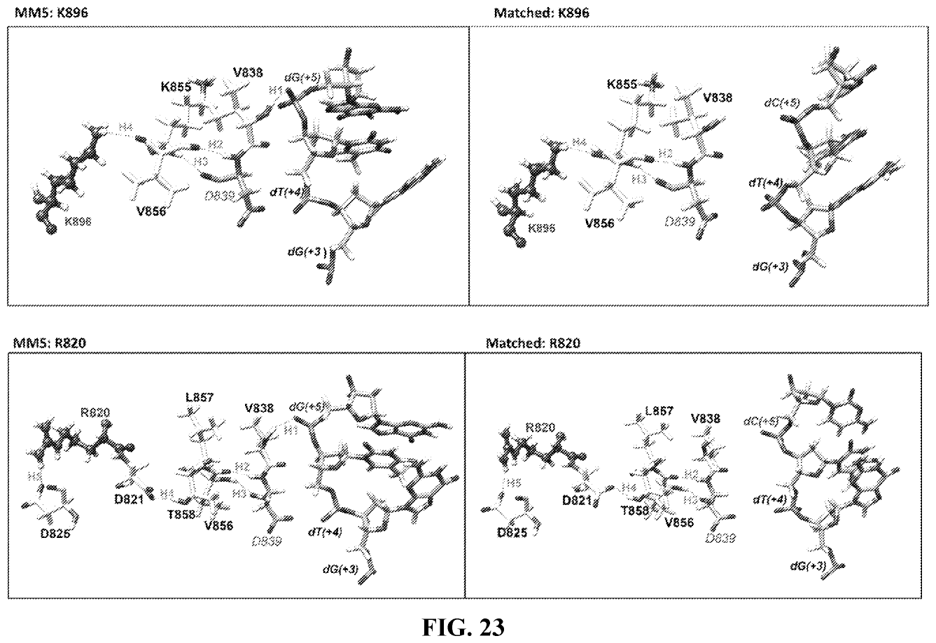

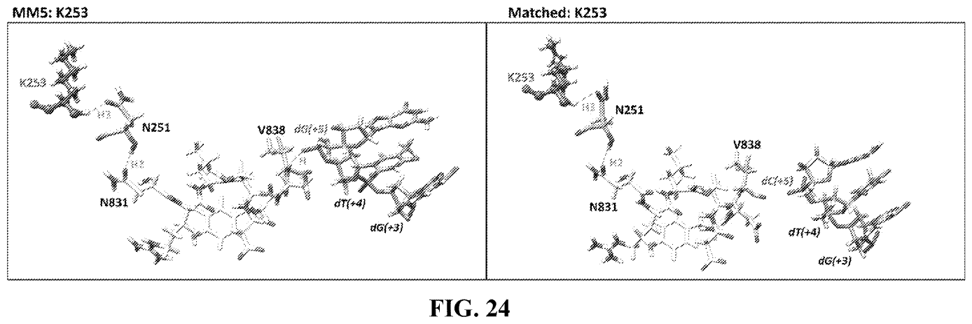

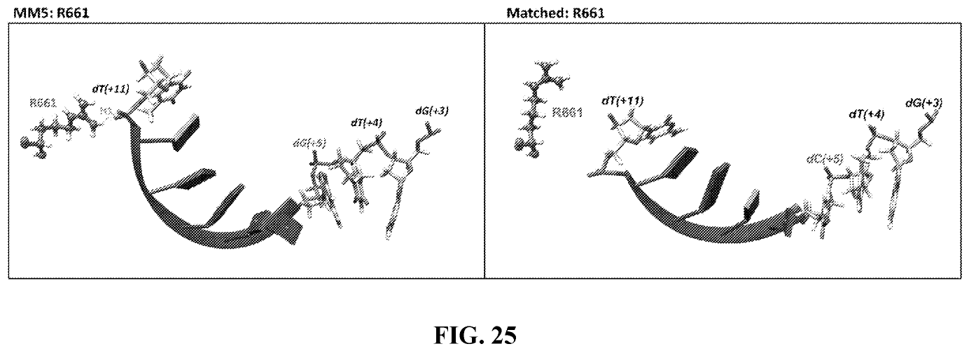

A detailed analysis of the interactions between the HNH active site (including the fifth residue from PAM) and some of the top candidate residues revealed an interesting finding about the stabilization of the MM5 system by those residues. A hydrogen bond between free phosphoryl oxygen of dG(+5) and the backbone of V838 was found to be pivotal to keeping all these candidates connected to the active site through a network of hydrogen bonds for MM5, while it was absent in the matched system (FIG. 21, FIGS. 22-25). Especially, in the case of candidate R780 (FIG. 21), in addition to the hydrogen bond between dG(+5) and V838, interaction between D809 and R⁷⁸⁰ is critical to maintaining the stabilization of MM5 by R780. The same residue mutation to Alanine (R780A) has been shown to work well towards off-target containing CRISPR-Cas9 complexes in the literature.¹³³

The per-residue contribution approach showed herein that there are several residues in the Cas9 that stabilize the HNH catalytic site of both the matched and MM5 systems, while the stabilization effect is higher in the MM5 (Table 7). Further analysis of these residues shows that most residues in the BH region (R63, R66, R69, R70, R74, R75, K76, R78, and K92 in Table 7) contribute to the stability of the MM5 system than matched. Thus, these mutations would destabilize mismatch-containing systems as another approach for these off-target effect removals. Several studies have been focused on these BH's residues mutations in the literature regarding Cas9 specificity, confirming the approach's validity.^{141, 142} The HypaCas9 variant proposed by Chen et al.¹⁴³ involves three amino acid substitutions (N692A/M694A/Q695A/H698A) located on the PAM distal REC-III domain of Cas9. They claim that the mutation of residues within REC-III involved in RNA-DNA heteroduplex recognition, such as those mutated in HypaCas9 or SpCas9-HF1, prevents transitions by the REC-II domain. This more tightly traps the HNH domain in the conformational checkpoint in the presence of mismatches. The EDA approach also revealed several other candidate residues in the REC-III domain (Tables 6 and 7). Although the candidate's selection is based on the PAM proximal single mismatch MM5, the mentioned studies show the credibility of the method and the possible activity of these candidates' mutations towards the other mismatch (especially PAM distal mismatches) containing systems as well. However, further studies are needed to confirm the activities of these mutations concerning their effects on the Cas9 specificity.

TABLE 7
The detailed list of the residues with different allosteric effects
on the Matched and the MM5. The values of ΔE_{Intermol.Interact.} for
each residue are given in parenthesis and the unit is kcal mol⁻¹.

| | E_{Intermol Interact}^{Product} − E_{Intermol Interact}^{Reactant} | | | | E_{Intermol Interact}^{TS} − E_{Intermol Interact}^{Reactant} | | | |
|---|---|---|---|---|---|---|---|---|
| Cas9 | (Thermodynamics of the reaction) | | | | (Kinetics of the reaction) | | | |
| domain [symbol] | MM5 | | Matched | | MM5 | | Matched | |
| BH | R75 | (−12.8) | R75 | (−6.9) | R75 | (−12.4) | R75 | (−5.4) |
| | R78 | (−9.3) | R78 | (−7.1) | R78 | (−9.2) | R78 | (−5.7) |
| | R63 | (−9.0) | R63 | (1.1) | R63 | (−6.3) | R63 | (−1.1) |
| | K76 | (−7.7) | K76 | (−3.7) | K76 | (−7.8) | K76 | (−2.9) |
| | R70 | (−7.2) | R70 | (−4.4) | R70 | (−7.2) | R70 | (−3.6) |
| | R74 | (−6.7) | R74 | (−5.7) | R74 | (−6.7) | R74 | (−5.3) |
| | R66 | (−6.1) | R66 | (−2.9) | R66 | (−5.9) | R66 | (−3.1) |
| | R69 | (−5.4) | R69 | (−4.9) | R69 | (−5.4) | R69 | (−4.7) |
| | K92 | (−4.1) | K92 | (−1.4) | K92 | (−4.2) | K92 | (−1.3) |
| REC-I | R400 | (−20.3) | R400 | (−1.5) | R400 | (−23.5) | R400 | (−1.0) |
| | R403 | (−14.5) | R403 | (−6.6) | R403 | (−14.7) | R403 | (−5.5) |
| | K401 | (−14.3) | K401 | (−6.7) | K401 | (−15.8) | K401 | (−4.9) |
| | K374 | (−12.0) | K374 | (−4.4) | R395 | (−12.5) | R395 | (0.0) |
| | K163 | (−9.7) | K163 | (−5.2) | H415 | (−11.2) | H415 | (−5.0) |
| | K377 | (−9.6) | K377 | (−2.1) | K392 | (−9.5) | K392 | (−1.0) |
| | H415 | (−9.4) | H415 | (−5.6) | K163 | (−9.5) | K163 | (−4.3) |
| | R139 | (−9.3) | R139 | (−3.2) | R139 | (−9.5) | R139 | (−2.8) |
| | R395 | (−9.2) | R395 | (−1.1) | K323 | (−9.2) | K323 | (−1.4) |
| | K392 | (−8.3) | K392 | (−1.4) | K374 | (−9.1) | K374 | (−4.2) |
| | K323 | (−7.4) | K323 | (−1.8) | K377 | (−8.8) | K377 | (−1.4) |
| | H329 | (−6.9) | H329 | (−1.8) | H329 | (−7.4) | H329 | (−1.5) |
| | K336 | (−6.8) | K336 | (−2.3) | H412 | (−7.3) | H412 | (−5.9) |
| | K314 | (−6.6) | K314 | (−0.5) | K336 | (−6.9) | K336 | (−1.8) |
| | K140 | (−6.5) | K140 | (−1.9) | K140 | (−6.8) | K140 | (−1.8) |
| | H129 | (−6.5) | H129 | (−2.6) | H129 | (−6.6) | H129 | (−2.1) |
| | K356 | (−6.5) | K356 | (−3.3) | K442 | (−6.3) | K442 | (−2.3) |
| | K382 | (−6.4) | K382 | (−1.7) | K111 | (−6.3) | K111 | (−2.1) |
| | K111 | (−6.3) | K111 | (−2.5) | R100 | (−5.8) | R100 | (−1.4) |
| | K346 | (−6.1) | K346 | (−2.1) | K141 | (−5.8) | K141 | (−1.4) |
| | K442 | (−5.7) | K442 | (−2.4) | H116 | (−5.6) | H116 | (−1.8) |
| | K141 | (−5.6) | K141 | (−1.9) | K382 | (−5.5) | K382 | (−1.6) |
| | R340 | (−5.4) | R340 | (−1.7) | R437 | (−5.4) | R437 | (−0.4) |
| | R100 | (−5.3) | R100 | (−1.8) | K131 | (−5.1) | K131 | (−1.6) |
| | K439 | (−5.1) | K439 | (−1.6) | R340 | (−5.0) | R340 | (−1.4) |
| | K348 | (−5.1) | K348 | (−2.1) | K439 | (−5.0) | K439 | (−1.4) |
| | K112 | (−5.1) | K112 | (−1.6) | K112 | (−4.9) | K112 | (−1.1) |
| | K131 | (−5.1) | K131 | (−1.8) | R152 | (−4.9) | R152 | (−1.5) |
| | H116 | (−5.0) | H116 | (−2.4) | R115 | (−4.8) | R115 | (−2.7) |
| | R152 | (−4.9) | R152 | (−1.8) | R424 | (−4.8) | R424 | (−1.5) |
| | R424 | (−4.6) | R424 | (−1.4) | K434 | (−4.7) | K434 | (−1.2) |
| | R437 | (−4.5) | R437 | (−0.5) | K148 | (−4.5) | K148 | (−1.0) |
| | K148 | (−4.4) | K148 | (−1.4) | K346 | (−4.3) | K346 | (−1.7) |
| | K434 | (−4.3) | K434 | (−1.0) | K356 | (−4.2) | K356 | (−2.9) |
| | | | | | K348 | (−4.0) | K348 | (−2.2) |
| REC-II | R215 | (−10.2) | R215 | (−5.4) | | | | |
| RuvC-III | K913 | (−5.3) | K913 | (−0.20) | K968 | (−4.2) | K968 | (−1.0) |
| REC-III | R461 | (−6.3) | R461 | (−1.0) | K484 | (−6.2) | K484 | (−0.1) |
| | K484 | (−5.9) | K484 | (0.40) | R461 | (−5.8) | R461 | (−1.0) |
| | R494 | (−4.7) | R494 | (−1.2) | K468 | (−4.6) | K468 | (−0.6) |
| | K468 | (−4.4) | K468 | (−0.7) | R457 | (−4.4) | R457 | (−1.3) |
| | R457 | (−4.4) | R457 | (−1.6) | R494 | (−3.7) | R494 | (−1.3) |
| | R467 | (−4.1) | R467 | (−1.9) | | | | |
| CTD | R1122 | (−8.6) | R1122 | (−4.7) | R1122 | (−7.7) | R1122 | (−3.9) |
| | K1113 | (−6.8) | K1113 | (−2.7) | K1113 | (−6.7) | K1113 | (−2.5) |
| | R1114 | (−5.8) | R1114 | (−2.1) | R1114 | (−6.0) | R1114 | (−1.9) |
| | K1129 | (−5.2) | K1129 | (−2.7) | K1129 | (−5.1) | K1129 | (−1.8) |
| | K1130 | (−5.0) | K1130 | (−2.4) | K1130 | (−5.0) | K1130 | (−2.0) |
| | R1303 | (−4.4) | R1303 | (−1.0) | K1289 | (−4.8) | K1289 | (−0.4) |
| | K1289 | (−4.4) | K1289 | (−0.03) | R1303 | (−4.5) | R1303 | (0.9) |
| | K1334 | (−3.8) | K1334 | (−0.8) | K1334 | (−4.4) | K1334 | (−0.6) |
| | K1192 | (−3.7) | K1192 | (−1.0) | K1192 | (−3.6) | K1192 | (−0.6) |

[symbol]

indicates data missing or illegible when filed

Using a classical molecular dynamics (MD) and hybrid quantum mechanics/molecular mechanics (QM/MM) to study the catalytic cleavage reaction of the t-DNA at the HNH domain of the Cas9 by using a recently discovered catalytic-active structure of this enzyme in complex with RNA and DNA. Based on the MD results, the second-coordination shell water could also be considered the nucleophile in addition to the metal-bound water. To better understand the impact of sg-RNA and t-DNA complementarity on the catalysis process, a mismatched structure (MM5) was designed with a C to G mismatch at the fifth position of the t-DNA's PAM region. Calculated QM/MM results show that the nucleophilic attack by the second-coordination shell water with the reaction energy of 32.6 kcal mol⁻¹ is not energetically feasible. Calculated reaction energies for the matched and MM5 with the attacking water bound to the Mg²⁺ ion are −31.3 and −10.16 kcal mol⁻¹, which shows the structural effect of the t-DNA mismatch on the catalytic function of the Cas9. Additionally, the ESP charges of the attacking water and its non-covalent interactions with the active site residues show that the reactant of the matched is more favorable than the MM5. In agreement with the QM/MM energy barriers and reaction energies for the matched and MM5, results of the energy decomposition analysis (EDA) show that the non-bonded intermolecular interactions between the Cas9 and the residues of the active site in the TS and product of the matched are considerably more stabilizing than the MM5. This shows that the amino acid residues of the Cas9 have stabilizing contributions in the reactant-TS path, but this facilitating contribution is significantly larger in the matched structure. The EDA results also show that residues K253, R780, R783, K810, R832, K855, R859, K896, and K902 can be good targets for the mutation. In particular, K253 and K896 of the REC-II and HNH regions are of particular interest.

### REFERENCES

1. Makarova, K. S.; Grishin, N. V.; Shabalina, S. A.; Wolf, Y. I.; Koonin, E. V. J. B. d., A putative RNA-interference-based immune system in prokaryotes: computational analysis of the predicted enzymatic machinery, functional analogies with eukaryotic RNAi, and hypothetical mechanisms of action. 2006, 1 (1), 1-26.
2. Jinek, M.; Jiang, F.; Taylor, D. W.; Sternberg, S. H.; Kaya, E.; Ma, E.; Anders, C.; Hauer, M.; Zhou, K.; Lin, S. J. S., Structures of Cas9 endonucleases reveal RNA-mediated conformational activation. 2014, 343 (6176).
3. Ishino, Y.; Shinagawa, H.; Makino, K.; Amemura, M.; Nakata, A. J. J. o. b., Nucleotide sequence of the iap gene, responsible for alkaline phosphatase isozyme conversion in Escherichia coli, and identification of the gene product. 1987, 169 (12), 5429-5433.
4. Bolotin, A.; Quinquis, B.; Sorokin, A.; Ehrlich, S. D. J. M., Clustered regularly interspaced short palindrome repeats (CRISPRs) have spacers of extrachromosomal origin. 2005, 151 (8), 2551-2561.
5. Brouns, S. J.; Jore, M. M.; Lundgren, M.; Westra, E. R.; Slijkhuis, R. J.; Snijders, A. P.; Dickman, M. J.; Makarova, K. S.; Koonin, E. V.; Van Der Oost, J. J. S., Small CRISPR RNAs guide antiviral defense in prokaryotes. 2008, 321 (5891), 960-964.
6. Mojica, F. J.; Díez-Villaseñor, C.; García-Martínez, J.; Almendros, C. J. M., Short motif sequences determine the targets of the prokaryotic CRISPR defence system. 2009, 155 (3), 733-740.
7. Terns, M. P.; Terns, R. M., CRISPR-based adaptive immune systems. Current Opinion in Microbiology 2011, 14 (3), 321-327.
8. Gasiunas, G.; Barrangou, R.; Horvath, P.; Siksnys, V. J. P. o. t. N. A. o. S., Cas9-crRNA ribonucleoprotein complex mediates specific DNA cleavage for adaptive immunity in bacteria. 2012, 109 (39), E2579-E2586.
9. Koonin, E. V.; Makarova, K. S. J. P. T. o. t. R. S. B., Origins and evolution of CRISPR-Cas systems. 2019, 374 (1772), 20180087.
10. Mojica, F. J. M.; Montoliu, L., On the Origin of CRISPR-Cas Technology: From Prokaryotes to Mammals. Trends in Microbiology 2016, 24 (10), 811-820.
11. Ding, Y.; Li, H.; Chen, L.-L.; Xie, K. J. F. i. p. s., Recent advances in genome editing using CRISPR/Cas9. 2016, 7, 703.
12. Wang, F.; Wang, L.; Zou, X.; Duan, S.; Li, Z.; Deng, Z.; Luo, J.; Lee, S. Y.; Chen, S. J. B. a., Advances in CRISPR-Cas systems for RNA targeting, tracking and editing. 2019, 37 (5), 708-729.
13. Pickar-Oliver, A.; Gersbach, C. A. J. N. r. M. c. b., The next generation of CRISPR-Cas technologies and applications. 2019, 20 (8), 490-507.
14. Yip, B. H. J. B., Recent advances in CRISPR/Cas9 delivery strategies. 2020, 10 (6), 839.
15. Wei, T.; Cheng, Q.; Farbiak, L.; Anderson, D. G.; Langer, R.; Siegwart, D. J. J. A. n., Delivery of tissue-targeted scalpels: opportunities and challenges for in vivo CRISPR/Cas-based genome editing. 2020, 14 (8), 9243-9262.
16. Goell, J. H.; Hilton, I. B. J. T. i. B., CRISPR/Cas-based epigenome editing: advances, applications, and clinical utility. 2021, 39 (7), 678-691.
17. Rao, M. J.; Wang, L. J. P., CRISPR/Cas9 technology for improving agronomic traits and future prospective in agriculture. 2021, 254 (4), 1-16.
18. Makarova, K. S.; Wolf, Y. I.; Alkhnbashi, O. S.; Costa, F.; Shah, S. A.; Saunders, S. J.; Barrangou, R.; Brouns, S. J. J.; Charpentier, E.; Haft, D. H.; Horvath, P.; Moineau, S.; Mojica, F. J. M.; Terns, R. M.; Terns, M. P.; White, M. F.; Yakunin, A. F.; Garrett, R. A.; van der Oost, J.; Backofen, R.; Koonin, E. V., An updated evolutionary classification of CRISPR-Cas systems. Nature Reviews Microbiology 2015, 13 (11), 722-736.
19. Andersson, A. F.; Banfield, J. F. J. S., Virus population dynamics and acquired virus resistance in natural microbial communities. 2008, 320 (5879), 1047-1050.
20. Jinek, M.; Chylinski, K.; Fonfara, I.; Hauer, M.; Doudna, J. A.; Charpentier, E. J. s., A programmable dual-RNA-guided DNA endonuclease in adaptive bacterial immunity. 2012, 337 (6096), 816-821.
21. Koonin, E. V.; Makarova, K. S.; Zhang, F. J. C. o. i. m., Diversity, classification and evolution of CRISPR-Cas systems. 2017, 37, 67-78.
22. Makarova, K. S.; Wolf, Y. I.; Iranzo, J.; Shmakov, S. A.; Alkhnbashi, O. S.; Brouns, S. J.; Charpentier, E.; Cheng, D.; Haft, D. H.; Horvath, P. J. N. R. M., Evolutionary classification of CRISPR-Cas systems: a burst of class 2 and derived variants. 2020, 18 (2), 67-83.
23. Cong, L.; Ran, F. A.; Cox, D.; Lin, S.; Barretto, R.; Habib, N.; Hsu, P. D.; Wu, X.; Jiang, W.; Marraffini, L. A. J. S., Multiplex genome engineering using CRISPR/Cas systems. 2013, 339 (6121), 819-823.
24. Jinek, M.; East, A.; Cheng, A.; Lin, S.; Ma, E.; Doudna, J. J. e., RNA-programmed genome editing in human cells. 2013, 2, e00471.
25. Konermann, S.; Brigham, M. D.; Trevino, A. E.; Joung, J.; Abudayyeh, O. O.; Barcena, C.; Hsu, P. D.; Habib, N.; Gootenberg, J. S.; Nishimasu, H. J. N., Genome-scale transcriptional activation by an engineered CRISPR-Cas9 complex. 2015, 517 (7536), 583-588.
26. Murugan, K.; Babu, K.; Sundaresan, R.; Rajan, R.; Sashital, D. G. J. M. c., The revolution continues: newly discovered systems expand the CRISPR-Cas toolkit. 2017, 68 (1), 15-25.
27. Fogarty, N. M. E.; McCarthy, A.; Snijders, K. E.; Powell, B. E.; Kubikova, N.; Blakeley, P.; Lea, R.; Elder, K.; Wamaitha, S. E.; Kim, D.; Maciulyte, V.; Kleinjung, J.; Kim, J.-S.; Wells, D.; Vallier, L.; Bertero, A.; Turner, J. M. A.; Niakan, K. K., Genome editing reveals a role for OCT4 in human embryogenesis. Nature 2017, 550 (7674), 67-73.
28. Adli, M. J. N. c., The CRISPR tool kit for genome editing and beyond. 2018, 9 (1), 1-13.
29. Jinek, M., Krzysztof; Chylinski, Ines; Fonfara, M; Hauer, Jennifer; Doudna, and Emmanuelle Charpentier. 2012. “A Programmable Dual-RNA-Guided DNA Endonuclease in Adaptive Bacterial Immunity.”. Science (337), 816-21.
30. Jiang, F.; Zhou, K.; Ma, L.; Gressel, S.; Doudna, J. A., A Cas9-guide RNA complex preorganized for target DNA recognition. Science 2015, 348 (6242), 1477-1481.
31. Geny, S.; Pichard, S.; Brion, A.; Renaud, J.-B.; Jacquemin, S.; Concordet, J.-P.; Poterszman, A., Tagging Proteins with Fluorescent Reporters Using the CRISPR/Cas9 System and Double-Stranded DNA Donors. In Multiprotein Complexes, Springer: 2021; pp 39-57.
32. Fu, Y.; Foden, J. A.; Khayter, C.; Maeder, M. L.; Reyon, D.; Joung, J. K.; Sander, J. D., High-frequency off-target mutagenesis induced by CRISPR-Cas nucleases in human cells. Nature biotechnology 2013, 31 (9), 822-826.
33. Tsai, S. Q.; Joung, J. K., Defining and improving the genome-wide specificities of CRISPR-Cas9 nucleases. Nature Reviews Genetics 2016, 17 (5), 300-312.
34. Wu, S.-S.; Li, Q.-C.; Yin, C.-Q.; Xue, W.; Song, C.-Q., Advances in CRISPR/Cas-based gene therapy in human genetic diseases. Theranostics 2020, 10 (10), 4374.
35. Dagdas, Y. S.; Chen, J. S.; Sternberg, S. H.; Doudna, J. A.; Yildiz, A., A conformational checkpoint between DNA binding and cleavage by CRISPR-Cas9. Science advances 2017, 3 (8), eaao0027.
36. Chen, J. S.; Dagdas, Y. S.; Kleinstiver, B. P.; Welch, M. M.; Sousa, A. A.; Harrington, L. B.; Sternberg, S. H.; Joung, J. K.; Yildiz, A.; Doudna, J. A., Enhanced proofreading governs CRISPR-Cas9 targeting accuracy. Nature 2017, 550 (7676), 407-410.
37. Anders, C.; Niewoehner, O.; Duerst, A.; Jinek, M., Structural basis of PAM-dependent target DNA recognition by the Cas9 endonuclease. Nature 2014, 513 (7519), 569-573.
38. Nishimasu, H.; Ran, F. A.; Hsu, Patrick D.; Konermann, S.; Shehata, Soraya I.; Dohmae, N.; Ishitani, R.; Zhang, F.; Nureki, O., Crystal Structure of Cas9 in Complex with Guide RNA and Target DNA. Cell 2014, 156 (5), 935-949.
39. Anders, C.; Bargsten, K.; Jinek, M., Structural Plasticity of PAM Recognition by Engineered Variants of the RNA-Guided Endonuclease Cas9. Molecular Cell 2016, 61 (6), 895-902.
40. Dong, D.; Guo, M.; Wang, S.; Zhu, Y.; Wang, S.; Xiong, Z.; Yang, J.; Xu, Z.; Huang, Z., Structural basis of CRISPR-SpyCas9 inhibition by an anti-CRISPR protein. Nature 2017, 546 (7658), 436-439.
41. Yang, H.; Patel, D. J., Inhibition Mechanism of an Anti-CRISPR Suppressor AcrIIA4 Targeting SpyCas9. Molecular Cell 2017, 67 (1), 117-127.e5.
42. Liu, L.; Yin, M.; Wang, M.; Wang, Y., Phage AcrIIA2 DNA Mimicry: Structural Basis of the CRISPR and Anti-CRISPR Arms Race. Molecular Cell 2019, 73 (3), 611-620.e3.
43. Huai, C.; Li, G.; Yao, R.; Zhang, Y.; Cao, M.; Kong, L.; Jia, C.; Yuan, H.; Chen, H.; Lu, D.; Huang, Q., Structural insights into DNA cleavage activation of CRISPR-Cas9 system. Nature Communications 2017, 8 (1), 1375.
44. Shin, J.; Jiang, F.; Liu, J.-J.; Bray, N. L.; Rauch, B. J.; Baik, S. H.; Nogales, E.; Bondy-Denomy, J.; Corn, J. E.; Doudna, J. A., Disabling Cas9 by an anti-CRISPR DNA mimic. 2017, 3 (7), e1701620.
45. Jiang, F.; Liu, J.-J.; Osuna, B. A.; Xu, M.; Berry, J. D.; Rauch, B. J.; Nogales, E.; Bondy-Denomy, J.; Doudna, J. A., Temperature-Responsive Competitive Inhibition of CRISPR-Cas9. Molecular Cell 2019, 73 (3), 601-610.e5.
46. Zuo, Z.; Liu, J., Structure and Dynamics of Cas9 HNH Domain Catalytic State. Scientific Reports 2017, 7 (1), 17271.
47. Palermo, G.; Chen, J. S.; Ricci, C. G.; Rivalta, I.; Jinek, M.; Batista, V. S.; Doudna, J. A.; McCammon, J. A. J. Q. r. o. b., Key role of the REC lobe during CRISPR-Cas9 activation by ‘sensing’, ‘regulating’, and ‘locking’ the catalytic HNH domain. 2018, 51.
48. Palermo, G., Structure and Dynamics of the CRISPR-Cas9 Catalytic Complex. Journal of Chemical Information and Modeling 2019, 59 (5), 2394-2406.
49. Cotton, F. A.; Hazen, E. E.; Legg, M. J. J. P. o. t. N. A. o. S., Staphylococcal nuclease: Proposed mechanism of action based on structure of enzyme-thymidine 3′, 5′-bisphosphate-calcium ion complex at 1.5-Å resolution. 1979, 76 (6), 2551-2555.
50. Li, C.-L.; Hor, L.-I.; Chang, Z.-F.; Tsai, L.-C.; Yang, W.-Z.; Yuan, H. S. J. T. E. j., DNA binding and cleavage by the periplasmic nuclease Vvn: a novel structure with a known active site. 2003, 22 (15), 4014-4025.
51. Biertümpfel, C.; Yang, W.; Suck, D. J. N., Crystal structure of T4 endonuclease VII resolving a Holliday junction. 2007, 449 (7162), 616-620.
52. Yang, W. J. N. s.; biology, m., An equivalent metal ion in one- and two-metal-ion catalysis. 2008, 15 (11), 1228-1231.
53. Yang, W. J. Q. r. o. b., Nucleases: diversity of structure, function and mechanism. 2011, 44 (1), 1-93.
54. Yoon, H.; Zhao, L. N.; Warshel, A. J. A. c., Exploring the catalytic mechanism of Cas9 using information inferred from endonuclease VII. 2018, 9 (2), 1329-1336.
55. Zhao, L. N.; Mondal, D.; Warshel, A., Exploring alternative catalytic mechanisms of the Cas9 HNH domain. Proteins: Structure, Function, and Bioinformatics 2020, 88 (2), 260-264.
56. Zhu, X.; Clarke, R.; Puppala, A. K.; Chittori, S.; Merk, A.; Merrill, B. J.; Simonović, M.; Subramaniam, S. J. N. s.; biology, m., Cryo-EM structures reveal coordinated domain motions that govern DNA cleavage by Cas9. 2019, 26 (8), 679-685.
57. Zuo, Z.; Zolekar, A.; Babu, K.; Lin, V. J. T.; Hayatshahi, H. S.; Rajan, R.; Wang, Y.-C.; Liu, J., Structural and functional insights into the bona fide catalytic state of Streptococcus pyogenes Cas9 HNH nuclease domain. eLife 2019, 8, e46500.
58. Jiang, F.; Taylor, D. W.; Chen, J. S.; Kornfeld, J. E.; Zhou, K.; Thompson, A. J.; Nogales, E.; Doudna, J. A., Structures of a CRISPR-Cas9 R-loop complex primed for DNA cleavage. Science 2016, 351 (6275), 867-871.
59. Jinek, M.; Jiang, F.; Taylor, D. W.; Sternberg, S. H.; Kaya, E.; Ma, E.; Anders, C.; Hauer, M.; Zhou, K.; Lin, S.; Kaplan, M.; Iavarone, A. T.; Charpentier, E.; Nogales, E.; Doudna, J. A., Structures of Cas9 Endonucleases Reveal RNA-Mediated Conformational Activation. Science 2014, 343 (6176), 1247997.
60. Schafmeister, C.; Ross, W.; Romanovski, V., LEaP. University of California, San Francisco 1995.
61. Jorgensen, W. L.; Chandrasekhar, J.; Madura, J. D.; Impey, R. W.; Klein, M. L., Comparison of simple potential functions for simulating liquid water. The Journal of chemical physics 1983, 79 (2), 926-935.
62. Maier, J. A.; Martinez, C.; Kasavajhala, K.; Wickstrom, L.; Hauser, K. E.; Simmerling, C., ff14SB: improving the accuracy of protein side chain and backbone parameters from ff99SB. Journal of chemical theory and computation 2015, 11 (8), 3696-3713.
63. Galindo-Murillo, R.; Robertson, J. C.; Zgarbová, M.; Sponer, J.; Otyepka, M.; Jurecka, P.; Cheatham III, T. E., Assessing the current state of amber force field modifications for DNA. Journal of chemical theory and computation 2016, 12 (8), 4114-4127.

64 Zgarbová, M.; Otyepka, M.; Šponer, J. i.; Mládek, A. t.; Banáš, P.; Cheatham III, T. E.; Jurecka, P., Refinement of the Cornell et al. nucleic acids force field based on reference quantum chemical calculations of glycosidic torsion profiles. Journal of chemical theory and computation 2011, 7 (9), 2886-2902.
65. Case, D.; Ben-Shalom, I.; Brozell, S.; Cerutti, D.; Cheatham III, T.; Cruzeiro, V.; Darden, T.; Duke, R.; Ghoreishi, D.; Gilson, M., AMBER 2018, Univ. California, San Fr 2018.
66. Zwanzig, R., Nonlinear generalized Langevin equations. Journal of Statistical Physics 1973, 9 (3), 215-220.
67. Loncharich, R. J.; Brooks, B. R.; Pastor, R. W., Langevin dynamics of peptides: The frictional dependence of isomerization rates of N-acetylalanyl-N′-methylamide. Biopolymers: Original Research on Biomolecules 1992, 32 (5), 523-535.
68. Gillespie, D. T., The chemical Langevin equation. The Journal of Chemical Physics 2000, 113 (1), 297-306.
69. Ryckaert, J.-P.; Ciccotti, G.; Berendsen, H. J., Numerical integration of the cartesian equations of motion of a system with constraints: molecular dynamics of n-alkanes. Journal of computational physics 1977, 23 (3), 327-341.
70. Essmann, U.; Perera, L.; Berkowitz, M. L.; Darden, T.; Lee, H.; Pedersen, L. G., A smooth particle mesh Ewald method. The Journal of chemical physics 1995, 103 (19), 8577-8593.
71. Salomon-Ferrer, R.; Gotz, A. W.; Poole, D.; Le Grand, S.; Walker, R. C., Routine microsecond molecular dynamics simulations with AMBER on GPUs. 2. Explicit solvent particle mesh Ewald. Journal of chemical theory and computation 2013, 9 (9), 3878-3888.
72. Roe, D. R.; Cheatham III, T. E., PTRAJ and CPPTRAJ: software for processing and analysis of molecular dynamics trajectory data. Journal of chemical theory and computation 2013, 9 (7), 3084-3095.
73. Likas, A.; Vlassis, N.; Verbeek, J. J., The global k-means clustering algorithm. Pattern recognition 2003, 36 (2), 451-461.
74. Kollman, P. A.; Massova, I.; Reyes, C.; Kuhn, B.; Huo, S.; Chong, L.; Lee, M.; Lee, T.; Duan, Y.; Wang, W., Calculating structures and free energies of complex molecules: combining molecular mechanics and continuum models. Accounts of chemical research 2000, 33 (12), 889-897.
75. Wang, W.; Donini, O.; Reyes, C. M.; Kollman, P. A., Biomolecular simulations: recent developments in force fields, simulations of enzyme catalysis, protein-ligand, protein-protein, and protein-nucleic acid noncovalent interactions. Annual review of biophysics and biomolecular structure 2001, 30 (1), 211-243.
76. Wang, J.; Hou, T.; Xu, X., Recent advances in free energy calculations with a combination 76. of molecular mechanics and continuum models. Current Computer-Aided Drug Design 2006, 2 (3), 287-306.
77. Homeyer, N.; Gohlke, H., Free energy calculations by the molecular mechanics Poisson-Boltzmann surface area method. Molecular informatics 2012, 31 (2), 114-122.
78. Miller III, B. R.; McGee Jr, T. D.; Swails, J. M.; Homeyer, N.; Gohlke, H.; Roitberg, A. E., MMPBSA. py: an efficient program for end-state free energy calculations. Journal of chemical theory and computation 2012, 8 (9), 3314-3321.
79. Li, C. H.; Zuo, Z. C.; Su, J. G.; Xu, X. J.; Wang, C. X., The interactions and recognition of cyclic peptide mimetics of Tat with HIV-1 TAR RNA: a molecular dynamics simulation study. Journal of Biomolecular Structure and Dynamics 2013, 31 (3), 276-287.
80. Zuo, Z.; Liu, J., Cas9-catalyzed DNA cleavage generates staggered ends: evidence from molecular dynamics simulations. Scientific reports 2016, 6 (1), 1-9.
81. Zuo, Z.; Weng, J.; Wang, W., Insights into the inhibitory mechanism of D13-9001 to the multidrug transporter AcrB through molecular dynamics simulations. The Journal of Physical Chemistry B 2016, 120 (9), 2145-2154.
82. Zuo, Z.; Smith, R. N.; Chen, Z.; Agharkar, A. S.; Snell, H. D.; Huang, R.; Liu, J.; Gonzales, E. B., Identification of a unique Ca2+-binding site in rat acid-sensing ion channel 3. Nature communications 2018, 9 (1), 1-11.
83. Zuo, Z.; Liu, J., Structure and dynamics of Cas9 HNH domain catalytic state. Scientific reports 2017, 7 (1), 1-13.
84. Naseem-Khan, S.; Berger, M. B.; Leddin, E. M.; Maghsoud, Y.; Cisneros, G. A., Impact of Remdesivir Incorporation along the Primer Strand on SARS-COV-2 RNA-Dependent RNA Polymerase. Journal of Chemical Information and Modeling 2022, 62 (10), 2456-2465.
85. Kratz, E. G.; Walker, A. R.; Lagardère, L.; Lipparini, F.; Piquemal, J. P.; Andrés Cisneros, G., LICHEM: A QM/MM program for simulations with multipolar and polarizable force fields. Journal of computational chemistry 2016, 37 (11), 1019-1029.
86. Gökcan, H.; Vázquez-Montelongo, E. A.; Cisneros, G. A., LICHEM 1.1: recent improvements and new capabilities. Journal of chemical theory and computation 2019, 15 (5), 3056-3065.
87. Frisch, M. J.; Trucks, G. W.; Schlegel, H. B.; Scuseria, G. E.; Robb, M. A.; Cheeseman, J. R.; Scalmani, G.; Barone, V.; Petersson, G. A.; Nakatsuji, H.; Li, X.; Caricato, M.; Marenich, A. V.; Bloino, J.; Janesko, B. G.; Gomperts, R.; Mennucci, B.; Hratchian, H. P.; Ortiz, J. V.; Izmaylov, A. F.; Sonnenberg, J. L.; Williams; Ding, F.; Lipparini, F.; Egidi, F.; Goings, J.; Peng, B.; Petrone, A.; Henderson, T.; Ranasinghe, D.; Zakrzewski, V. G.; Gao, J.; Rega, N.; Zheng, G.; Liang, W.; Hada, M.; Ehara, M.; Toyota, K.; Fukuda, R.; Hasegawa, J.; Ishida, M.; Nakajima, T.; Honda, Y.; Kitao, O.; Nakai, H.; Vreven, T.; Throssell, K.; Montgomery Jr., J. A.; Peralta, J. E.; Ogliaro, F.; Bearpark, M. J.; Heyd, J. J.; Brothers, E. N.; Kudin, K. N.; Staroverov, V. N.; Keith, T. A.; Kobayashi, R.; Normand, J.; Raghavachari, K.; Rendell, A. P.; Burant, J. C.; Iyengar, S. S.; Tomasi, J.; Cossi, M.; Millam, J. M.; Klene, M.; Adamo, C.; Cammi, R.; Ochterski, J. W.; Martin, R. L.; Morokuma, K.; Farkas, O.; Foresman, J. B.; Fox, D. J. Gaussian 16 Rev. C.01, Wallingford, CT, 2016.
88. Rackers, J. A.; Wang, Z.; Lu, C.; Laury, M. L.; Lagardère, L.; Schnieders, M. J.; Piquemal, J.-P.; Ren, P.; Ponder, J. W., Tinker 8: software tools for molecular design. Journal of chemical theory and computation 2018, 14 (10), 5273-5289.
89. Chai, J.-D.; Head-Gordon, M., Long-range corrected hybrid density functionals with damped atom-atom dispersion corrections. Physical Chemistry Chemical Physics 2008, 10 (44), 6615-6620.
90. Chai, J.-D.; Head-Gordon, M., Systematic optimization of long-range corrected hybrid density functionals. The Journal of chemical physics 2008, 128 (8), 084106.
91. Kratz, E. G.; Duke, R. E.; Cisneros, G. A., Long-range electrostatic corrections in multipolar/polarizable QM/MM simulations. Theoretical chemistry accounts 2016, 135 (7), 1-9.
92. Fang, D.; Chaudret, R.; Piquemal, J.-P.; Cisneros, G. A. s., Toward a deeper understanding of enzyme reactions using the coupled ELF/NCI analysis: application to DNA repair enzymes. Journal of Chemical Theory and Computation 2013, 9 (5), 2156-2160.
93. Johnson, E. R.; Keinan, S.; Mori-Sánchez, P.; Contreras-García, J.; Cohen, A. J.; Yang, W., Revealing noncovalent interactions. Journal of the American Chemical Society 2010, 132 (18), 6498-6506.
94 Lu, T.; Chen, F., Multiwfn: a multifunctional wavefunction analyzer. Journal of computational chemistry 2012, 33 (5), 580-592.
95. Swails, J.; Hernandez, C.; Mobley, D. L.; Nguyen, H.; Wang, L.-P.; Janowski, P., ParmEd. URL: https://github.com/ParmEd/ParmEd 2010.
96. Bayly, C. I.; Cieplak, P.; Cornell, W.; Kollman, P. A., A well-behaved electrostatic potential based method using charge restraints for deriving atomic charges: the RESP model. The Journal of Physical Chemistry 1993, 97 (40), 10269-10280.
97 Dupradeau, F.-Y.; Pigache, A.; Zaffran, T.; Savineau, C.; Lelong, R.; Grivel, N.; Lelong, D.; Rosanski, W.; Cieplak, P., The REd. Tools: Advances in RESP and ESP charge derivation and force field library building. Physical Chemistry Chemical Physics 2010, 12 (28), 7821-7839.
98. Vanquelef, E.; Simon, S.; Marquant, G.; Garcia, E.; Klimerak, G.; Delepine, J. C.; Cieplak, P.; Dupradeau, F.-Y., RED Server: a web service for deriving RESP and ESP charges and building force field libraries for new molecules and molecular fragments. Nucleic acids research 2011, 39 (suppl_2), W511-W517.
99. Wang, F.; Becker, J.-P.; Cieplak, P.; Dupradeau, F.-Y. In RED Python: Object oriented programming for Amber force fields, Abstracts of Papers of the American Chemical Society, AMER CHEMICAL SOC 1155 16TH ST, NW, WASHINGTON, DC 20036 USA: 2014.
100. Wang, J.; Wolf, R. M.; Caldwell, J. W.; Kollman, P. A.; Case, D. A., Development and testing of a general amber force field. Journal of computational chemistry 2004, 25 (9), 1157-1174.
101. Wang, J.; Wang, W.; Kollman, P. A.; Case, D. A., Automatic atom type and bond type perception in molecular mechanical calculations. Journal of molecular graphics and modelling 2006, 25 (2), 247-260.
102. Graham, S. E.; Syeda, F.; Cisneros, G. A. s., Computational prediction of residues involved in fidelity checking for DNA synthesis in DNA polymerase I. Biochemistry 2012, 51 (12), 2569-2578.
103. Dewage, S. W.; Cisneros, G. A., Computational analysis of ammonia transfer along two intramolecular tunnels in Staphylococcus aureus glutamine-dependent amidotransferase (GatCAB). The Journal of Physical Chemistry B 2015, 119 (9), 3669-3677.
104. Walker, A. R.; Cisneros, G. A. s., Computational simulations of DNA polymerases: detailed insights on structure/function/mechanism from native proteins to cancer variants. Chemical research in toxicology 2017, 30 (11), 1922-1935.
105. Cui, Q.; Karplus, M., Catalysis and specificity in enzymes: a study of triosephosphate isomerase and comparison with methyl glyoxal synthase. Advances in protein chemistry 2003, 66, 315-372.
106. Martí, S.; Andrés, J.; Moliner, V.; Silla, E.; Tuñón, I.; Bertrán, J., Preorganization and reorganization as related factors in enzyme catalysis: the chorismate mutase case. Chemistry-A European Journal 2003, 9 (4), 984-991.
107. Senn, H. M.; O'Hagan, D.; Thiel, W., Insight into enzymatic C—F Bond formation from QM and QM/MM calculations. Journal of the American Chemical Society 2005, 127 (39), 13643-13655.
108. Cisneros, G. A.; Perera, L.; Schaaper, R. M.; Pedersen, L. C.; London, R. E.; Pedersen, L. G.; Darden, T. A., Reaction mechanism of the & subunit of E. coli DNA polymerase III: insights into active site metal coordination and catalytically significant residues. Journal of the American Chemical Society 2009, 131 (4), 1550-1556.
109. Fang, D.; Lord, R. L.; Cisneros, G. A., Ab initio QM/MM calculations show an intersystem crossing in the hydrogen abstraction step in dealkylation catalyzed by AlkB. The Journal of Physical Chemistry B 2013, 117 (21), 6410-6420.
110. Fang, D.; Cisneros, G. A. s., Alternative pathway for the reaction catalyzed by DNA dealkylase AlkB from ab initio QM/MM calculations. Journal of chemical theory and computation 2014, 10 (11), 5136-5148.
111. Torabifard, H.; Cisneros, G. A., Insight into wild-type and T1372E TET2-mediated 5hmC oxidation using ab initio QM/MM calculations. Chemical science 2018, 9 (44), 8433-8445.
112. Pettersen, E. F.; Goddard, T. D.; Huang, C. C.; Couch, G. S.; Greenblatt, D. M.; Meng, E. C.; Ferrin, T. E., UCSF Chimera-A visualization system for exploratory research and analysis. Journal of Computational Chemistry 2004, 25 (13), 1605-1612.
113. Humphrey, W.; Dalke, A.; Schulten, K., VMD: visual molecular dynamics. Journal of molecular graphics 1996, 14 (1), 33-38.
114. Dennington, R.; Keith, T. A.; Millam, J. M., Gauss View 6.0. 16. Semichem Inc.: Shawnee Mission, KS, USA 2016.
115. Jinek, M.; Chylinski, K.; Fonfara, I.; Hauer, M.; Doudna, J. A.; Charpentier, E., A programmable dual-RNA-guided DNA endonuclease in adaptive bacterial immunity. science 2012, 337 (6096), 816-821.
116. Sternberg, S. H.; Redding, S.; Jinek, M.; Greene, E. C.; Doudna, J. A., DNA interrogation by the CRISPR RNA-guided endonuclease Cas9. Nature 2014, 507 (7490), 62-67.
117. Sternberg, S. H.; LaFrance, B.; Kaplan, M.; Doudna, J. A., Conformational control of DNA target cleavage by CRISPR-Cas9. Nature 2015, 527 (7576), 110-113.
118. Singh, D.; Sternberg, S. H.; Fei, J.; Doudna, J. A.; Ha, T., Real-time observation of DNA recognition and rejection by the RNA-guided endonuclease Cas9. Nature communications 2016, 7 (1), 1-8.
119. Jiang, F.; Doudna, J. A., CRISPR-Cas9 structures and mechanisms. Annu Rev Biophys 2017, 46 (1), 505-529.
120. Singh, D.; Wang, Y.; Mallon, J.; Yang, O.; Fei, J.; Poddar, A.; Ceylan, D.; Bailey, S.; Ha, T., Mechanisms of improved specificity of engineered Cas9s revealed by single-molecule FRET analysis. Nature structural & molecular biology 2018, 25 (4), 347-354.
121. Babu, K.; Kathiresan, V.; Kumari, P.; Newsom, S.; Parameshwaran, H. P.; Chen, X.; Liu, J.; Qin, P. Z.; Rajan, R., Coordinated Actions of Cas9 HNH and RuvC Nuclease Domains Are Regulated by the Bridge Helix and the Target DNA Sequence. Biochemistry 2021, 60 (49), 3783-3800.
122. Bravo, J. P.; Liu, M.-S.; Hibshman, G. N.; Dangerfield, T. L.; Jung, K.; McCool, R. S.; Johnson, K. A.; Taylor, D. W., Structural basis for mismatch surveillance by CRISPR-Cas9. Nature 2022, 603 (7900), 343-347.
123. Raper, A. T.; Stephenson, A. A.; Suo, Z., Functional insights revealed by the kinetic mechanism of CRISPR/Cas9. Journal of the American Chemical Society 2018, 140 (8), 2971-2984.
124. Maher, R. L.; Bloom, L. B., Pre-steady-state kinetic characterization of the AP endonuclease activity of human AP endonuclease 1. Journal of Biological Chemistry 2007, 282 (42), 30577-30585.
125. Schermerhorn, K. M.; Delaney, S., Transient-state kinetics of apurinic/apyrimidinic (AP) endonuclease 1 acting on an authentic AP site and commonly used substrate analogs: the effect of diverse metal ions and base mismatches. Biochemistry 2013, 52 (43), 7669-7677.
126. Aboelnga, M. M.; Wetmore, S. D. J. J. o. t. A. C. S., Unveiling a single-metal-mediated phosphodiester bond cleavage mechanism for nucleic acids: a multiscale computational investigation of a human DNA repair enzyme. 2019, 141 (21), 8646-8656.
127. Hu, Q.; Jayasinghe-Arachchige, V. M.; Zuchniarz, J.; Prabhakar, R. J. F. i. c., Effects of the metal ion on the mechanism of phosphodiester hydrolysis catalyzed by metal-cyclen complexes. 2019, 7, 195.
128. Kamerlin, S. C.; Sharma, P. K.; Prasad, R. B.; Warshel, A. J. Q. r. o. b., Why nature really chose phosphate. 2013, 46 (1), 1-132.
129. O'Ferrall, R. M. J. J. o. t. C. S. B. P. O., Relationships between E 2 and E 1c B mechanisms of B-elimination. 1970, 274-277.
130. Lassila, J. K.; Zalatan, J. G.; Herschlag, D. J. A. r. o. b., Biological phosphoryl-transfer reactions: understanding mechanism and catalysis. 2011, 80, 669-702.
131. Babu, K.; Kathiresan, V.; Kumari, P.; Newsom, S.; Parameshwaran, H. P.; Chen, X.; Liu, J.; Qin, P. Z.; Rajan, R. J. B., Coordinated Actions of Cas9 HNH and RuvC Nuclease Domains Are Regulated by the Bridge Helix and the Target DNA Sequence. 2021, 60 (49), 3783-3800.
132. Senn, H. M.; O'Hagan, D.; Thiel, W. J. J. o. t. A. C. S., Insight into enzymatic C—F Bond formation from QM and QM/MM calculations. 2005, 127 (39), 13643-13655.
133. Slaymaker, I. M.; Gao, L.; Zetsche, B.; Scott, D. A.; Yan, W. X.; Zhang, F. J. S., Rationally engineered Cas9 nucleases with improved specificity. 2016, 351 (6268), 84-88.
134. Bravo, J. P.; Liu, M.-S.; Hibshman, G. N.; Dangerfield, T. L.; Jung, K.; McCool, R. S.; Johnson, K. A.; Taylor, D. W. J. N., Structural basis for mismatch surveillance by CRISPR-Cas9. 2022, 603 (7900), 343-347.
135. Wang, J.; Skeens, E.; Arantes, P. R.; Maschietto, F.; Allen, B.; Kyro, G. W.; Lisi, G. P.; Palermo, G.; Batista, V. S. J. B., Structural Basis for Reduced Dynamics of Three Engineered HNH Endonuclease Lys-to-Ala Mutants for the Clustered Regularly Interspaced Short Palindromic Repeat (CRISPR)-Associated 9 (CRISPR/Cas9) Enzyme. 2022.
136. Tsai, S. Q.; Nguyen, N.; Zheng, Z.; Joung, J. K. J. N., High-fidelity CRISPR-Cas9 variants with undetectable genome-wide off-targets. 2016, 529 (7587), 490-495.
137. Zuo, Z.; Babu, K.; Ganguly, C.; Zolekar, A.; Newsom, S.; Rajan, R.; Wang, Y.-C.; Liu, J. J. T. C. J., Rational Engineering of CRISPR-Cas9 Nuclease to Attenuate Position-Dependent Off-Target Effects. 2022, 5 (2), 329-340.
138. Vakulskas, C. A.; Dever, D. P.; Rettig, G. R.; Turk, R.; Jacobi, A. M.; Collingwood, M. A.; Bode, N. M.; McNeill, M. S.; Yan, S.; Camarena, J. J. N. m., A high-fidelity Cas9 mutant delivered as a ribonucleoprotein complex enables efficient gene editing in human hematopoietic stem and progenitor cells. 2018, 24 (8), 1216-1224.
139. Casini, A.; Olivieri, M.; Petris, G.; Montagna, C.; Reginato, G.; Maule, G.; Lorenzin, F.; Prandi, D.; Romanel, A.; Demichelis, F. J. N. b., A highly specific SpCas9 variant is identified by in vivo screening in yeast. 2018, 36 (3), 265-271.
140. Lee, J. K.; Jeong, E.; Lee, J.; Jung, M.; Shin, E.; Kim, Y.-h.; Lee, K.; Jung, I.; Kim, D.; Kim, S. J. N. c., Directed evolution of CRISPR-Cas9 to increase its specificity. 2018, 9 (1), 1-10.
141. Bratovič, M.; Fonfara, I.; Chylinski, K.; Gálvez, E. J.; Sullivan, T. J.; Boerno, S.; Timmermann, B.; Boettcher, M.; Charpentier, E. J. N. C. B., Bridge helix arginines play a critical role in Cas9 sensitivity to mismatches. 2020, 16 (5), 587-595.
142. Babu, K.; Amrani, N.; Jiang, W.; Yogesha, S.; Nguyen, R.; Qin, P. Z.; Rajan, R. J. B., Bridge helix of Cas9 modulates target DNA cleavage and mismatch tolerance. 2019, 58 (14), 1905-1917.
143. Chen, J. S.; Dagdas, Y. S.; Kleinstiver, B. P.; Welch, M. M.; Sousa, A. A.; Harrington, L. B.; Sternberg, S. H.; Joung, J. K.; Yildiz, A.; Doudna, J. A. J. N., Enhanced proofreading governs CRISPR-Cas9 targeting accuracy. 2017, 550 (7676), 407-410.

1. A modified Cas9 protein, wherein the modified Cas9 protein comprises at least one modification, the modifications include one or more of Lys896 and/or Lys253 corresponding to the amino acid of SEQ ID NO:1.

2. The modified Cas9 protein of claim 1, further comprising one or more modification that includes modification of Arg820, Arg400, Lys855 Thr58, Glu60, Glu223, Glu370, Glu371, Asp406, Glu396, Glu584, Asp585, Arg586, Arg765, Asn767, Arg778, Glu779, Gln807, Tyr812, Gln844, Ser845, Arg859, Lys263, Lys902, Arg864, Lys866, Lys918, Asn14, Lys268, Arg447, Tyr450, Asn497, Lys500, Lys526, Lys528, Lys558, Asn588, Arg661, Asn692, Gln695, Arg780, Arg783, Asn803, Gln805, Lys810, Asp829, Asn831, Arg832, Asp835, Lys848, Lys862, Arg925, Gln926, Lys929, His930, Lys961, Lys968, Tyr1013, Lys1031, Lys1244, or Lys 1246 corresponding to SEQ ID NO:1.

3. The modified Cas9 protein of claim 1, wherein the modified Cas9 protein contains at least two amino acid modifications.

4. The modified Cas9 protein of claim 2, wherein the modified Cas9 protein contains at least three amino acid modifications.

5. The modified Cas9 protein of claim 2, wherein the modified Cas9 protein contains at least four amino acid modifications.

6. The modified Cas9 protein of claim 1, wherein the Cas9 modification comprises K896 and K253.

7. The modified Cas9 protein of claim 1, wherein the Cas9 modification comprises K896.

8. The modified Cas9 protein of claim 1, wherein the Cas9 modification comprises K253.

9. The modified Cas9 protein of claim 1, wherein the modification is a substitution with an alanine, glycine, arginine, aspartic acid, or glutamic acid.

10. The modified Cas9 protein of claim 1, further comprising a nuclear localization signal, a cell penetrating amino acid sequence, or an affinity tag.

11. The modified Cas9 protein of claim 1, wherein the wild-type Cas9 protein is a Streptococcus pyogenes Cas9 protein.

12. A fusion protein comprising the modified Cas9 protein of claim 1 fused to a heterologous peptide or protein, with an optional intervening linker.

13. An expression cassette encoding the modified Cas9 protein of claim 1.

14.-16. (canceled)

17. A method of altering the genome of a cell, the method comprising expressing in the cell or contacting the cell with the modified Cas9 protein of claim 1, linked to a guide RNA having a region complementary to a selected portion of the genome of the cell, resulting in the alteration of the genome of the cell.

18. A method of altering a double stranded DNA (dsDNA) molecule, the method comprising contacting the dsDNA molecule with the modified Cas9 protein of claim 1, linked to a guide RNA having a region complementary to a selected portion of the dsDNA molecule, resulting in the alteration of the dsDNA molecule.

19. A system comprising a Cas9 protein, a guide RNA, and a target DNA strand comprising a C to G mismatch at a fifth position of a protospacer adjacent motif (PAM) as compared to the guide DNA strand.

20. The system of claim 19, wherein the Cas9 protein comprises at least one modification, the modifications include one or more of Lys896 and/or Lys253 corresponding to the amino acid of SEQ ID NO: 1.

21. The system of claim 19, wherein the target DNA strand is comprised in a double stranded DNA that further comprises a non-target DNA strand.

22. The system of claim 21, wherein the non-target DNA strand comprises a C at a nucleotide corresponding to the fifth position of the protospacer adjacent motif (PAM) of the target DNA strand.

23. A method of modeling a Cas9 HNH domain comprising modeling the system of claim 19.
